(19) United States
(12) Patent Application Publication
(10) Pub. No.: US 2025/0364145 A1
(43) Pub. Date: Nov. 27, 2025

(54) MONITORING QUALITY OF PHARMACEUTICAL MANUFACTURING SITES

(71) Applicant: PIRAMAL PHARMA LIMITED, Mumbai (IN)

(72) Inventors: Rashida NAJMI, Bethlehem, PA (US); Kiran Shankar GAIKWAD, Mumbai (IN); Rohit SHARMA, Mumbai (IN); Sonu SINGH, Mumbai (IN)

(73) Assignee: PIRAMAL PHARMA LIMITED, Mumbai (IN)

(21) Appl. No.: 19/293,161

(22) Filed: Aug. 7, 2025

Related U.S. Application Data
(63) Continuation-in-part of application No. 16/697,387, filed on Nov. 27, 2019, now abandoned.
(60) Provisional application No. 62/790,500, filed on Jan. 10, 2019.

(30) Foreign Application Priority Data
Nov. 28, 2018 (IN) 201821044823

(51) Int. Cl. G16H 50/30; G16H 40/20; G16H 50/20; G16H 70/20

(52) U.S. Cl. CPC G16H 50/30; G16H 40/20; G16H 50/20; G16H 70/20

(57) ABSTRACT

The present invention relates to a method for quantitatively evaluating the quality health of pharmaceutical manufacturing sites; specifically, the invention pertains to a method that measures audit readiness, data integrity compliance, and overall operational performance of the pharmaceutical manufacturing sites.

### CROSS-REFERENCE TO RELATED APPLICATIONS

The present application is a continuation-in-part of U.S. Ser. No. 16/697,387, filed on Nov. 27, 2019, which claims the benefit of priority to U.S. Provisional Application No. 62/790,500, filed on Jan. 10, 2019, and Indian Provisional Application No.: 201821044823 filed on Nov. 28, 2018, the entire disclosures of each of which are incorporated herein by reference.

### FIELD OF THE INVENTION

The present invention relates to a method for quantitatively evaluating the quality health of pharmaceutical manufacturing sites; specifically, the invention pertains to a method that measures audit readiness, data integrity compliance, and overall operational performance of the pharmaceutical manufacturing sites.

### BACKGROUND OF THE INVENTION

Pharmaceutical products, by their very nature, require a high level of quality assurance, given their direct impact on patient safety and therapeutic outcomes. As prophylactic or curative agents, such products must not only meet stringent efficacy and safety standards but must also be manufactured under rigorously controlled conditions.

Unlike other high-risk industries such as automotive or aerospace, where failures can result in immediate and visible consequences, lapses in pharmaceutical quality may remain undetected for years. This latent risk underscores the necessity of a robust and proactive quality system that can sense, quantify, and address compliance weaknesses before they manifest into product or regulatory failures.

Globally, pharmaceutical regulatory requirements are still undergoing harmonization. Agencies such as the USFDA (United States Food and Drug Administration), EMA (European Medicines Agency), MHRA (Medicines and Healthcare products Regulatory Agency), and WHO (World Health Organization), each prescribe detailed guidance, but often with region-specific expectations and terminologies. For globally operating pharmaceutical companies, aligning internal quality systems to meet diverse regulatory standards is complex and resource intensive. Traditional methods for monitoring compliance such as internal audits, metric dashboards, and manual reviews of Standard Operating Procedure (SOP) are often isolated, subjective, and retrospective. These methods rely heavily on sampling and auditor interpretation, introducing variability and limiting their predictive value. Moreover, tracking quality metrics in isolation does not provide a comprehensive view of a site's health or its readiness for regulatory inspection.

There exists a critical need for a structured, repeatable, and quantifiable approach that integrates key compliance indicators into a single, tangible outcome, a “site health score”, that reflects both current performance and regulatory risk. The ability to interpret such a score scientifically and act upon it can enable targeted remediation, efficient resource allocation, and improved audit preparedness.

Recognizing this gap, the inventors have developed the present method to improve consistency, transparency, and regulatory preparedness across its global pharmaceutical manufacturing network.

### SUMMARY OF THE INVENTION

In one aspect, the present invention provides a structured, scientific method for evaluating compliance and quality health of pharmaceutical manufacturing sites.

In another aspect, the present invention relates to a method comprising an Excel-based analytical framework designed to calculate site-level scores that reflect audit readiness, data integrity compliance, and overall operational performance, wherein the method integrates tangible compliance metrics into a unified scoring model to enable predictive assessment of a pharmaceutical manufacturing site's regulatory preparedness and quality risks.

In yet another aspect, the present invention provides a method of measuring the quality health of a pharmaceutical manufacturing site, wherein the quality is visually measured on a level meter, referred to as SENSOR™ and is determined as a dynamic site score on a scale from 0 to 5, which is determined by consolidating eleven critical quality indicators, each assigned a predefined rationalized weightage, wherein said score. is interpreted to classify sites by their health status, required level of remediation and associated compliance risk.

In a further aspect, the present invention relates to a method of evaluating the ‘Risks’ involved and ‘Focus’ required for a pharmaceutical manufacturing site under evaluation as described herein.

In another aspect, the present invention provides a method of evaluating the audit readiness of a pharmaceutical manufacturing site, wherein the outcome of the method is determined in the form of a rating obtained via a graphical representation, referred to as PREDICT™ rating which method comprises evaluating of three core elements:
1. A System Score calculated using 132 parameters across six Good Manufacturing Practice (GMP) systems in alignment with regulatory inspection models such as USFDA's Six-System approach.
2. A Subject Matter Expert (SME) Readiness Score representing the site's subject matter expert preparedness during audits.
3. A Data Integrity Compliance Score derived from CALCULUS™.

The PREDICT™ rating provides insight into probable audit outcomes and helps in aligning site focus with the expectation of the audit outcome.

In a further aspect, the present invention provides a method of evaluating data integrity compliance of a pharmaceutical manufacturing site, wherein the outcome of the method is determined in the form of score in percentage referred to as CALCULUS™ corresponding to a rating, which evaluates adherence to global regulatory guidelines such as those issued by USFDA, MHRA, WHO, and Pharmaceutical Inspection Co-operation Scheme (PIC/S). The CALCULUS™ score is derived from two components:
A checklist-based compliance assessment referred as the Data Integrity (DI) Score (85% weightage)
The extent of automation across Quality Control (QC), Quality Management System (QMS), and Manufacturing (Mfg) systems, referred as the Automation score (15% weightage).

The pharmaceutical manufacturing sites are rated based on percentage compliance and classified according to predefined DI compliance thresholds.

The present invention thus offers a comprehensive quality compliance intelligence method that not only captures pharmaceutical manufacturing site status at a point in time but also helps predict regulatory vulnerabilities and prioritize quality remediation efforts. The invention can be used across multiple pharmaceutical manufacturing sites to harmonize quality governance, reduce audit surprises, and support strategic quality oversight within organizations. An illustrative diagram depicting the overall methodology/approach is given in FIG. 1.

These and other aspects of the invention will be more fully understood from the following detailed description, figures, and examples.

### BRIEF DESCRIPTION OF DRAWINGS OF THE INVENTION

FIG. 1 represents an illustrative diagram depicting the structured method for evaluating compliance and quality health of a pharmaceutical manufacturing site.

FIG. 2A represents the graphical plotting of the initial PREDICT™ Score for 12 pharmaceutical manufacturing sites

FIG. 2B represents the graphical plotting of the improved PREDICT™ Score for 12 pharmaceutical manufacturing sites

FIG. 3 represents comparative evaluation of SENSOR™ Score for 12 pharmaceutical manufacturing sites.

FIG. 4A represents initial SENSOR™ Score for pharmaceutical manufacturing site #9.

FIG. 4B represents improved SENSOR™ Score for pharmaceutical manufacturing site #9.

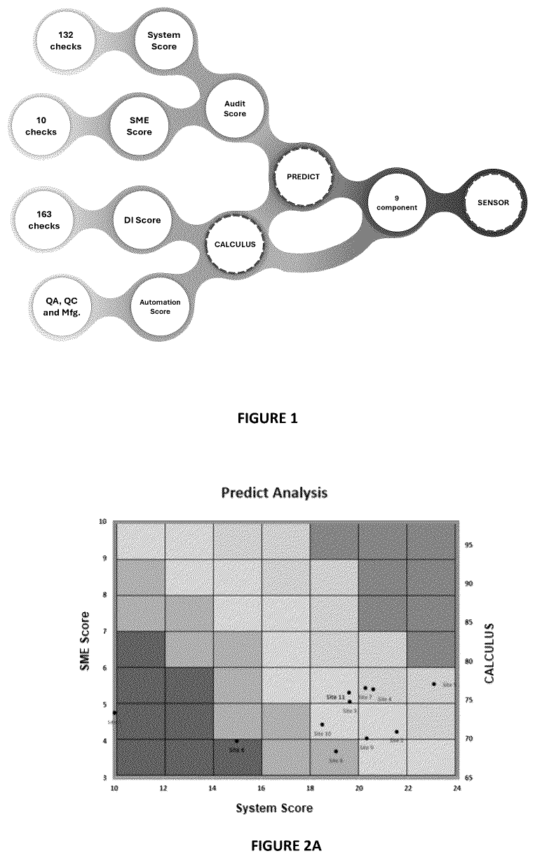

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the description herein, may utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. The present invention provides a structured methodology and scoring framework for evaluating the quality health of pharmaceutical manufacturing sites using quantifiable data. The method of the instant invention is implemented via a spreadsheet-based tool (e.g., Microsoft Excel) programmed with complex formulas, predefined thresholds, and weighted scoring logic.

### Definitions

For the purposes of this invention, the following terms are defined as follows. These definitions apply throughout the specification unless otherwise indicated.

It should be noted that, as used in this specification and the appended claims, the singular forms “a,” “an,” and “the” include plural referents unless the content clearly dictates otherwise.

It should be noted that the term “or” is generally employed in its sense including “and/or” unless the content clearly dictates otherwise.
CALCULUS™: A proprietary data integrity compliance assessment module that computes a percentage compliance score for a site based on regulatory checklist compliance (DI score) and level of automation (Automation score). Throughout the specification, the words, ‘parameter(s)’ and ‘checkpoint(s)’ are used interchangeably.
PREDICT™: A predictive audit readiness assessment model composed of three elements: System Score (based on six GMP systems), SME Readiness Score, and CALCULUS™ Score. The combined outcome is used to simulate regulatory audit preparedness. Throughout the specification, the words, ‘parameter(s)’, ‘subsystem(s)’ and ‘checkpoint(s)’ are used interchangeably.
SENSOR™: A level meter that integrates eleven weighted compliance indicators including the PREDICT™ score to generate a final site score on a scale from 0 to 5, representing the overall quality health status. Throughout the specification, the words, ‘parameter(s)’, ‘indicator(s)’ and ‘critical indicator(s)’ are used interchangeably.
Data Integrity (DI): The extent to which all Good Practices (GxP) related data is accurate, complete, consistent, and secure throughout the data lifecycle. Assessed per global regulatory guidance including USFDA, WHO, MHRA, and PIC/S.
Subject Matter Expert (SME): A designated personnel evaluated on their audit-facing preparedness and technical ability to represent site systems during inspections.
System Score: A numeric rating calculated by evaluating site performance across 132 parameters spanning six GMP systems, each weighted by its risk to patient safety, product quality, or compliance.
Checklist Score or Data Integrity (DI) Score: The component of the CALCULUS™ model evaluating regulatory alignment across 163 defined data integrity parameters.
Automation Score: The component of the CALCULUS™ model reflecting the degree of digital automation in Quality Control, Quality Assurance, and Manufacturing operations.

The present invention relates to a method which is modular in design and includes three core components: CALCULUS™, PREDICT™, and SENSOR™, each serving a distinct function in the quality assessment process.

In an aspect, the present invention relates to a method for evaluating the data integrity compliance, audit readiness and/or quality health of a pharmaceutical manufacturing site by determining the CALCULUS™ score, PREDICT™ rating and SENSOR™ score respectively.

In one aspect, the present invention relates to a method of evaluating data integrity compliance of a pharmaceutical manufacturing site, wherein the outcome of the method is determined in the form of a score in percentage referred to as CALCULUS™ corresponding to a rating, which evaluates adherence to global regulatory guidelines such as those issued by USFDA, MHRA, WHO, and PIC/S. The CALCULUS™ score is derived from two components:
A checklist-based compliance assessment referred as the Data Integrity (DI) Score (85% weightage)
The extent of automation across Quality Control, Quality Assurance, and Manufacturing systems, referred as the Automation score (15% weightage).

The component CALCULUS™ of the present invention is explained herein in detail:

### Significance of Data Integrity Assessment and Compliance:

In pharmaceutical industry, ensuring data integrity compliance with regulatory standards is paramount for patient safety and drug efficacy. Requirements for Data Integrity compliance are released by the USFDA, MHRA, WHO and other regulatory agencies. Even though regulatory requirements are easily available, many pharmaceutical companies have received import ban due to Data Integrity lapses at their facilities and this has resulted in drug shortages, thereby impacting end patients (no medicine or costly medicines). The pharmaceutical companies which failed to comply with the Data Integrity regulations, were not able to keep pace with the changes in regulations and were verifying the requirements without a structured method. To avoid any impact to end patients, the inventors of the present invention formulated a method to check the Data Integrity compliance of pharmaceutical manufacturing sites and evaluate the sites through a methodical process without human bias. The innovative method designed evaluates data integrity compliance through system checkpoints/parameters and automation levels at the pharmaceutical manufacturing sites. The method further provides a systematic approach to collate data and verify its accuracy, consistency, and reliability across all stages of drug development and manufacturing.

The method of evaluation of data integrity compliance or CALCULUS™ Score comprises the steps of:

a. Development of Checkpoints:
Different guidelines on Data Integrity are published by various regulatory bodies namely WHO, USFDA, MHRA, EMA, PIC/S, ISPE (International Society for Pharmaceutical Engineering). To evaluate the compliance level against each regulatory requirement is a difficult task leading to lapses during execution. Based on the regulatory guidelines, the inventors of the present invention have drafted the checkpoints (total 163 nos.) of the instant invention. These checkpoints were strategically drafted and detailed, without being directly verbatim present in any of the above regulatory guidelines.
In addition to current regulatory requirements, Automation level in QC (High-Performance Liquid Chromatography (HPLC), Laboratory Information Management System (LIMS), Server based system), Manufacturing (Supervisory Control And Data Acquisition (SCADA), Programmable Logic Controller (PLC), Manufacturing Execution System (MES)) and QA (Electronic Document Management System (eDMS), Learning Management System (LMS), Aberration Handling system) were also included in the methodical evaluation to ensure adoption of latest technology, compliance with future requirements and move from manual controls to design controls (automation/digitization).

### B. Categorization of Checkpoints:

To facilitate a structured approach for targeted improvements and efficient compliance management, the 163 parameters used to evaluate the data integrity compliance of a pharmaceutical manufacturing site are selected from the categories as under:
a) Recording and collection of data
b) Original Record and True Copy
c) Excluding Data
d) Data Processing
e) Data Transfer/migration
f) Data Governance
g) Data Integrity Risk Assessment (DIRA)
h) Computerised system transactions
i) Computerised system User access/System administrator roles
j) Audit trail
k) Electronic signatures
l) Data review and approval
m) Data Retention
n) Backup & Archive
o) File structure
p) Validation
q) IT Suppliers and Service Providers
r) Quality Management System (QMS)
s) Calibration
t) Quality Control
u) Standalone systems
v) Trainings
w) Manufacturing process

The above categories are further sub-categorized into 163 parameters.
Out of the 163 parameters, 14 parameters (40%) are categorized as ‘Critical’ and are mandatory to be compliant. If any parameter belonging to this category at any given point of time is not compliant, then the score for this complete mandate section becomes zero even though other parameters within mandate section are compliant. Hence, the score falls by 40%.
All the 14 critical parameters are assigned weightage of 2.86% each, leading to total of 40% score and the rest 149 parameters are categorized as essential, of which each parameter is given 0.4% rating.
The 149 parameters are also essential, however at any given point of time if these 149 parameters are non-compliant/or partially compliant, they can be accepted with adequate justification, risk assessment and mitigation plan.

The data integrity (DI) score is determined using data integrity compliance checklist of 163 parameters comprising:

### (a) Recording and Collection of Data:

1. Checking whether an Standard Operating Procedure (SOP) or guideline on Good Documentation Practices exists, whether it covers aspects of ‘ALCOA’ (Attributable, Legible, contemporaneous, Original, Accurate) & ‘+’ (Complete, Consistent, Enduring, Available) principle and whether they are followed;
2. Checking whether an SOP for Issuance, document control and reconciliation & destruction of Blank forms/Batch records, paginated logbooks and Lab Notebooks exists;
3. Checking whether a process of ensuring that always the current copy of SOP is in circulation and the obsolete SOPs are withdrawn in timely manner with appropriate reconciliation is in place;
4. Checking whether uncontrolled copies are issued and if issued, whether the SOP defines the procedure on issuance and handling of uncontrolled copies;
5. Checking whether issuance of Batch Manufacturing Record (BMR), Batch Packaging Record (BPR) and lab notebooks is controlled by Quality Assurance (QA), Document controller or by electronic module and whether each page is either stamped and signed or bears a unique identification with page number (auto generated);
6. Checking whether there is a proper control over replacement of any page(s) and in times of genuine need, whether this is done through a validated system control and whether replacement is recorded adequately;
7. Checking whether there is a logbook maintained for issuance of forms, templates or similar documents;
8. Checking whether all Quality related activities are recorded at the same time when they are performed, and whether BMR, BPR and Lab notebooks are recorded contemporaneously;
9. Checking whether the site has a practice of recording data on pieces of paper that will be discarded after the data are transcribed to a permanent laboratory notebook;
10. Checking whether there is a practice of use of scribes to record activity on behalf of another operator, if such practice exists, whether it is verified and documented contemporaneously or retrospectively with adequate justification, and whether this is defined as part of procedure;
11. Checking whether there exist practices to ensure in routine and confirm that there is no backdating, postdating, omitting negative data (Out of Specification (OOS) or eliminating outliers) or releasing failing product, manipulation, deliberated hidden information or missing signatures on any Good Manufacturing Practices (GMP) or Quality Management System (QMS) record;
12. Checking whether the team has appropriate level of process understanding and technical knowledge of systems used for data collection and recording, including their capabilities, limitations and vulnerabilities;
13. Checking whether the system has feature to not obscure previously recorded information during record change by authorized personnel;
14. Checking whether adequate controls exist in case of run abortion in High Performance Liquid Chromatography (HPLC) systems;
15. Checking whether electronic system (HPLC, QMS application, server) downtime are documented and investigated for the reason;
16. Checking whether key process equipment have controls that measure its operating range of critical process parameters and whether this range is reviewed during batch release decision;
17. Checking whether hybrid systems are used and if used, whether they are clearly documented for which data set is to be reviewed and retained;
18. Checking whether the system has the ability to generate accurate and complete data records in both paper and electronic format (human readable) which could be readily available and accessible (if requested by national competent authorities);
19. Checking whether the system generated report specifies the time zone where this data is used across multiple sites;
20. Checking whether there exists a practice of retention of incomplete or erroneous forms as permanent records along with justification of their replacement and whether this is governed by SOP;
21. Checking whether records or procedures are accessible to locations where activities take place;

### (b) Original Record or True Copy:

1. Checking whether there exists a system or procedure of Handling, Review and Retention of original records-dynamic (electronic) records and static (printed/manual) records generated at Site;
2. Checking whether the data (or a true copy) generated in paper format is retained in the form of scan copies;
3. Checking whether there exists any data at the site where it is not feasible to retain in original form, and if it such data exists, whether the risk assessment is documented and whether appropriate form of data is retained as true copy;
4. Checking whether a true copy of the original data is maintained securely throughout the records retention period, whether there is a risk assessment performed in case of destruction of original record and whether this is governed by SOP;
5. Checking whether there exists a process of risk assessment of data where the data obtained requires manual observation to record the results of manual titration, visual interpretation of environmental monitoring plates and similar results;
6. Checking whether manual transcription for above point are verified by second person or validated system;
7. Checking whether the true copy is stored in different electronic format to the original record and if stored in a different format, whether retention of meta data and audit trail are ensured;

### (c) Excluding Data:

1. Checking whether there is a process for exclusion of data and if such process exists, whether there is a process to demonstrate through valid scientific justification that the data are not representative of the quantity measured, sampled or acquired;

### (d) Data Processing:

1. Checking whether reprocessing of chromatographic data is performed and if reprocessed, whether there exist procedures in place to handle and document such reprocessing;
2. Checking whether a copy is saved only of the final results from reprocessed laboratory chromatography and not the entire sequence;
3. Checking whether manual integration of chromatograms is practiced and whether the software allows saving of manually integrated chromatograms or test results on desktops or temporary locations;
4. Checking whether there exist controls to track or check if trial samples are analysed before the original sample;

### (e) Data Transfer or Migration:

1. Checking whether the site team has a robust and validated data transfer or migration or retention procedure to ensure that data integrity is maintained during the data lifecycle;
2. Checking whether there is a data migration audit log, whether there were challenge tests performed on the migrated data to ensure there is no alteration at each stage of data generation;
3. Checking whether the migration failures (if any) are logged through QMS;

### (f) Data Governance:

1. Checking whether there exists an SOP on Data Integrity which defines data governance at the site and whether the data ownership and accountability is defined in the SOP;
2. Checking whether the procedure describes the method to handle data integrity incident observed (if any) at the site;
3. Checking whether periodic audits are conducted for data integrity failures within the organisation's systems;
4. Checking whether the DI behaviour aspects are a part of performance indicators;
5. Checking whether the site performs a data governance review as part of their vendor assurance programme;

### (g) Data Integrity Risk Assessment (DIRA):

1. Checking whether DIRA is performed at the site;
2. Checking whether data integrity risk assessment (or equivalent) considers factors required to follow a process or perform a function including not only a computerised system but also the supporting people, guidance, training and quality systems;
3. Checking whether there are procedures in place to minimize the potential risk to data integrity including identifying the residual risk using risk management techniques;
4. Checking whether the site has a core team or SME who performs the DIRA;
5. Checking whether the DIRA report highlights areas for remediation including,
   1) documentation of prioritisation of actions (including acceptance of an appropriate level of residual risk),
   2) communication to management, subject to review, and
   3) implementing risk-reducing short-term measures in situations where long-term remediation actions are identified to provide acceptable data governance in the interim;

### (h) Computerized System Transactions:

1. Checking whether electronic modules (document management or Trackwise) used for QMS system are 21 CFR part 11/EU annex 11 compliant;
2. Checking whether the systems used at site are capable of saving data to permanent memory before prompting users to make changes;
3. Checking whether in case of transactional systems, any combination of multiple unit operations into a combined single transaction is avoided and whether the time interval before saving the data in computerized system is minimized;
4. Checking whether there exists a process of changing time due to day light savings and if it exists, whether there is a procedure and controls in place, whether this is done by authorized individual and whether this is documented;
5. Checking whether the date and time change setting is disabled at all computer workstations on site;
6. Checking whether time management system is validated to ensure correct logging of date and time of attendance;
7. Checking whether there exists a procedure detailing the password complexity, number of wrong password attempts and password change frequency (system generated);
8. Checking whether password change prompt is activated asking for the change on fixed periodicity;
9. Checking whether the computer system gets timed out within limited time if not worked on as per Information Technology (IT) policy requiring re-entering of password, and if the user leaves the workstation, whether procedures and/or automatic controls define and ensure that it is treated as a non-continuous session;

### (i) Computerized System User Access/System Administrator Role:

1. Checking whether there exists a procedure to create and maintain user account in electronic system and whether the system has control to avoid duplicate user creation;
2. Checking whether there exists an approved user access matrix for all the users and whether this access matrix exactly corresponds with the actual user privileges while using the systems;
3. Checking whether the access controls are applied to both the operating system and application levels and whether the access privileges list are periodically updated;
4. Checking whether sufficient controls are available to prevent unauthorized access to system or to change or delete the data;
5. Checking whether there exist instruments or computer systems that have shared logins, whether generic user or system admin access is used for generating, amending or storing data for Good Practices (GxP) systems and whether there are appropriate procedures available to ensure user controls and data integrity;
6. Checking whether all users have individual login ID and password to the respective instrument or system where electronic data or record is generated or maintained and in absence thereof, whether appropriate controls are in place to ensure data integrity;
7. Checking whether the system administrator for all computerised systems is independent from the user and whether same person acting as user as well as administrator;
8. Checking whether there are multiple system administrators for a single system and if there are such multiple system administrators, whether they are restricted to minimum number of people and whether they use individual ID and password;
9. Checking whether only system administrator has rights to alter files and settings and if they have such rights, whether there exists record of all the actions generated;
10. Checking whether appropriate assessments and controls are available for systems partially used for GxP purposes, approved suppliers, stock status, location and transaction histories and similar purposes;
11. Checking whether if no suitable alternative computerised system is available, whether equivalent controls are provided by third-party software or a paper-based method of providing traceability (with version control);

### (j) Audit Trail:

1. Checking whether audit trail is activated for all your computerised systems;
2. Checking whether Audit trail includes the following:
   User Name
   Date and Time
   Reason for change
   Integration parameter used in case of chromatograph
   Reprocessing details
   Change History
   Changes to sample run sequence (as applicable)
   Change in critical process parameters to achieve a more desirable result (as applicable)
   esignature;
3. Checking whether user is able to amend (switch on/off) the audit trail settings and edit the raw data (including chromatographic data) for any computerized systems;
4. Checking whether there exist optimal controls and measures to evaluate data generated by the message centre in the form of an audit trail related to alarms, warning, error or run time data and whether there is an SOP governing the same;
5. Checking whether the electronic record has system generated date and time stamp, and if the time on the system and report is synchronized;
6. Checking whether there exists a procedure to periodically review the appropriate audit trails and data integrity review to verify adherence to written procedures (to verify privileges assigned to users, creation of project folders, data generated and maintained correctly);
7. Checking whether there exists a procedure in place that describes audit trail review requirements:
   frequency and documentation of its review,
   date of review, reviewed by and outcome of review, identification of actions required in case of non compliance,
   indicators of unauthorized changes;
8. Checking whether there are enough controls to ensure that all original chromatographic data is restricted from editing once finalized or approved or locked;
9. Checking whether the editing of point (j)8 is detectable in audit trails and whether such aspects are considered as part of review of audit trail;

### (k) Electronic Signatures:

1. Checking whether there exists an SOP or policy on use or handling of e-signatures;
2. Checking whether there exists an appropriate validation of signature process associated with the system;
3. Checking whether a record is maintained of all the associates who apply their e-signature instead of their handwritten signatures;
4. Checking whether all the computerised systems where electronic signatures are applied, document the specific person or title who signed the records electronically along with date and time stamp and the meaning of the signature (verified or approved);
5. Checking whether there exists a process of inserting image of the signature or a footnote indicating that the document has been electronically signed (where this has been entered by a means other than the validated electronic signature process);

### (l) Data Review and Approval:

1. Checking whether there exists a procedure that describes the process for review and approval of data and whether the raw data is accessible with appropriate control to person performing data checking activities on system;
2. Checking whether the data review includes risk-based review of relevant meta data including audit trails and whether the same is documented;
3. Checking whether there exists a practice of thorough data review for all applicable electronic source data to verify scientific integrity of the reported results, to verify adherence to procedures or test methods and to verify that all results are reported and accounted for; whether there is an SOP governing the same;
4. Checking whether the equipment raw data is periodically reviewed, reconciled against paper records and extracted as electronic data where the equipment does not store electronic data permanently and only holds a certain volume before overwriting;
5. Checking whether there is any procedure for periodic review or audit of electronic data generated, which verifies the effectiveness of existing control measures and the possibility of unauthorised activity;
6. Checking whether there are controls to verify the reported data and actual data;
7. Checking whether customized reports in computer systems are validated and locked to prevent changes;

### (m) Data Retention:

1. Checking whether there is a retention policy for of all records (manual & electronic), soft data and meta data, and whether it covers periodicity for back up and retention of both electronic and paper records along with meta data;
2. Checking whether there are procedures for destruction of data (blank forms, reports, training records, work sheets, raw data files, logbooks) which consider data criticality;
3. Checking whether there are data and document retention arrangements available to ensure the protection of records from deliberate or inadvertent alteration or loss;
4. Checking whether the data retention process of the sites include:
   verification and availability of copies of all raw data (lab notebook, logs, balance printouts), metadata,
   check for availability of relevant audit trail and result files,
   any variable software or system configuration settings specific to each record, and all data processing runs (including methods and audit trails) necessary for reconstruction of a given raw data set;
5. Checking whether metadata includes following components while retention:
   a date or time stamp for when the data were acquired,
   a user ID of the person who conducted the test or analysis that generated the data,
   instrument ID used to acquire the data,
   audit trails;
6. Checking whether BMRs and BPRs are centrally archived under QA custody. whether access to this is controlled and restricted (manually controlled or electronically controlled through combination lock, biometrics or access card);
7. Checking whether there is any data generation through photograph, image or any other media and if there is such data generation, whether there are procedures and controls to maintain their storage throughout the lifecycle;
8. Checking whether in case, where the original format cannot be retained due to degradation issues, whether there are alternative mechanisms for recording (photography or digitisation) and subsequent storage considered and selection rationale documented;
9. Checking whether the data (or a true copy) generated in paper format is retained by using a validated scanning process, and if so retained, whether there is a documented process in place to ensure that the outcome is a true copy;
10. Checking whether there are adequate controls to prevent data tamper or deletion and data loss within the software or outside the software application;
11. Checking whether the soft data can be located, retrieved, presented and interpreted in its original or acceptable form;

### (n) Back Up and Archive:

1. Checking whether validated procedures are available for data backup and archive, and if available, whether it includes,
   i. periodic integrity check,
   ii. accuracy of backup data,
   iii. readability of data or metadata and
   iv. the ability to restore the data as per its original validated state including legacy systems to confirm the continued support of legacy computerised systems;
2. Checking whether a procedure is in place for archival of records prior to decommissioning the computerised system;
3. Checking whether data security controls exist on manufacturing software or Programmable Logic Controllers (PLCs), and whether it is backed up periodically to prevent data loss in case of failure;
4. Checking where hybrid records are stored and whether references between physical and electronic records are maintained such that full verification of events is possible throughout the retention period;
5. Checking whether the archival area is suitably controlled and whether it is inspected at regular intervals;
6. Checking whether the site is using any third party service provider for archival of data and if used, whether there is a contract in place which defines the responsibilities for archiving and continued readability of the data throughout the retention period;
7. Checking whether the backup strategies for the data owners are documented;
8. Checking whether adequate back up control, disaster management and server redundancy for chromatographic data exists;
9. Checking whether periodic review or retrieval of chromatographic records is performed, if performed whether it is performed with limited access control and whether this is documented;
10. Checking whether disaster management exists for archival area where BMR or BPR are archived;

### (o) File Structure:

1. Checking whether there exists a process for file structure and naming conventions within the GxP environment for management of electronic data to facilitate effective data review and ensure that all results are reported and accounted for; including naming of folders or files, sequences and samples as applicable;
2. Checking whether there exists a written procedure to control data acquisition in appropriate project folders and whether there exist appropriate access controls on these folders;

### (p) Validation:

1. Checking whether computerized systems are validated as per regulatory requirements for their intended purpose;
2. Checking whether the extent of validation is based on justified and documented risk assessment;
3. Checking whether all PLCs containing recipes or work instructions are validated as per Good Automated Manufacturing Practice (GAMP) 5 or for 21 CFR part 11 controls;
4. Checking whether excel work sheets if used in Quality Control (QC) or any other function are validated and whether these cells are locked, access is controlled and saving or copying of these are disabled;
5. Checking whether validation for intended purpose ensures that the steps for generating the custom report accurately reflect those described in the data checking SOP;
6. Checking whether each workflow on all the computer systems or softwares in use are validated;
7. Checking whether the critical steps are defined based on the functionality of system at User Requirement Specification stage of a software;
8. Checking whether computer systems designed to ensure that the execution of critical steps are recorded contemporaneously;
9. Checking whether the functional verification demonstrates that the required information is consistently and completely presented;
10. Checking whether periodic review of validation status of systems is performed and documented;
11. Checking whether there exists a process of retirement of computerized systems governed by an SOP;

### (q) IT Supplier and Service Provider:

1. Checking whether there exists an inventory of all applicable computerised systems both live and retired and whether they are periodically reviewed;
2. Checking whether the site uses any ‘cloud’ or ‘virtual’ services from third party, and if used, whether there is an understanding of the services provided, ownership, retrieval, retention and security of the data;
3. Checking whether the contract or technical agreements of third party vendor include responsibilities of contract giver and acceptor, clause on data ownership, governance and accessibility;
4. Checking whether in case of cloud services, the geographical location laws are considered where the data is physically located;
5. Checking whether there exists a process of auditing the cloud and software service provider;
6. Checking whether there is an external vendor used for generating summary reports for the site or organization, and if such vendor exists whether the vendor is evaluated for the data integrity controls and processes prior to using the information;
7. Checking whether consultants are appointed at the site, and if appointed, whether there exist procedural controls to prevent access to company data through secured network;
8. Checking whether there exists business continuity for all the critical softwares used at site;
9. Checking whether the business continuity arrangement (manual or alternative system) is documented in contract and tested, and whether the audit of the service provider is performed;

### (r) Quality Management Systems (QMS):

1. Checking whether changes to qualified electronic system are routed through change management and risk assessment;
2. Checking whether all deviations or incidences immediately upon their discovery, are documented;
3. Checking whether the deviation investigations (not only failures and data errors) include determining and correcting the root cause of the deviation and a thorough impact assessment;
4. Checking whether the Information Technology related incident or deviations within QMS are logged and tracked;
5. Checking whether the OOS handling procedure mentions when retest is permitted and when approval to do so is required;
6. Checking whether there exists a practice of frequent invalidation of OOS, in case of invalidated OOS, whether a thorough investigation is undertaken for all, and whether there are repeated errors for invalidating the OOS;
7. Checking whether there exists an SOP for repeat testing which includes scientific justification and documentation requirements for the same;
8. Checking whether there exists a procedure in place to handle, if any abnormality or incident related to software is observed during operation;

### (s) Calibration:

1. Checking whether the simple electronic systems (pH meters, balances, thermometers and similar electronic systems) are calibrated;

### (t) Quality Control:

1. Checking whether the site has a practice of trial injection in chromatographic analysis and deletion of sequences;
2. Checking whether there exists a practice of using actual test samples for system suitability or whether any test or equilibration runs are carried out;

### (u) Standalone Systems:

1. Checking whether all QC chromatographic systems or standalone instruments (balances, moisture analyser, ultra violet (UV) spectrophotometer, pH meter) are 21 CFR Part 11/EU Annex 11 compliant, and whether there exist entry level controls in these computers followed by system level access controls;
2. Checking whether there exist any standalone instruments (balances, moisture meter, UV spectrophotometer, pH meter) which are not attached with printers, and if they exist, whether they are validated and with adequate controls for capturing, documenting and second level review of the data and audit trails;
3. Checking whether in case of non availability of audit trail functionality (standalone instruments) and individual user account expectations, whether there exists an alternate robust control procedure for data review and retention along with justification, including legacy systems;
4. Checking whether there exist plans to upgrade standalone systems, and whether the required budget is proposed;
5. Checking whether computers are connected to chromatographic system and/or Supervisory Control and Data Acquisition (SCADA) are disabled for download of any other application (excel, power point, word), whether it is used exclusively for the data acquisition system, and whether it is confirmed that it does not allow download or saving of any data and subsequent changes to it;
6. Checking whether there exists a back-up of standalone instruments and whether the mechanism is validated;

### (v) Trainings:

1. Checking whether all personnel accessing the system or involved with the system have adequate training and whether their training records are available, whether their responsibilities are defined and documented to carry out their assigned duties;
2. Checking whether Data Integrity training is performed as part of induction for new joinees;
3. Checking whether periodic trainings to all GxP relevant support functions on importance of Data Integrity principle is provided as part of a routine annual training program;
4. Checking whether the personnel who review and conduct data integrity checks are trained and qualified on data integrity requirements;
5. Checking whether the senior management team is trained on data integrity requirements;
6. Checking whether there exists an SOP, policies, trainings or awareness sessions at site that helps prevent sharing of passwords or IDs;

### (w) Manufacturing Process:

1. Checking whether there exist PLC controls or automation to all key manufacturing equipment, such that defective product can be rejected based on defined tolerances;
2. Checking whether the processes and their validation are robust enough and challenged adequately to not produce a non-compliant product;
3. Checking whether there exists a qualified SCADA (where available) system and whether SCADA print is attached in BMR to prevent manual recording;
4. Checking whether there exists an SOP to assess and document PLC alarms based on its criticality (nature, recording the duration and time) from an operational standpoint, and whether this is also reviewed by the quality unit;
5. Checking whether the PLC controls, alarms the user not only when the product is out of specification but when process steps out of the validated range;
6. Checking whether there exist digital clocks to record timing in the raw data or batch record, and if they exist, whether they are centrally controlled or synchronized for recording timed events specifying the time zone;
7. Checking whether for Active Pharmaceutical Ingredient (API) sites, whether critical parameters (temperature, pressure and pH) of reactors are automated, whether it prevents manual recording and whether print out from PLCs for these parameters are attached in BMR;
8. Checking whether equipment cleaning, sanitization and maintenance records are available and traceable at site;
9. Checking whether procedures are in place to monitor the output of the manufacturing processes that may be responsible for causing variability in the characteristics of in-process material and the drug product;
10. Checking whether the documents generated or printed from electronic systems in shopfloor are compliant to 21 CFR part 11.

### C. Weightage to Checkpoints:

To determine the CALCULUS™ score, weightage is assigned to check points as below:
163 Checkpoints contributing to 85%, and
Automation level in QC (5%), Manufacturing (5%) and QMS (5%)—Total contributing to 15%.

### D. CALCULUS™ Score:

CALCULUS™ score for a site determines the compliance level at site and helps identify areas for immediate attention/improvement as indicated in TABLE 1 below:

### Interpretation of CALCULUS™ Score:

Rating A+ is achieved by a site in following conditions:
   Minimum 84% in checklist and 15% in automation.
   85% in checklist and minimum 14% in automation.
Rating A is achieved by a site in following conditions:
   Minimum 70% in checklist and 15% in automation.
   85% in checklist and 0% in automation.
   Other combinations where the total is between 85 to 98.99%.
Any CALCULUS™ score <85% indicates the site is not meeting the qualifying criteria and needs immediate attention to improve compliance with Data Integrity requirements.
Rating A+ indicates site is highly compliant with regulatory requirements due to automation and design controls.
Rating A indicates site is compliant with regulatory requirements. However, there is scope of improvement with respect to procedural or design controls and automation.

### E. Rationale for 85:15% Weightage:

Assigning appropriate weightage to the checklist and automation requirements is crucial for balancing the fundamental compliance needs with the advantages of automation.
The 85-15% weightage on checkpoints along with the qualifying criteria for CALCULUS™ score provides an assurance on the Data Integrity compliance level at a site.
The 85%-15% weightage distribution strikes a balance between maintaining essential compliance (checklist) and leveraging technology (automation) for better data management and integrity.
The checklist (checkpoints) encompasses all regulatory requirements for data integrity compliance. However, it is essential to implement or adopt newer technologies and practices to strengthen the compliance level at their facility and always remain compliant.
Rationale for assigning 85% to the checkpoint: Failing to meet any of the checklist requirements poses significant risks, including data breaches, regulatory non-compliance, and potential harm to patients. Hence, these requirements carry substantial weight.
Rationale for assigning 15% to the automation level at the facility: Automation plays a critical role in reducing human errors (unintentional/intentional data integrity issue). Though automation in QC, manufacturing and QMS is not mandatory as per regulatory requirements, having automation provides higher assurance on data reliability, accuracy, and data integrity compliance. Automation also encourages sites to adopt latest technologies or design controls, promoting a forward-looking approach while ensuring that the core compliance requirements are not overshadowed.
Total automation level is attributed 15%, wherein QC, manufacturing and QMS are attributed with 5% each.
By increasing the total automation level above 15%, the compliance focus on current regulatory requirements would be reduced.
By reducing the total automation weightage below 5% for each vertical (QC, manufacturing, QMS), the efforts to embrace latest technology/design controls will be minimized and impact the site compliance in near future.

### F. Assessment of a Pharmaceutical Manufacturing Site:

The Data Integrity compliance checklist is annually filled by sites for each section against the current procedures, systems, practices, and automation level.
The filled checklist is then reviewed by corporate quality personnel who verify the compliance during the site audit.
Based on the final review and concurrence the scoring is done by combining the checklist score and automation score to provide a comprehensive and balanced assessment of data integrity compliance. This formula ensures that all aspects of compliance are considered, resulting in a holistic evaluation.

$${CALCULUS}^{TM} = ( 85 \% \times \frac{1}{163} {\sum }_{i = 1}^{163} {C}_{i} ) + ( 5 \% \times {A}_{QC} + 5 \% \times {A}_{QMS} 5 \% \times {A}_{Mfg} )$$

Where:
   Cᵢ=Score of checklist item i
   A_{QC}, A_{MFG}, A_{QMS}: % Automation score for respective function

In an embodiment, the present invention provides a method for evaluating the data integrity compliance of a pharmaceutical manufacturing site by determining the CALCULUS™ score, wherein the method comprises the steps of:
(i) determining data integrity compliance score (CALCULUS™ score) of a pharmaceutical manufacturing site, which is computed as a sum of a data integrity (DI) score as a first component and a level of automation score as a second component; wherein; the first component is determined using data integrity compliance checklist based on 163 parameters categorized under:
a) Recording and collection of data
b) Original record/True copy
c) Excluding data,
d) Data Processing
e) Data transfer/migration
f) Data Governance
g) Data Integrity Risk Assessment (DIRA)
h) Computerized System transactions
i) Computerized system User access/System Administrator role
j) Audit Trail
k) Electronic Signatures
l) Data review and approval
m) Data Retention
n) Back up and Archive
o) File structure
p) Validation
q) IT supplier and Service provider
r) Quality Management Systems (QMS)
s) Calibration
t) Quality Control
u) Standalone Systems
v) Trainings
w) Manufacturing process;
wherein the first component or DI score is computed as 85% of the total checklist score;
wherein the second component for measuring the data integrity compliance is related to internal need for increasing and elevating automation to reduce possibility of errors, based on the level of automation at site comprising scoring out of 5% for Quality Control, 5% for Quality Management System and 5% for Manufacturing;
wherein the second component is the sum of the scores for Quality Control, Quality Management System and Manufacturing, out of a total of 15%;
wherein the pharmaceutical manufacturing site is provided a rating of A+, A, B or C based on the DI compliance score (CALCULUS™ score);
wherein,
(i) the rating of A+ is assigned to a site with DI score of >99%;
(ii) the rating of A is assigned to a site with DI score of >85-98.99%;
(iii) the rating of B is assigned to a site with DI score of 70-84.99%;
(iv) the rating of C is assigned to a site with DI score of <70%, and
the qualifying rating for the site as data integrity compliant is A+ or A; with need of improvement for ratings of B or C.

In another aspect, the present invention relates to a method of evaluating the audit readiness of a pharmaceutical manufacturing site, wherein the outcome of the method is determined in the form of a rating obtained via a graphical representation, referred to as PREDICT™ rating which method comprises evaluation of three core elements:
1. A System Score calculated using 132 parameters across six GMP systems in alignment with regulatory inspection models such as USFDA's Six-System approach.
2. A SME Readiness Score representing the site's subject matter expert preparedness during audits.
3. A Data Integrity Compliance Score derived from CALCULUS™.

The PREDICT™ rating provides insight into probable audit outcomes and helps in aligning site focus with the expectation of the audit outcome.

The component PREDICT™ of the present invention is explained herein in detail: PREDICT™ is used for predicting audit or inspection outcome at pharmaceutical manufacturing sites, wherein measurement is based on intangible data, and the outcome is useful to interpret the probable outcome of regulatory inspections at a site.

The predicted outcome comprises of evaluation of three elements;
(i) first element or System score being assessment of GMP system compliance via check points based on 132 vital parameters,
(ii) second element being SME readiness, which integrates aspects of SME readiness at pharmaceutical manufacturing site, and
(iii) third element being data integrity compliance, CALCULUS™ score of pharmaceutical manufacturing site.

System score is designed on USFDA's six system approach. The score helps to assess gaps within six systems at the site. The assessment is done by corporate quality personnel based on corporate audits, customer audits and regulatory inspections. The weightage to checkpoints is allocated based on patient safety, product quality and/or regulatory compliance.

The method of evaluating audit readiness of a pharmaceutical manufacturing site, wherein the outcome of the method is determined in the form of a rating obtained via a graphical representation, referred to as PREDICT™ rating comprises the steps of:

### A. System Score Evaluation:

The assessment covers six categories/systems:
(i) Facility And Equipment System,
(ii) Laboratory Control System,
(iii) Material System,
(iv) Packaging and Labelling System,
(v) Production System, and
(vi) Quality Management Systems;
each being evaluated independently, with equal weightage assigned to all systems. Within these systems, compliance is assessed based on 132 parameters categorized by their relevance to patient safety, product quality and compliance and accordingly the level is defined in TABLE 2 and TABLE 3 as follows:

TABLE 2

| Level | Category | Min Score |
|---|---|---|
| Level 1 | Patient Risk | 3 |
| Level 2 | Product Quality Risk | 2 |
| Level 3 | Compliance Risk | 1 |

TABLE 3
Score Definition

| 1 | 2 | 3 | 4 |
|---|---|---|---|
| Non- | Partially | Compliant with | Highly |
| Compliant | compliant with | scope of | compliant |
| | gaps | improvement | |

TABLE 4 below provides information on the 132 parameters/subsystems used in calculation of the system score, along with category/system, level and weightage information for each parameter.

TABLE 4

| S. no | Parameter/Subsystem | Category/System | Level | Weightage |
|---|---|---|---|---|
| 1. | Equipment cleaning & sanitisation | Facility And Equipment | I | 22% |
| | | System | | |
| 2. | Prevention of cross-contamination, | Facility And Equipment | I | 23% |
| | isolation and containment | System | | |
| 3. | Air handling systems | Facility And Equipment | II | 5% |
| | | System | | |
| 4. | Appropriate use of equipment | Facility And Equipment | II | 5% |
| | operations substances, (lubricants, | System | | |
| | coolants, refrigerants, etc.) contacting | | | |
| | products/containers/etc | | | |
| 5. | Calibration program | Facility And Equipment | II | 5% |
| | | System | | |
| 6. | Clean rooms control, maintenance, & | Facility And Equipment | II | 5% |
| | cleaning | System | | |
| 7. | Control system for implementing | Facility And Equipment | III | 1% |
| | changes in the equipment | System | | |
| 8. | Documented investigation into any | Facility And Equipment | III | 1% |
| | unexpected discrepancy | System | | |
| 9 | Environmental zoning | Facility And Equipment | II | 5% |
| | | System | | |
| 10. | Equipment surfaces should not be | Facility And Equipment | II | 5% |
| | reactive, additive, or absorptive | System | | |
| 11. | Facilities Maintenance | Facility And Equipment | II | 5% |
| | | System | | |
| 12. | Installation Qualification (IQ), | Facility And Equipment | III | 1% |
| | Operational Qualification (OQ), and | System | | |
| | Performance Qualification (PQ) of | | | |
| | facilities, utilities and equipment | | | |
| 13. | Information Technology (IT) GMP, | Facility And Equipment | II | 5% |
| | validation and security | System | | |
| 14. | Planned preventive maintenance | Facility And Equipment | III | 1% |
| | program | System | | |
| 15. | Adequacy of equipment design, size, | Facility And Equipment | III | 1% |
| | and location | System | | |
| 16. | Design guidelines | Facility And Equipment | III | 1% |
| | | System | | |
| 17. | Equipment identification practices | Facility And Equipment | III | 1% |
| | | System | | |
| 18. | Facilities Cleaning | Facility And Equipment | III | 1% |
| | | System | | |
| 19. | Layout & drawings | Facility And Equipment | III | 1% |
| | | System | | |
| 20. | Lighting, potable water, washing and | Facility And Equipment | III | 1% |
| | toilet facilities, sewage and refuse | System | | |
| | disposal | | | |
| 21. | Pest control | Facility And Equipment | III | 1% |
| | | System | | |
| 22. | Sanitation of the building | Facility And Equipment | III | 1% |
| | | System | | |
| 23. | Technical / Design files | Facility And Equipment | III | 1% |
| | | System | | |
| 24. | Zone Ownership | Facility And Equipment | III | 1% |
| | | System | | |
| 25. | Laboratory methods, standards and | Laboratory Control System | II | 5% |
| | controls | | | |
| 26. | Qualification & Validation of all QC | Laboratory Control System | I | 7% |
| | methods | | | |
| 27. | Qualification of all Laboratory | Laboratory Control System | I | 7% |
| | Equipment | | | |
| 28. | Raw data definition, standards, control | Laboratory Control System | I | 7% |
| | & verification | | | |
| 29. | Reference standards management | Laboratory Control System | I | 7% |
| 30. | Retained samples, requirements and | Laboratory Control System | II | 5% |
| | management | | | |
| 31. | Specifications, standards, and | Laboratory Control System | I | 7% |
| | sampling plans | | | |
| 32. | Stability testing | Laboratory Control System | I | 7% |
| 33. | Acceptance Activities and in-process | Laboratory Control System | II | 5% |
| | release | | | |
| 34. | Adequacy of equipment and facility | Laboratory Control System | II | 5% |
| | for intended use | | | |
| 35. | OOS & Out of Trend (OOT) | Laboratory Control System | I | 7% |
| | Investigations | | | |
| 36. | Sample handling, storage, and | Laboratory Control System | II | 5% |
| | integrity | | | |
| 37. | Test data review and authorization | Laboratory Control System | III | 3% |
| 38. | Validation & security of computerized | Laboratory Control System | II | 5% |
| | or automated processes | | | |
| 39. | Adequacy of staffing for laboratory | Laboratory Control System | III | 3% |
| | operations | | | |
| 40. | Calibration program | Laboratory Control System | II | 5% |
| 41. | Equipment List | Laboratory Control System | III | 3% |
| 42. | Maintenance program | Laboratory Control System | III | 3% |
| 43. | Method validation policy and | Laboratory Control System | III | 3% |
| | standards | | | |
| 44. | Trending, reporting, and statistical | Laboratory Control System | III | 3% |
| | quality control | | | |
| 45. | Bill of Materials | Material System | II | 3% |
| 46. | Environmentally controlled storage | Material System | I | 8% |
| | conditions | | | |
| 47. | Finished product distribution records | Material System | I | 8% |
| | by lot | | | |
| 48. | Identification & Quality Status | Material System | I | 8% |
| 49. | Lot traceability of components | Material System | I | 8% |
| | making up a batch | | | |
| 50. | Purified Water system control | Material System | I | 8% |
| 51. | Specifications and acceptance testing | Material System | I | 8% |
| | of raw materials | | | |
| 52. | Active Pharmaceutical Ingredient | Material System | II | 3% |
| | (API), excipient, reagent and | | | |
| | reference material control | | | |
| 53. | Control of quarantine goods | Material System | II | 3% |
| 54. | Control of reject goods | Material System | II | 3% |
| 55. | Expiry dating and retest requirements | Material System | II | 3% |
| 56. | Incoming goods acceptance checks | Material System | II | 3% |
| 57. | Lot numbering control | Material System | II | 3% |
| 58. | Pack Range Control - Artwork, | Material System | II | 3% |
| | components, pack codes | | | |
| 59. | Qualification of cold chain or supply | Material System | II | 3% |
| | chain | | | |
| 60. | Reconciliation | Material System | II | 3% |
| 61. | Reprocessing / rework control | Material System | | 3% |
| 62. | Sampling plan | Material System | II | 3% |
| 63. | Supplier management | Material System | III | 2% |
| 64. | Authority to Destroy & destruction of | Material System | III | 2% |
| | records | | | |
| 65. | Control of Distributors, records, | Material System | III | 2% |
| | Technical Agreements | | | |
| 66. | Control of returned or salvaged goods | Material System | III | 2% |
| 67. | Inventory Management | Material System | III | 2% |
| 68. | Raw materials segregation and | Material System | III | 2% |
| | labelling | | | |
| 69. | Testing or validation of supplier's test | Material System | III | 2% |
| | results for components, containers and | | | |
| | closures | | | |
| 70. | Warehouse controls and First In, First | Material System | III | 2% |
| | Out (FIFO) | | | |
| 71. | Adequate inspection (proofing) of | Packaging and Labelling | I | 10% |
| | incoming labelling | System | | |
| 72. | Control of bulk & unlabelled product | Packaging and Labelling | I | 10% |
| | | System | | |
| 73. | Examination of the labeled finished | Packaging and Labelling | I | 10% |
| | product | System | | |
| 74. | In-process inspection of product | Packaging and Labelling | I | 10% |
| | | System | | |
| 75. | Master Packaging Instructions and | Packaging and Labelling | I | 10% |
| | Records | System | | |
| 76. | QA In-process control checks for | Packaging and Labelling | III | 2% |
| | labeling / packaging operations | System | | |
| 77. | Specifications for packaging & | Packaging and Labelling | II | 6% |
| | labeling materials | System | | |
| 78. | Conformance to tamper-evident | Packaging and Labelling | II | 6% |
| | packaging (TEP) requirements | System | | |
| 79. | Controls and management of | Packaging and Labelling | III | 2% |
| | packaging operations | System | | |
| 80. | Line clearance, inspection, and | Packaging and Labelling | II | 6% |
| | documentation | System | | |
| 81. | Product segregation and labelling | Packaging and Labelling | II | 6% |
| | | System | | |
| 82. | Sampling plan and acceptance | Packaging and Labelling | II | 6% |
| | operations for packaging and labeling | System | | |
| | materials | | | |
| 83. | Storage, issue, inspection, & | Packaging and Labelling | III | 2% |
| | reconciliation of labels and printed | System | | |
| | materials, returns after issue | | | |
| 84. | Validation and security of | Packaging and Labelling | III | 2% |
| | computerized / automated | System | | |
| | labeling/packaging processes | | | |
| 85. | Validation of packaging & labeling | Packaging and Labelling | II | 6% |
| | operation | System | | |
| 86. | Control of issuance of labeling, | Packaging and Labelling | III | 2% |
| | examination of issued labels and | System | | |
| | reconciliation of used labels | | | |
| 87. | Monitoring of printing devices | Packaging and Labelling | III | 2% |
| | | System | | |
| 88. | Physical/spatial separation between | Packaging and Labelling | III | 2% |
| | different labeling and packaging lines | System | | |
| 89. | Component cleaning validation | Production System | I | 6% |
| 90. | Dispensary operations | Production System | I | 6% |
| 91. | Equipment cleaning & use logs | Production System | I | 6% |
| 92. | In process controls | Production System | II | 4% |
| 93. | In-process and final product | Production System | I | 6% |
| | specifications | | | |
| 94. | Justification and consistency of in- | Production System | II | 4% |
| | process specifications and drug | | | |
| | product final specifications | | | |
| 95. | Key manufacturing processes | Production System | II | 4% |
| 96. | Master Manufacturing Instructions | Production System | I | 6% |
| | and Records | | | |
| 97. | Process validation | Production System | I | 6% |
| 98. | Process validation (including | Production System | I | 6% |
| | Computer System Validation (CSV | | | |
| | and security of computerized | | | |
| | /automated processes) | | | |
| 99. | Validation of homogeneity | Production System | I | 6% |
| 100. | Validation of shelf life (stability | Production System | I | 6% |
| | testing) | | | |
| 101. | Adequate procedure and practice for | Production System | II | 4% |
| | charge-in of components | | | |
| 102. | Development products manufacture | Production System | II | 4% |
| | and controls (Technology Transfer) | | | |
| 103. | Environmental monitoring | Production System | II | 4% |
| 104. | Gowning regimes and requirements | Production System | III | 3% |
| 105. | Personnel entry qualification | Production System | III | 3% |
| 106. | Personnel hygiene & medical fitness | Production System | III | 3% |
| 107. | Pre-process checks, line clearance, & | Production System | II | 4% |
| | equipment cleaning | | | |
| 108. | Yield calculations and acceptance | Production System | II | 4% |
| | limits at critical process stages | | | |
| 109. | Control of microbiological spoilage | Production System | III | 3% |
| 110. | Facility cleaning validation | Production System | II | 4% |
| 111. | Identity of equipment contents, phase | Production System | III | 3% |
| | of manufacture/status | | | |
| 112. | Process descriptions (by process) | Production System | III | 3% |
| 113. | Adverse Drug Event (ADE) | Quality Management | I | 5% |
| | Management | Systems | | |
| 114. | Annual Product Review, controls | Quality Management | III | 2% |
| | charts and summary | Systems | | |
| 115. | Batch Manufacturing Records | Quality Management | I | 5% |
| | (BMR)/Batch Packaging Records | Systems | | |
| | (BPR) reviews, approval, archival and | | | |
| | retrieval | | | |
| 116. | Customer complaint management | Quality Management | I | 5% |
| | | Systems | | |
| 117. | Management of Corrective Action and | Quality Management | I | 5% |
| | Preventive Action (CAPA) - Field | Systems | | |
| | Alert Report (FAR), FAR closure and | | | |
| | concomitant CAPA) | | | |
| 118. | Non-conforming materials, root cause | Quality Management | I | 5% |
| | investigation and impact assessment | Systems | | |
| 119. | Previous regulatory observation | Quality Management | I | 5% |
| | closure, Establishment Inspection | Systems | | |
| | Report (EIR) and Regulatory | | | |
| | compliance | | | |
| 120. | Recall Management | Quality Management | I | 5% |
| | | Systems | | |
| 121. | Risk assessment and Mitigation Plans | Quality Management | I | 5% |
| | | Systems | | |
| 122. | Stability programme management | Quality Management | I | 5% |
| | | Systems | | |
| 123. | Technology transfer | Quality Management | I | 5% |
| | | Systems | | |
| 124. | Validation management including | Quality Management | III | 2% |
| | Validation Master Plan (VMP) and | Systems | | |
| | QA peer review | | | |
| 125. | Change control (In plant modification, | Quality Management | II | 12% |
| | material handling, packaging & | Systems | | |
| | labelling etc.) | | | |
| 126. | Deviation Management (product, | Quality Management | II | 12% |
| | process & utilities) | Systems | | |
| 127. | Product disposition | Quality Management | II | 12% |
| | | Systems | | |
| 128. | Quality audits and auditing | Quality Management | III | 2% |
| | | Systems | | |
| 129. | Documentation management, Record | Quality Management | III | 2% |
| | management & Archive - check for | Systems | | |
| | data traceability and real time | | | |
| | recordings, alignment of e-copies and | | | |
| | respective hard copies generated | | | |
| 130. | Management Review and Escalation | Quality Management | III | 2% |
| | procedure | Systems | | |
| 131. | Quality Planning | Quality Management | III | 2% |
| | | Systems | | |
| 132. | Training & Qualification Management | Quality Management | III | 2% |
| | | Systems | | |

Ratings are provided based on compliance status of the sites. Each subsystem is assessed for its compliance. The subsystem score is calculated as per following formula:

Subsystem score=Rating for subsystem×Weightage of subsystem

### System Score Calculation:

The scores of all parameters within each system are summed to determine the subsystem score.
The total system score is obtained by summing the subsystem scores:

$$System Score = {\sum }_{i = 1}^{6} Subsystem {Score}_{i}$$

The maximum possible score for a site is 24 (six systems×maximum score of 4 per system). The interpretation of the score card is provided in TABLE 5 below:

TABLE 5

| System | |
|---|---|
| Scores | Interpretation |
| >20 | Highly compliant sites, high possibility of zero 483, subject to excellent |
| | inspection handling |
| 17-20 | Compliant sites with scope to improve. High possibility of less than or equal to |
| | three 483 s subject to inspection handling |
| 14-16 | Partially compliant sites, high possibility of more than three 483 s subject to |
| | inspection handling |
| <14 | Non-compliant sites, possibility of grave regulatory action. |

### B. SME Readiness Score:

Subject Matter Experts (SMEs) are assessed and scored by the corporate quality team based on their performance during corporate audits, customer audits, and regulatory inspections. These scores are further validated by the Chief Quality Officer.

The scoring criteria for SMEs are clearly defined as follows:
0: Significant changes required in SMEs across all levels, including quality.
1: A few critical SMEs in quality (audit-facing roles) need replacement.
2: A few critical SMEs in other systems (audit-facing roles) need replacement.
3: Challenges exist with 1-2 SMEs (audit-facing roles) but can be resolved.
4: Gaps in quality SMEs (audit-facing roles) can be addressed through training.
5: Gaps in other SMEs (audit-facing roles) can be addressed through training.
6: SMEs (primarily in quality) need extensive audit-facing training but have the required knowledge.
7: SMEs (mainly but not limited to quality) require significant strategic audit-facing training.
8: SMEs (mainly but not limited to quality) require minimal audit-facing training.
9: SMEs (mainly but not limited to quality) need only occasional direction or guidance during audits to defend regulatory cases.
10: SMEs (mainly in quality) are self-reliant and can favourably influence inspections due to strong regulatory knowledge and negotiation skills.

During an audit, each SME is evaluated using this scoring rubric. The individual SME scores are then aggregated, and an average SME score is calculated to represent the site's overall SME readiness. The formula for this is

$$SME Score = \frac{{\sum }_{i = 1}^{n} Individual SME {Scores}_{i}}{n}$$

where n is the total number of SMEs evaluated.

This systematic approach ensures an objective and quantifiable assessment of SME readiness.

### C. CALCULUS™ Score:

The previously computed data integrity compliance, CALCULUS™ Score is directly used as the third component in PREDICT™

### Composite Predictive Model

Each of the above three components: System Score, SME Readiness Score, and CALCULUS™ Score is mapped onto a three-axis visual grid to simulate the site's audit readiness. The combined result helps anticipate the inspection outcome and flags risk levels. The outcome in the form of PREDICT™ rating is determined by compilation of System score, SME readiness score and CALCULUS™ score of respective sites via graphical plotting where,
The System Score is plotted on the X-axis,
The Y-axis value is derived by interpolating the CALCULUS™ Score and SME Score using the following formula:

$$Y = \frac{DI + \frac{( SME - SMEmin ) ( DImax - DImin )}{SMEmax - SMEmin} + DImin}{2}$$

### Where:

SMEₘᵢₙ and SMEₘₐₓ are the minimum and maximum SME readiness scores, respectively,
DIₘᵢₙ and DIₘₐₓ are the minimum and maximum CALCULUS™ scores, respectively.
Minimum System Score is 10 and Maximum is 24,
Minimum SME Score (SMEₘᵢₙ) is 3 and Maximum (SMEₘᵢₙ) is 11, and
Minimum CALCULUS™ Score (DIₘᵢₙ) is 65 and Maximum (DIₘₐₓ) is 84.

The above graphical representation uses an approach ensuring that the SMVE and CALCULUS™ scores are equitably represented, yielding a balanced Y-axis value that reflects both factors.

The PEDICT™ rating is given based on the graphical zone in which the site would lie. Interpretation of sites outcome and rating is depicted in below TABLE 6:

TABLE 6
Predict ™ Interpretation

| Predict | Predict | Observation | Number & | |
|---|---|---|---|---|
| Outcome | Rating | Severity | category | Regulations |
| Red(Poor) | 1 | High | Double digit | FDA |
| | | | WL/483 | EU/MHRA |
| | | | Critical/RUA | |
| Amber | 2 | Moderately | 7-10 483, Critical: | FDA |
| (Moderately | | High | 1-2, Major: | EU/MHRA |
| Poor) | | | Several or | |
| | | | category A type | |
| Yellow | 3 | Moderate | ≤7 483, | FDA |
| (Moderate) | | | No critical, | EU/MHRA |
| | | | Major: Several or | |
| | | | category A type | |
| Light Green | 4 | Low | ≤3 483, | FDA |
| (Good) | | | No critical, Major: | EU/MHRA |
| | | | Few | |
| Dark Green | 5 | Continuous | 0-2 483 | FDA |
| (Excellent) | | Improvement | No critical/No | EU/MHRA |
| | | or non- | Major | |
| | | systemic | | |
| | | gaps | | |

In an embodiment, the present invention provides a method of evaluating audit readiness of a pharmaceutical manufacturing site by determining the PREDICT™ rating according to claim 1, wherein the method comprises the steps of:
(i) determining first element or system score being assessment of Good Manufacturing Practice (GMP) system compliance via check points based on six systems comprising 132 parameters categorized into three levels namely Patient Risk (Level-I), Product Quality Risk (Level-II) and Compliance Risk (Level-III), wherein the six systems are:
   1. Facility And Equipment System,
   2. Laboratory Control System,
   3. Material System,
   4. Packaging and Labelling System,
   5. Production System, and
   6. Quality Management Systems;
(ii) determining the second element being assessment of subject matter expert (SME) by corporate auditors, which integrates aspects of subject matter expert readiness at pharmaceutical manufacturing sites, and
(iii) determining the third element being data integrity compliance score, CALCULUS™ score of a pharmaceutical manufacturing site; wherein the predicted outcome is determined by compilation of system score, SME score and CALCULUS™ score of respective site graphically and the interpretation of predicted outcome and score is interpreted to provide the PREDICT™ rating; wherein,
   (a) if the predicted outcome is red (poor), predict rating is 1, then the severity of observation is high and double-digit warning letters could be expected from regulatory body during audit,
   (b) if the predicted outcome is amber (moderately poor), predict rating is 2, then the severity of observation is moderately high and 7-10 warning letters could be expected from regulatory body during audit,
   (c) if the predicted outcome is yellow (moderate), predict rating is 3, then the severity of observation is moderate and <7 warning letters could be expected from regulatory body during audit,
   (d) if the predicted outcome is light green (good), predict rating is 4, then the severity of observation is low and <3 warning letters could be expected from regulatory body during audit, and
   (e) if the predicted outcome is dark green (excellent), predict rating is 5, then the severity of observation is indicative of continuous improvement or non-systemic gaps and 0-2 warning letters could be expected from regulatory body during audit.

In a third aspect, the present invention relates to a method of measuring the quality health of a pharmaceutical manufacturing site, wherein the quality is visually measured on a level meter, referred to as SENSOR™, as a dynamic site score on a scale from 0 to 5, which is determined by consolidating eleven critical quality indicators, each assigned a predefined rationalized weightage, wherein said score. is interpreted to classify sites by their health status, required level of remediation and associated compliance risk.

The component SENSOR™ of the present invention is explained herein in detail: The SENSOR™ score measured on a level meter, is developed to provide a holistic, quantifiable, and predictive assessment of a pharmaceutical manufacturing site's regulatory health and operational maturity. It serves as a single-point, real-time decision-support tool for senior leadership to evaluate, compare, and prioritize quality performance across sites.

The site SENSOR™ score is computed using eleven core quality criteria, carefully selected from globally accepted pharmaceutical regulations and best practices, including but not limited to WHO, US-FDA, EMA, PIC/S, and ISPE guidelines. These eleven parameters holistically represent a pharmaceutical site's quality framework, spanning systems, processes, data integrity, and people capability.

The SENSOR™ score integrates:
the CALCULUS™ Score,
the Audit Score computed via the PREDICT™ module comprises of System Score and SME Score, and
a set of nine critical real-time quality indicators related to regulatory exposure, operational compliance, and people capability.

The method of measuring the quality health of a pharmaceutical manufacturing site, wherein the quality is visually measured on a level meter, SENSOR™, a dynamic site score on a scale from 0 to 5, is determined by eleven critical quality indicators:
Audit Score (System score+SME Score)
Data integrity compliance score (CALCULUS™ Score)
Product Quality Complaints
Invalidated Out of Specification (OOS)
Corrective Action and Preventive Action (CAPA) closure rate
Process OOS or Right First Time (RFT)
Investigation closure rate
Stability On Time in Full (OTIF)
Change Control closure rate
Deviation Closure rate
Standard Operating Procedure Validity

### 1. Audit Score (System Score+SME Score)

The audit score is derived through a detailed checklist also termed as system score representing key six system vital for operating and maintaining the quality of the products manufactured at a pharmaceutical manufacturing site. These systems include Quality Management Systems, Facility and Equipment System, Material System, Production System, Packaging and Labelling System, and Laboratory Control System, as detailed in PREDICT™.
All the above system were evaluated through internal audits and the outcome of the audit is summarized through a detailed checklist covering 132 checkpoints or parameters. The outcome represents the site readiness for the audit and areas for improvement. This helps the site on allocation of resources and efforts in focused manner to address the gaps if any. This System Score helps in informed decision making to improve the health of the site. Further, the SME score is also determined based on the criteria mentioned in previous aspect of PREDICT™
A detailed explanation of the calculation of System score and SME Score is mentioned in previous aspect of PREDICT™.

### 2. Data Integrity Compliance Score (CALCULUS™ Score)

The Data Integrity compliance score is derived through a detailed checklist of 163 parameters covering requirements from diverse regulatory guidelines and integration with organizational and technical controls to form a comprehensive data governance framework for Data Integrity. Organizational controls include policies, procedures, and training, etc. while technical controls involve system configurations, access management, and audit trails etc.
The checklist executed on an annual basis for all sites to evaluate any gaps at the site. The outcome represents the site compliance on Data Integrity requirements and areas for improvement.
Inclusion of automation level in Quality Control, Quality Assurance and Manufacturing processes in the Data Integrity compliance score, promote technology adoption and improve controls on Data Integrity. Encouragement of automation reduces dependence on manual controls, reduce manual errors, Data Integrity risks, and enhance efficiency.
A detailed explanation of the calculation of DI compliance score is mentioned in previous aspect of CALCULUS™

### 3. Product Quality Complaints

Quality Complaints have a potential to adversely affect the health and safety of the patients and goodwill of the company and/or product brand. It is preferred to have real time visibility to track each complaint through its life cycle-from its recording and initiation of the investigation, reporting, and closure.
The proposed methodology offers a comprehensive solution for real time visibility of complaints per batches dispatched. The method leverages data analytics to recognize patterns in recurring complaints, providing actionable insights for root cause analysis and facilitating the implementation of preventive measures to mitigate future issues. This systematic approach not only enhances operational efficiency but also significantly improves customer satisfaction and trust in the company's products.
The method has a provision of negative marking for each major/critical/recurring complaint received, ensuring that these are addressed promptly and holistically to maintain product quality and regulatory compliance.

### 4. Invalidated Out of Specification (OOS)

Invalidated Out-of-Specification rate (IOOSR) act as an indicator of laboratory operation and performance. IOOSR is defined as the number of OOS test results for lot release and long-term stability testing invalidated by the reporting establishment due to an aberration of the measurement process divided by the total number of lot release and long-term stability OOS test results in the current reporting timeframe. This metric helps in identifying any area of improvement in the laboratory performance.

### 5. Corrective Action and Preventive Action (CAPA) Closure Rate

The timely closure of Corrective and Preventive Actions (CAPA) at pharmaceutical manufacturing sites is crucial for maintaining compliance, ensuring product quality, and patient safety. CAPA processes are designed to identify, address, and prevent the recurrence of issues that could affect the product quality, process, or GxP compliance. On-time completion of CAPA not only demonstrates a site's commitment to adhering to regulatory standards set by agencies but also mitigate the risk or failures. Timely CAPA closure fosters a culture of continuous improvement and enhances operational efficiency. Delays in closing CAPAs can lead to regulatory scrutiny, potential financial impact, and loss of reputation, making it imperative for manufacturing sites to prioritize and streamline their CAPA processes.
This scoring is derived by calculating % CAPA closed on time divided by total CAPA due for closure in the evaluation period.

### 6. Process OOS or Right First Time (RFT)

The process OOS monitors the Right First Time (RFT) processes in pharmaceutical manufacturing, with a particular focus on the number of Out of Specification (OOS) versus the number of batches released. This process is specifically developed to provide real-time tracking and analysis of OOS occurrences, allowing for precise identification and rectification of failures. By correlating OOS data with the number of batches released, the process offers comprehensive insights into process performance and reliability. It helps in pinpointing the root causes of inefficiencies and ensures that corrective actions are effectively implemented. This proactive monitoring capability significantly enhances batch quality, minimizes production delays, and supports regulatory compliance, thereby promoting overall operational excellence in pharmaceutical manufacturing.

### 7. Investigation Closure Rate

Timely closure of investigations in pharmaceutical manufacturing sites is essential for ensuring regulatory compliance, maintaining product quality, and protecting patient safety. Investigations are initiated to examine deviations, complaints, incidents, or any out-of-specification results that may impact products quality. Prompt and thorough investigation closure helps to quickly identify root causes, implement corrective actions, and prevent recurrence, thereby risk mitigation. On-time completion of investigations not only demonstrates a site's commitment to adhering to regulatory standards set by agencies but also mitigate the risk or failures. Timely investigation closure fosters a culture of continuous improvement and enhances operational efficiency. Delays in closing investigations can lead to regulatory scrutiny, potential financial impact, and loss of reputation, making it imperative for manufacturing sites to prioritize and streamline their CAPA processes. This scoring is derived by calculating % investigations closed on time divided by total Investigations due for closure in the evaluation period.

### 8. Stability On Time in Full (OTIF)

Timely completion of stability analysis in pharmaceutical manufacturing is vital for ensuring the safety, efficacy, and shelf-life of medications. Stability studies are conducted to evaluate how the quality of a drug substance or product varies over time under the influence of various environmental factors like temperature, humidity, and light. By adhering to predetermined testing schedules and completing stability analyses on time, pharmaceutical manufacturing sites can establish the product's shelf-life, storage conditions, and appropriate packaging requirements. This timely completion also enables manufacturing sites to promptly detect any potential degradation or changes in the product's attributes, ensuring that only safe and effective medications reach patients. Furthermore, meeting stability analysis timelines supports regulatory compliance with agencies, enhancing product registration and market approval processes. In summary, timely completion of stability studies is crucial for maintaining product quality, regulatory compliance, and patient safety in the pharmaceutical industry.
This scoring is derived by calculating % Stability analysis closed on time divided by total stability analysis due for closure in the evaluation period.

### 9. Change Control Closure Rate

The timely closure of change controls in pharmaceutical manufacturing sites is essential for maintaining product quality, regulatory compliance, and operational efficiency. Change controls are systematic approaches used to manage any alterations in manufacturing processes, equipment, or procedures to ensure that such changes do not negatively impact product quality or patient safety. Prompt closure of change controls ensures that all modifications are thoroughly evaluated, documented, and implemented within a controlled and timely manner, minimizing the risk of production delays, non-compliance, and potential quality risks. It also facilitates continuous improvement by allowing the company to adapt swiftly to technological advancements, regulatory updates, and market demands. Timely change control closure demonstrates a company's commitment to rigorous quality management practices and regulatory adherence, thereby fostering trust with regulatory bodies, customers, and stakeholders. Delays in this process can lead to operational inefficiencies, increased costs, and potential regulatory scrutiny, highlighting the critical importance of effective change control management in the pharmaceutical industry. This scoring is derived by calculating % change controls closed on time divided by total change controls due for closure in the evaluation period.

### 10. Deviation Closure Rate

The timely closure of deviations in pharmaceutical manufacturing sites is critical for maintaining product quality, ensuring regulatory compliance, and safeguarding patient safety. Deviations, which are departures from standard operating procedures or expected results, can signal underlying issues that may impact the integrity and efficacy of products. Prompt resolution of these deviations allows for quick identification and rectification of root causes, preventing potential problems from escalating. On-time completion of deviations not only demonstrates a site's commitment to adhering to regulatory standards set by agencies but also mitigate the risk or failures. Timely deviation closure fosters a proactive quality management culture, enhances operational efficiency, and builds trust with regulatory bodies and consumers. Delays in addressing deviations can lead to increased scrutiny, financial impact, and reputational damage, making it essential for pharmaceutical companies to prioritize and streamline their deviation management processes.
This scoring is derived by calculating % deviations closed on time divided by total deviations due for closure in the evaluation period.

### 11. Standard Operating Procedure Validity

The timely periodic review of Standard Operating Procedures (SOPs) is fundamental for ensuring their validity and effectiveness in pharmaceutical manufacturing. Regular reviews guarantee that SOPs remain up to date with current regulatory requirements, technological advancements, and best practices, thereby maintaining operational consistency and compliance. This process helps identify and incorporate necessary updates, rectify any outdated information, and ensure that all procedures are clear and accurate. Timely reviews also support continuous improvement initiatives by integrating feedback from audits, inspections, and day-to-day operations. Ensuring the validity of SOPs enhances the quality and safety of pharmaceutical products, reduces the risk of errors and deviations, and fortifies the company's commitment to high standards of operational excellence. Neglecting periodic SOP reviews can lead to compliance gaps, inefficiencies, and potential regulatory observations, underscoring the critical importance of maintaining current and accurate SOPs.

### Rationale for Weightage:

The weightage assignment for the parameters for evaluating the quality health of a pharmaceutical manufacturing site can be rationalized based on their impact on product quality, compliance, and overall operational efficiency. A detailed rationale for each parameter is provided herein below:

### 1. Data Integrity Compliance Score (12%)

Ensures that all the GxP data is accurate, complete, and reliable.
Essential for regulatory compliance and maintaining product quality.
High impact on overall compliance and risk management.

### 2. Product Quality Complaints (10%)

Reflects customer satisfaction and product performance in the market.
Directly related to the quality of the product and the effectiveness of the quality management system.
Important for continuous improvement and customer trust.

### 3. Invalidated OOS (Out of Specification) (8%)

Measures the instances where test results fall outside the specified limits but are invalidated upon further investigation.
Critical for understanding the robustness of the quality control process.
Helps identify potential weaknesses in the testing process.

### 4. CAPA (Corrective and Preventive Action) Closure Rate (12%)

Indicates the efficiency and effectiveness of the CAPA system.
Important for continuous improvement and preventing recurrence of issues.
Reflects the organization's commitment to addressing and resolving quality issues.

### 5. Process OOS or RFT (8%)

Measures the percentage of processes that are completed correctly the first time without rework.
Although important, it is weighted lower because other parameters directly address the quality and compliance aspects more significantly.

### 6. Investigations Closure Rate (7%)

Measures the timely closure of investigations related to deviations, complaints, and OOS results.
Critical for maintaining compliance and ensuring timely resolution of quality issues.
Reflects the effectiveness of the quality management system.

### 7. Stability OTIF (On-Time In-Full) (10%)

Measures the adherence to the stability study schedule.
Important for ensuring product stability over its shelf life.
Reflects the organization's ability to maintain consistent product quality.

### 8. Change Control Closure (8%)

Measures the timely implementation of changes in processes, equipment, or procedures.
Critical for ensuring that changes do not adversely affect product quality.
Reflects the organization's ability to manage and control changes effectively.

### 9. Deviation Closure (12%)

Measures the timely closure of deviations from standard procedures.
Important for maintaining compliance and preventing recurrence of issues.
Reflects the organization's commitment to resolving quality issues promptly.

### 10. SOP (Standard Operating Procedure) Validity (3%)

Ensures that all SOPs are current and valid.
Although important, it is weighted lower because it supports the overall quality system rather than directly impacting product quality.

### 11. Audit Score (10%)

Measures the findings from internal and external audits.
Assigned the highest weightage due to its direct impact on regulatory compliance and the overall effectiveness of the quality management system.
Reflects the organization's adherence to regulatory requirements and industry standards.

In summary, the weightage is assigned based on the criticality of each parameter to maintaining product quality, regulatory compliance, and operational efficiency. Parameters directly impacting these areas are given higher weightage, while supporting parameters are given relatively lower weightage.

The SENSOR™ score is visually represented through a color-coded level meter that enables risk-stratified decision-making. Each zone in the meter corresponds to a defined level of compliance risk and indicates the required focus level-ranging from routine governance to immediate executive intervention. This structured visualization aids in aligning strategic actions with site-specific compliance health and operational maturity.

In an embodiment, the level meter is demarcated into 5 colored regions corresponding to the score and indicative of Site Health, selected from:
1) Score of 0-1 corresponding to red region indicating that the Site Health has Chronic Serious Illness,
2) Score of 1-2 corresponding to orange region indicating that the Site Health has Chronic Non-serious Illness,
3) Score of 2-3 corresponding to yellow region indicating that the Site Health has Acute Frequent Symptoms,
4) Score of 3-4 corresponding to light green region indicating that the Site Health has Acute In-frequent Symptoms, and
5) Score of 4-5 corresponding to green region indicating that the Site is Healthy.

In another embodiment, the level meter is demarcated into 5 colored regions corresponding to the score and indicative of Cure, selected from:
a) Score of 0-1 corresponding to red region indicating that the Cure required is Intensive Care,
b) Score of 1-2 corresponding to orange region indicating that the Cure required is Focused Treatment post Diagnosis,
c) Score of 2-3 corresponding to yellow region indicating that the Cure required is Symptomatic cure followed by eradication via Diagnosis,
d) Score of 3-4 corresponding to light green region indicating that the Cure required is Symptomatic cure, and
e) Score of 4-5 corresponding to green region indicating that the Cure required is Prophylactic measures to Sustain and Routine Checks.

In yet another embodiment, the level meter is demarcated into 5 colored regions corresponding to the score and indicative of Risks and Focus, selected from:
a) Score of 0-1 corresponding to red region indicating that the Risk is Very High and Focus is Immediate,
b) Score of 1-2 corresponding to orange region indicating that the Risk is High and Focus is High,
c) Score of 2-3 corresponding to yellow region indicating that the Risk is Medium and Focus is High,
d) Score of 3-4 corresponding to light green region indicating that the Risk is Medium and Focus is Medium, and
e) Score of 4-5 corresponding to green region indicating that the Risk is Low and Focus is Medium.

In a further embodiment, the level meter is demarcated into 5 colored regions corresponding to five zones of the decision matrix selected from:
a) Score of 0-1 corresponding to a high-risk, immediate action zone,
b) Score of 1-2 corresponding to a high focus zone,
c) Score of 2-3 corresponding to a moderate focus zone,
d) Score of 3-4 corresponding to a maintenance and sustainability zone, and
e) Score of 4-5 corresponding to a low-risk zone indicating regulatory readiness.

In an embodiment the present invention provides a method of measuring the quality health of a pharmaceutical manufacturing site, wherein the quality is visually measured on a level meter, SENSOR™, a dynamic site score on a scale from 0 to 5, comprising the steps of:
(a) determining eleven critical quality indicators:
   i. Audit Score (sum of System score as determined in step (i) of claim 4 and SME Score as determined in step (ii) of claim 4),
   ii. Data integrity compliance score (CALCULUS™ Score) as determined in claim 2,
   iii. Product Quality Complaints,
   iv. Invalidated Out of Specification (OOS),
   v. Corrective Action and Preventive Action (CAPA) closure rate,
   vi. Process OOS or Right First Time (RFT),
   vii. Investigation closure rate,
   viii. Stability On Time in Full (OTIF),
   ix. Change Control closure rate,
   x. Deviation Closure rate, and
   xi. Standard Operating Procedure Validity
(b) assigning weightages to each of the eleven indicators based on their criticality to compliance, product quality, and patient safety.
(c) rating each indicator on a predefined scale and computing the weighted sum to obtain the SENSOR™ score.
(d) interpreting the SENSOR™ score within a five-zone decision matrix, each zone corresponding to predefined levels of site health, audit risk, and required focus of intervention; wherein, the level meter is demarcated into 5 colored regions corresponding to the score, selected from:
   1) Score of 0-1 corresponding to red region indicates that the Site Health has Chronic Serious Illness, Cure required is Intensive Care, Risk is Very High and Focus required is Immediate,
   2) Score of 1-2 corresponding to orange region indicating that the Site Health has Chronic Non-serious Illness, Cure required is Focused Treatment post Diagnosis, Risk is High and Focus required is High,
   3) Score of 2-3 corresponding to yellow region indicating that the Site Health has Acute Frequent Symptoms, Cure required is Symptomatic cure followed by eradication via Diagnosis, Risk is Medium and Focus required is High,
   4) Score of 3-4 corresponding to light green region indicating that the Site Health has Acute In-frequent Symptoms, Cure required is Symptomatic cure, Risk is Medium and Focus required is Medium, and
   5) Score of 4-5 corresponding to green region indicating that the Site is Healthy, Cure required is Prophylactic measures to Sustain and Routine Checks, Risk is Low and Focus required is Medium.

In an embodiment of the present invention, when the computed scores for CALCULUS™ PREDICT™ or SENSOR™ indicate performance below predefined thresholds, a systematic remediation approach is applied to elevate site compliance health, audit readiness, and operational quality. The methodology not only measures gaps but also prescribes corrective actions aligned with regulatory expectations.

### 1. Data Integrity Compliance Improvement (CALCULUS™)

When the CALCULUS™ score indicates deficiencies in data governance or automation, the following structured interventions are undertaken:
   Targeted Gap Analysis: Reassess checklist items to identify specific weaknesses in technical controls (e.g., audit trail, backup, user access) and organizational controls (e.g., SOP adherence, training). Based on requirement a team from central Data Integrity Compliance or a third-party consultant may be deployed.
   Process Strengthening: Revise or create procedures to ensure compliance with ALCOA+(Attributable, Legible, contemporaneous, Original, Accurate)& ‘+’ (Complete, Consistent, Enduring, Available) principles.
   Technology Enhancement: Implement or increase automation in Quality Control, Manufacturing, and Quality Assurance processes to reduce manual errors and enhance traceability. A long term and short-term action plan for automation can be devised with management.
   Capability Building: Conduct focused staff training to reduce unintentional data errors and reinforce a compliance culture.
   Ongoing Verification: Perform periodic internal data integrity audits to ensure sustained improvement.

### 2. Audit Readiness Enhancement (PREDICT™)

For sites receiving a low PREDICT™ rating, the following corrective actions improve system maturity and inspection preparedness:
   System Compliance Upgrades: Close gaps across the six USFDA quality systems by implementing corrective measures and assigning system-specific owners. Based on requirement a technical support team from corporate Quality or a third-party consultant may be deployed for gap assessment and compliance.
   SME Readiness Development: Identify subject matter experts requiring capability enhancement and provide targeted mock audits, regulatory knowledge sessions, and response training.
   Simulation and Stress Testing: Conduct unannounced mock inspections to test real-time preparedness and document retrieval efficiency.
   Legacy Gap Closure: Review and resolve historical regulatory observations to prevent repeated findings in future audits.

### 3. Operational Quality Reinforcement (SENSOR™)

When a SENSOR™ score falls below the defined benchmark, site-level operational metrics are improved by:
   Prioritizing High-Impact KPIs: Focus on parameters carrying higher weightage such as audit score, CAPA timeliness, and deviation closures.
   Governance Controls: Establish strict monitoring mechanisms and automated dashboards to track closure rates for CAPAs, deviations, and investigations.
   Complaint Management Optimization: Investigate root causes of product quality complaints promptly and implement preventive measures.
   Process Reliability: Minimize OOS/OOT events through improved process validation, equipment maintenance, and operator qualification.
   SOP update: Ensure all SOPs are periodically reviewed, updated, and effectively implemented to maintain procedural compliance. This can be done by deployment of additional resources on temporary basis.

### 4. Integrated Governance and Monitoring

The methodology incorporates a cross-functional governance framework to maintain and enhance site compliance level:
   Establish monthly review of leading indicators to monitor and improve performance.
   Implement real-time scorecards to detect early signs of decline.
   Allocate corporate support and resources where systemic improvements, additional staff, or digital tools are required.

By applying the above remediation strategy, sites demonstrating low CALCULUS™ score, PREDICT™ rating or SENSOR™ score can systematically improve their compliance health, operational reliability, and audit readiness. This structured, weighted and automated methodology ensures that weaknesses are identified, addressed and continuously monitored, thereby aligning site operations with regulatory expectations and corporate quality objectives.

Each of CALCULUS™ score, PREDICT™ rating or SENSOR™ score may be independently evaluated irrespective of the other two components based on the requirement of the pharmaceutical manufacturing site. For instance, CALCULUS™ score may be determined to evaluate the data integrity compliance of a site without the need to determine the PREDICT™ rating or SENSOR™ score. Similarly, the PREDICT™ rating may be determined to evaluate the audit readiness of the site (which involves determining CALCULUS™ score) without the need to determine the SENSOR™ score. Likewise, the quality health of the site may be evaluated by determining the SENSOR™ score.

The innovative methodical approach, detailed categorization, and balanced scoring formula provide a robust framework for maintaining and improving compliance across pharmaceutical manufacturing sites. This process not only ensures compliance with regulatory standards but also promotes the adoption of automation, and latest regulatory requirements leading to more efficient and reliable quality management.

### Examples

### Evaluation of CALCULUS™ Score, PREDICT™ Rating and SENSOR™ Score

### Example 1—Determination of CALCULUS™ Score

The pharmaceutical manufacturing site under consideration was evaluated for data integrity compliance as follows:

TABLE 7
Data Integrity (DI) Score - 163 parameters

| | | Essential/ | | | |
|---|---|---|---|---|---|
| | | Critical | Std | | |
| Category | Checkpoint | parameter | Weightage | Status | Score |
| Recording and | Checking whether an Standard | Essential | 0.40% | Yes | 0.40% |
| collection of | Operating Procedure (SOP) or | | | | |
| data | guideline on Good | | | | |
| | Documentation Practices exists, | | | | |
| | whether it covers aspects of | | | | |
| | ‘ALCOA’ (Attributable, Legible, | | | | |
| | contemporaneous, Original, | | | | |
| | Accurate) & ‘+’ (Complete, | | | | |
| | Consistent, Enduring, | | | | |
| | Available) principle and | | | | |
| | whether they are followed | | | | |
| Recording and | Checking whether an SOP for | Critical | 2.86% | No | 0.00% |
| collection of | Issuance, document control and | | | | |
| data | reconciliation & destruction of | | | | |
| | Blank forms/Batch records, | | | | |
| | paginated logbooks and Lab | | | | |
| | Notebooks exists | | | | |
| Recording and | Checking whether a process of | Critical | 2.86% | Yes | 2.86% |
| collection of | ensuring that always the current | | | | |
| data | copy of SOP is in circulation and | | | | |
| | the obsolete SOPs are | | | | |
| | withdrawn in timely manner | | | | |
| | with appropriate reconciliation | | | | |
| | is in place | | | | |
| Recording and | Checking whether uncontrolled | Essential | 0.40% | Yes | 0.40% |
| collection of | copies are issued and if issued, | | | | |
| data | whether the SOP defines the | | | | |
| | procedure on issuance and | | | | |
| | handling of uncontrolled copies | | | | |
| Recording and | Checking whether issuance of | Critical | 2.86% | Yes | 2.86% |
| collection of | Batch Manufacturing Record | | | | |
| data | (BMR), Batch Packaging | | | | |
| | Record (BPR) and lab notebooks | | | | |
| | is controlled by Quality | | | | |
| | Assurance (QA), Document | | | | |
| | controller or by electronic | | | | |
| | module and whether each page | | | | |
| | is either stamped and signed or | | | | |
| | bears a unique identification | | | | |
| | with page number (auto | | | | |
| | generated) | | | | |
| Recording and | Checking whether there is a | Critical | 2.86% | Yes | 2.86% |
| collection of | proper control over replacement | | | | |
| data | of any page(s) and in times of | | | | |
| | genuine need, whether this is | | | | |
| | done through a validated system | | | | |
| | control and whether replacement | | | | |
| | is recorded adequately | | | | |
| Recording and | Checking whether there is a | Essential | 0.40% | Yes | 0.40% |
| collection of | logbook maintained for issuance | | | | |
| data | of forms, templates or similar | | | | |
| | documents | | | | |
| Recording and | Checking whether all Quality | Essential | 0.40% | Yes | 0.40% |
| collection of | related activities are recorded at | | | | |
| data | the same time when they are | | | | |
| | performed, and whether BMR, | | | | |
| | BPR and Lab notebooks are | | | | |
| | recorded contemporaneously | | | | |
| Recording and | Checking whether the site has a | Essential | 0.40% | Yes | 0.40% |
| collection of | practice of recording data on | | | | |
| data | pieces of paper that will be | | | | |
| | discarded after the data are | | | | |
| | transcribed to a permanent | | | | |
| | laboratory notebook | | | | |
| Recording and | Checking whether there is a | Essential | 0.40% | Yes | 0.40% |
| collection of | practice of use of scribes to | | | | |
| data | record activity on behalf of | | | | |
| | another operator, if such practice | | | | |
| | exists, whether it is verified and | | | | |
| | documented contemporaneously | | | | |
| | or retrospectively with adequate | | | | |
| | justification, and whether this is | | | | |
| | defined as part of procedure | | | | |
| Recording and | Checking whether there exist | Essential | 0.40% | Yes | 0.40% |
| collection of | practices to ensure in routine and | | | | |
| data | confirm that there is no | | | | |
| | backdating, postdating, omitting | | | | |
| | negative data (Out of | | | | |
| | Specification (OOS) or | | | | |
| | eliminating outliers) or releasing | | | | |
| | failing product, manipulation, | | | | |
| | deliberated hidden information | | | | |
| | or missing signatures on any | | | | |
| | Good Manufacturing Practices | | | | |
| | (GMP) or Quality Management | | | | |
| | System (QMS) record | | | | |
| Recording and | Checking whether the team has | Essential | 0.40% | Yes | 0.40% |
| collection of | appropriate level of process | | | | |
| data | understanding and technical | | | | |
| | knowledge of systems used for | | | | |
| | data collection and recording, | | | | |
| | including their capabilities, | | | | |
| | limitations and vulnerabilities | | | | |
| Recording and | Checking whether the system | Essential | 0.40% | Yes | 0.40% |
| collection of | has feature to not obscure | | | | |
| data | previously recorded information | | | | |
| | during record change by | | | | |
| | authorized personnel | | | | |
| Recording and | Checking whether adequate | Essential | 0.40% | Yes | 0.40% |
| collection of | controls exist in case of run | | | | |
| data | abortion in High Performance | | | | |
| | Liquid Chromatography | | | | |
| | (HPLC) systems | | | | |
| Recording and | Checking whether electronic | Essential | 0.40% | No | 0.00% |
| collection of | system (HPLC, QMS | | | | |
| data | application, server) downtime | | | | |
| | are documented and investigated | | | | |
| | for the reason | | | | |
| Recording and | Checking whether key process | Essential | 0.40% | Yes | 0.40% |
| collection of | equipment have controls that | | | | |
| data | measure its operating range of | | | | |
| | critical process parameters and | | | | |
| | whether this range is reviewed | | | | |
| | during batch release decision | | | | |
| Recording and | Checking whether hybrid | Essential | 0.40% | No | 0.00% |
| collection of | systems are used and if used, | | | | |
| data | whether they are clearly | | | | |
| | documented for which data set is | | | | |
| | to be reviewed and retained | | | | |
| Recording and | Checking whether the system | Essential | 0.40% | Yes | 0.40% |
| collection of | has the ability to generate | | | | |
| data | accurate and complete data | | | | |
| | records in both paper and | | | | |
| | electronic format (human | | | | |
| | readable) which could be readily | | | | |
| | available and accessible (if | | | | |
| | requested by national competent | | | | |
| | authorities) | | | | |
| Recording and | Checking whether the system | Essential | 0.40% | Yes | 0.40% |
| collection of | generated report specifies the | | | | |
| data | time zone where this data is used | | | | |
| | across multiple sites | | | | |
| Recording and | Checking whether there exists a | Essential | 0.40% | Yes | 0.40% |
| collection of | practice of retention of | | | | |
| data | incomplete or erroneous forms | | | | |
| | as permanent records along with | | | | |
| | justification of their replacement | | | | |
| | and whether this is governed by | | | | |
| | SOP | | | | |
| Recording and | Checking whether records or | Essential | 0.40% | Yes | 0.40% |
| collection of | procedures are accessible to | | | | |
| data | locations where activities take | | | | |
| | place | | | | |
| Original | Checking whether there exists a | Essential | 0.40% | Yes | 0.40% |
| Record & true | system or procedure of | | | | |
| copy | Handling, Review and Retention | | | | |
| | of original records- | | | | |
| | dynamic(electronic) records and | | | | |
| | static (printed/manual) records | | | | |
| | generated at Site | | | | |
| Original | Checking whether the data (or a | Essential | 0.40% | Yes | 0.40% |
| Record & true | true copy) generated in paper | | | | |
| copy | format is retained in the form of | | | | |
| | scan copies | | | | |
| Original | Checking whether there exists | Essential | 0.40% | Yes | 0.40% |
| Record & true | any data at the site where it is not | | | | |
| copy | feasible to retain in original | | | | |
| | form, and if it such data exists, | | | | |
| | whether the risk assessment is | | | | |
| | documented and whether | | | | |
| | appropriate form of data is | | | | |
| | retained as true copy. | | | | |
| Original | Checking whether a true copy of | Essential | 0.40% | Yes | 0.40% |
| Record & true | the original data is maintained | | | | |
| copy | securely throughout the records | | | | |
| | retention period, whether there | | | | |
| | is a risk assessment performed in | | | | |
| | case of destruction of original | | | | |
| | record and whether this is | | | | |
| | governed by SOP | | | | |
| Original | Checking whether there exists a | Essential | 0.40% | Yes | 0.40% |
| Record & true | process of risk assessment of | | | | |
| copy | data where the data obtained | | | | |
| | requires manual observation to | | | | |
| | record the results of manual | | | | |
| | titration, visual interpretation of | | | | |
| | environmental monitoring plates | | | | |
| | and similar results | | | | |
| Original | Checking whether manual | Essential | 0.40% | Yes | 0.40% |
| Record & true | transcription for above point are | | | | |
| copy | verified by second person or | | | | |
| | validated system | | | | |
| Original | Checking whether the true copy | Essential | 0.40% | Yes | 0.40% |
| Record & true | is stored in different electronic | | | | |
| copy | format to the original record and | | | | |
| | if stored in a different format, | | | | |
| | whether retention of meta data | | | | |
| | and audit trail are ensured | | | | |
| Excluding Data | Checking whether there is a | Essential | 0.40% | Yes | 0.40% |
| | process for exclusion of data and | | | | |
| | if such process exists, whether | | | | |
| | there is a process to demonstrate | | | | |
| | through valid scientific | | | | |
| | justification that the data are not | | | | |
| | representative of the quantity | | | | |
| | measured, sampled or acquired | | | | |
| Data | Checking whether reprocessing | Essential | 0.40% | Yes | 0.40% |
| Processing | of chromatographic data is | | | | |
| | performed and if reprocessed, | | | | |
| | whether there exist procedures | | | | |
| | in place to handle and document | | | | |
| | such reprocessing | | | | |
| Data | Checking whether a copy is | Essential | 0.40% | Yes | 0.40% |
| Processing | saved only of the final results | | | | |
| | from reprocessed laboratory | | | | |
| | chromatography and not the | | | | |
| | entire sequence | | | | |
| Data | Checking whether manual | Essential | 0.40% | Yes | 0.40% |
| Processing | integration of chromatograms is | | | | |
| | practiced and whether the | | | | |
| | software allows saving of | | | | |
| | manually integrated | | | | |
| | chromatograms or test results on | | | | |
| | desktops or temporary locations | | | | |
| Data | Checking whether there exist | Essential | 0.40% | Yes | 0.40% |
| Processing | controls to track or check if trial | | | | |
| | samples are analysed before the | | | | |
| | original sample | | | | |
| Data | Checking whether the site team | Essential | 0.40% | Yes | 0.40% |
| Transfer/migration | has a robust and validated data | | | | |
| | transfer or migration or retention | | | | |
| | procedure to ensure that data | | | | |
| | integrity is maintained during | | | | |
| | the data lifecycle | | | | |
| Data | Checking whether there is a data | Essential | 0.40% | Yes | 0.40% |
| Transfer/migration | migration audit log, whether | | | | |
| | there were challenge tests | | | | |
| | performed on the migrated data | | | | |
| | to ensure there is no alteration at | | | | |
| | each stage of data generation | | | | |
| Data | Checking whether the migration | Essential | 0.40% | Yes | 0.40% |
| Transfer/migration | failures (if any) are logged | | | | |
| | through QMS? | | | | |
| Data | Checking whether there exists | Essential | 0.40% | Yes | 0.40% |
| Governance | an SOP on Data Integrity which | | | | |
| | defines data governance at the | | | | |
| | site and whether the data | | | | |
| | ownership and accountability is | | | | |
| | defined in the SOP | | | | |
| Data | Checking whether the procedure | Essential | 0.40% | Yes | 0.40% |
| Governance | describes the method to handle | | | | |
| | data integrity incident observed | | | | |
| | (if any) at the site | | | | |
| Data | Checking whether periodic | Essential | 0.40% | Yes | 0.40% |
| Governance | audits are conducted for data | | | | |
| | integrity failures within the | | | | |
| | organisation's systems | | | | |
| Data | Checking whether the DI | Essential | 0.40% | Yes | 0.40% |
| Governance | behaviour aspects are a part of | | | | |
| | performance indicators | | | | |
| Data | Checking whether the site | Essential | 0.40% | Yes | 0.40% |
| Governance | performs a data governance | | | | |
| | review as part of their vendor | | | | |
| | assurance programme | | | | |
| DIRA | Checking whether DIRA is | Essential | 0.40% | Yes | 0.40% |
| | performed at the site | | | | |
| DIRA | Checking whether data integrity | Essential | 0.40% | Yes | 0.40% |
| | risk assessment (or equivalent) | | | | |
| | considers factors required to | | | | |
| | follow a process or perform a | | | | |
| | function including not only a | | | | |
| | computerised system but also | | | | |
| | the supporting people, guidance, | | | | |
| | training and quality systems | | | | |
| DIRA | Checking whether there are | Essential | 0.40% | Yes | 0.40% |
| | procedures in place to minimize | | | | |
| | the potential risk to data | | | | |
| | integrity including identifying | | | | |
| | the residual risk using risk | | | | |
| | management techniques | | | | |
| DIRA | Checking whether the site has a | Essential | 0.40% | Yes | 0.40% |
| | core team or SME who performs | | | | |
| | the DIRA | | | | |
| DIRA | Checking whether the DIRA | Essential | 0.40% | Yes | 0.40% |
| | report highlights areas for | | | | |
| | remediation including, | | | | |
| | 1) documentation of | | | | |
| | prioritisation of actions | | | | |
| | (including acceptance of an | | | | |
| | appropriate level of residual | | | | |
| | risk), | | | | |
| | 2) communication to | | | | |
| | management, subject to review, | | | | |
| | and | | | | |
| | 3) implementing risk-reducing | | | | |
| | short-term measures in | | | | |
| | situations where long-term | | | | |
| | remediation actions are | | | | |
| | identified to provide acceptable | | | | |
| | data governance in the interim | | | | |
| Computerised | Checking whether electronic | Essential | 0.40% | Yes | 0.40% |
| system | modules (document | | | | |
| transactions | management or Trackwise) used | | | | |
| | for QMS system are 21 CFR part | | | | |
| | 11/EU annex 11 compliant | | | | |
| Computerised | Checking whether the systems | Essential | 0.40% | Yes | 0.40% |
| system | used at site are capable of saving | | | | |
| transactions | data to permanent memory | | | | |
| | before prompting users to make | | | | |
| | changes | | | | |
| Computerised | Checking whether in case of | Essential | 0.40% | Yes | 0.40% |
| system | transactional systems, any | | | | |
| transactions | combination of multiple unit | | | | |
| | operations into a combined | | | | |
| | single transaction is avoided and | | | | |
| | whether the time interval before | | | | |
| | saving the data in computerized | | | | |
| | system is minimized | | | | |
| Computerised | Checking whether there exists a | Essential | 0.40% | Yes | 0.40% |
| system | process of changing time due to | | | | |
| transactions | day light savings and if it exists, | | | | |
| | whether there is a procedure and | | | | |
| | controls in place, whether this is | | | | |
| | done by authorized individual | | | | |
| | and whether this is documented | | | | |
| Computerised | Checking whether the date and | Essential | 0.40% | Yes | 0.40% |
| system | time change setting is disabled at | | | | |
| transactions | all computer workstations on | | | | |
| | site | | | | |
| Computerised | Checking whether time | Essential | 0.40% | Yes | 0.40% |
| system | management system is validated | | | | |
| transactions | to ensure correct logging of date | | | | |
| | and time of attendance | | | | |
| Computerised | Checking whether there exists a | Essential | 0.40% | Yes | 0.40% |
| system user | procedure to create and maintain | | | | |
| access/system | user account in electronic | | | | |
| administrator | system and whether the system | | | | |
| roles | has control to avoid duplicate | | | | |
| | user creation | | | | |
| Computerised | Checking whether there exists | Essential | 0.40% | Yes | 0.40% |
| system user | an approved user access matrix | | | | |
| access/system | for all the users and whether this | | | | |
| administrator | access matrix exactly | | | | |
| roles | corresponds with the actual user | | | | |
| | privileges while using the | | | | |
| | systems | | | | |
| Computerised | Checking whether the access | Essential | 0.40% | Yes | 0.40% |
| system user | controls are applied to both the | | | | |
| access/system | operating system and | | | | |
| administrator | application levels and whether | | | | |
| roles | the access privileges list are | | | | |
| | periodically updated | | | | |
| Computerised | Checking whether sufficient | Critical | 2.86% | Yes | 2.86% |
| system user | controls are available to prevent | | | | |
| access/system | unauthorized access to system or | | | | |
| administrator | to change or delete the data | | | | |
| roles | | | | | |
| Computerised | Checking whether there exist | Essential | 0.40% | Yes | 0.40% |
| system user | instruments or computer | | | | |
| access/system | systems that have shared logins, | | | | |
| administrator | whether generic user or system | | | | |
| roles | admin access is used for | | | | |
| | generating, amending or storing | | | | |
| | data for Good Practices (GxP) | | | | |
| | systems and whether there are | | | | |
| | appropriate procedures available | | | | |
| | to ensure user controls and data | | | | |
| | integrity | | | | |
| Computerised | Checking whether all users have | Essential | 0.40% | Yes | 0.40% |
| system user | individual login ID and | | | | |
| access/system | password to the respective | | | | |
| administrator | instrument or system where | | | | |
| roles | electronic data or record is | | | | |
| | generated or maintained and in | | | | |
| | absence thereof, whether | | | | |
| | appropriate controls are in place | | | | |
| | to ensure data integrity | | | | |
| Computerised | Checking whether the system | Essential | 0.40% | Yes | 0.40% |
| system User | administrator for all | | | | |
| access/System | computerised systems is | | | | |
| administrator | independent from the user and | | | | |
| roles | whether same person acting as | | | | |
| | user as well as administrator | | | | |
| Computerised | Checking whether there are | Essential | 0.40% | Yes | 0.40% |
| system User | multiple system administrators | | | | |
| access/System | for a single system and if there | | | | |
| administrator | are such multiple system | | | | |
| roles | administrators, whether they are | | | | |
| | restricted to minimum number | | | | |
| | of people and whether they use | | | | |
| | individual ID and password | | | | |
| Computerised | Checking whether only system | Essential | 0.40% | Yes | 0.40% |
| system User | administrator has rights to alter | | | | |
| access/System | files and settings and if they | | | | |
| administrator | have such rights, whether there | | | | |
| roles | exists record of all the actions | | | | |
| | generated | | | | |
| Computerised | Checking whether appropriate | Essential | 0.40% | Yes | 0.40% |
| system user | assessments and controls are | | | | |
| access/system | available for systems partially | | | | |
| administrator | used for GxP purposes, | | | | |
| roles | approved suppliers, stock status, | | | | |
| | location and transaction | | | | |
| | histories and similar purposes | | | | |
| Computerised | Checking whether if no suitable | Essential | 0.40% | Yes | 0.40% |
| system user | alternative computerised system | | | | |
| access/system | is available, whether equivalent | | | | |
| administrator | controls are provided by third- | | | | |
| roles | party software or a paper-based | | | | |
| | method of providing traceability | | | | |
| | (with version control) | | | | |
| Computerised | Checking whether there exists a | Essential | 0.40% | Yes | 0.40% |
| system | procedure detailing the | | | | |
| transactions | password complexity, number of | | | | |
| | wrong password attempts and | | | | |
| | password change frequency | | | | |
| | (system generated) | | | | |
| Computerised | Checking whether password | Essential | 0.40% | Yes | 0.40% |
| system | change prompt is activated | | | | |
| transactions | asking for the change on fixed | | | | |
| | periodicity | | | | |
| Computerised | Checking whether the computer | Essential | 0.40% | Yes | 0.40% |
| system | system gets timed out within | | | | |
| transactions | limited time if not worked on as | | | | |
| | per Information Technology | | | | |
| | (IT) policy requiring re-entering | | | | |
| | of password, and if the user | | | | |
| | leaves the workstation, whether | | | | |
| | procedures and/or automatic | | | | |
| | controls define and ensure that it | | | | |
| | is treated as a non-continuous | | | | |
| | session | | | | |
| Audit trail | Checking whether audit trail is | Essential | 0.40% | Yes | 0.40% |
| | activated for all your | | | | |
| | computerised systems | | | | |
| Audit trail | Checking whether Audit trail | Essential | 0.40% | Yes | 0.40% |
| | includes the following: | | | | |
| | User Name | | | | |
| | Date and Time | | | | |
| | Reason for change | | | | |
| | Integration parameter used in | | | | |
| | case of chromatograph | | | | |
| | Reprocessing details | | | | |
| | Change History | | | | |
| | Changes to sample run | | | | |
| | sequence (as applicable) | | | | |
| | Change in critical process | | | | |
| | parameters to achieve a more | | | | |
| | desirable result (as applicable) | | | | |
| | esignature | | | | |
| Audit trail | Checking whether user is able to | Critical | 2.86% | Yes | 2.86% |
| | amend (switch on/off) the audit | | | | |
| | trail settings and edit the raw | | | | |
| | data (including chromatographic | | | | |
| | data) for any computerized | | | | |
| | systems | | | | |
| Audit trail | Checking whether there exist | Essential | 0.40% | Yes | 0.40% |
| | optimal controls and measures to | | | | |
| | evaluate data generated by the | | | | |
| | message centre in the form of an | | | | |
| | audit trail related to alarms, | | | | |
| | warning, error or run time data | | | | |
| | and whether there is an SOP | | | | |
| | governing the same | | | | |
| Audit Trail | Checking whether the electronic | Essential | 0.40% | Yes | 0.40% |
| | record has system generated date | | | | |
| | and time stamp, and if the time | | | | |
| | on the system and report is | | | | |
| | synchronized | | | | |
| Audit trail | Checking whether there exists a | Essential | 0.40% | Yes | 0.40% |
| | procedure to periodically review | | | | |
| | the appropriate audit trails and | | | | |
| | data integrity review to verify | | | | |
| | adherence to written procedures | | | | |
| | (to verify privileges assigned to | | | | |
| | users, creation of project folders, | | | | |
| | data generated and maintained | | | | |
| | correctly) | | | | |
| Audit trail | Checking whether there exists a | Essential | 0.40% | Yes | 0.40% |
| | procedure in place that describes | | | | |
| | audit trail review requirements: | | | | |
| | frequency and documentation | | | | |
| | of its review, | | | | |
| | date of review, reviewed by and | | | | |
| | outcome of review, | | | | |
| | identification of actions required | | | | |
| | in case of non compliance, | | | | |
| | indicators of unauthorized | | | | |
| | changes | | | | |
| Audit trail | Checking whether there are | Critical | 2.86% | Yes | 2.86% |
| | enough controls to ensure that | | | | |
| | all original chromatographic | | | | |
| | data is restricted from editing | | | | |
| | once finalized or approved or | | | | |
| | locked | | | | |
| Audit trail | Checking whether the editing of | Critical | 2.86% | Yes | 2.86% |
| | point (j)8 is detectable in audit | | | | |
| | trails and whether such aspects | | | | |
| | are considered as part of review | | | | |
| | of audit trail | | | | |
| Electronic | Checking whether there exists | Essential | 0.40% | Yes | 0.40% |
| signatures | an SOP or policy on use or | | | | |
| | handling of e-signatures | | | | |
| Electronic | Checking whether there exists | Essential | 0.40% | Yes | 0.40% |
| signatures | an appropriate validation of | | | | |
| | signature process associated | | | | |
| | with the system | | | | |
| Electronic | Checking whether a record is | Essential | 0.40% | Yes | 0.40% |
| signatures | maintained of all the associates | | | | |
| | who apply their e-signature | | | | |
| | instead of their handwritten | | | | |
| | signatures | | | | |
| Electronic | Checking whether all the | Essential | 0.40% | Yes | 0.40% |
| signatures | computerised systems where | | | | |
| | electronic signatures are applied, | | | | |
| | document the specific person or | | | | |
| | title who signed the records | | | | |
| | electronically along with date | | | | |
| | and time stamp and the meaning | | | | |
| | of the signature (verified or | | | | |
| | approved) | | | | |
| Electronic | Checking whether there exists a | Essential | 0.40% | Yes | 0.40% |
| signatures | process of inserting image of the | | | | |
| | signature or a footnote | | | | |
| | indicating that the document has | | | | |
| | been electronically signed | | | | |
| | (where this has been entered by | | | | |
| | a means other than the validated | | | | |
| | electronic signature process) | | | | |
| Data review | Checking whether there exists a | Essential | 0.40% | Yes | 0.40% |
| and approval | procedure that describes the | | | | |
| | process for review and approval | | | | |
| | of data and whether the raw data | | | | |
| | is accessible with appropriate | | | | |
| | control to person performing | | | | |
| | data checking activities on | | | | |
| | system | | | | |
| Data review | Checking whether the data | Essential | 0.40% | Yes | 0.40% |
| and approval | review includes risk based | | | | |
| | review of relevant meta data | | | | |
| | including audit trails and | | | | |
| | whether the same is documented | | | | |
| Data review | Checking whether there exists a | Essential | 0.40% | Yes | 0.40% |
| and approval | practice of thorough data review | | | | |
| | for all applicable electronic | | | | |
| | source data to verify scientific | | | | |
| | integrity of the reported results, | | | | |
| | to verify adherence to | | | | |
| | procedures or test methods and | | | | |
| | to verify that all results are | | | | |
| | reported and accounted for; | | | | |
| | whether there is an SOP | | | | |
| | governing the same | | | | |
| Data review | Checking whether the | Essential | 0.40% | Yes | 0.40% |
| and approval | equipment raw data is | | | | |
| | periodically reviewed, | | | | |
| | reconciled against paper records | | | | |
| | and extracted as electronic data | | | | |
| | where the equipment does not | | | | |
| | store electronic data | | | | |
| | permanently and only holds a | | | | |
| | certain volume before | | | | |
| | overwriting | | | | |
| Data review | Checking whether there is any | Essential | 0.40% | Yes | 0.40% |
| and approval | procedure for periodic review or | | | | |
| | audit of electronic data | | | | |
| | generated, which verifies the | | | | |
| | effectiveness of existing control | | | | |
| | measures and the possibility of | | | | |
| | unauthorised activity | | | | |
| Data review | Checking whether there are | Essential | 0.40% | Yes | 0.40% |
| and approval | controls to verify the reported | | | | |
| | data and actual data | | | | |
| Data review | Checking whether customized | Essential | 0.40% | Yes | 0.40% |
| and approval | reports in computer systems are | | | | |
| | validated and locked to prevent | | | | |
| | changes | | | | |
| Data Retention | Checking whether there is a | Essential | 0.40% | Yes | 0.40% |
| | retention policy for of all records | | | | |
| | (manual & electronic), soft data | | | | |
| | and meta data, and whether it | | | | |
| | covers periodicity for back up | | | | |
| | and retention of both electronic | | | | |
| | and paper records along with | | | | |
| | meta data | | | | |
| Data retention | Checking whether there are | Critical | 2.86% | Yes | 2.86% |
| | procedures for destruction of | | | | |
| | data (blank forms, reports, | | | | |
| | training records, work sheets, | | | | |
| | raw data files, logbooks) which | | | | |
| | consider data criticality | | | | |
| Data Retention | Checking whether there are data | Essential | 0.40% | Yes | 0.40% |
| | and document retention | | | | |
| | arrangements available to ensure | | | | |
| | the protection of records from | | | | |
| | deliberate or inadvertent | | | | |
| | alteration or loss | | | | |
| Data Retention | Checking whether the data | Essential | 0.40% | Yes | 0.40% |
| | retention process of the sites | | | | |
| | include: | | | | |
| | verification and availability of | | | | |
| | copies of all raw data (lab note | | | | |
| | book, logs, balance printouts), | | | | |
| | metadata, | | | | |
| | check for availability of | | | | |
| | relevant audit trail and result | | | | |
| | files, | | | | |
| | any variable software or system | | | | |
| | configuration settings specific to | | | | |
| | each record, and all data | | | | |
| | processing runs (including | | | | |
| | methods and audit trails) | | | | |
| | necessary for reconstruction of a | | | | |
| | given raw data set | | | | |
| Data Retention | Checking whether metadata | Essential | 0.40% | Yes | 0.40% |
| | includes following components | | | | |
| | while retention: | | | | |
| | a date or time stamp for when | | | | |
| | the data were acquired, | | | | |
| | a user ID of the person who | | | | |
| | conducted the test or analysis | | | | |
| | that generated the data, | | | | |
| | instrument ID used to acquire | | | | |
| | the data, | | | | |
| | audit trails | | | | |
| Data Retention | Checking whether BMRs and | Essential | 0.40% | Yes | 0.40% |
| | BPRs are centrally archived | | | | |
| | under QA custody. whether | | | | |
| | access to this is controlled and | | | | |
| | restricted (manually controlled | | | | |
| | or electronically controlled | | | | |
| | through combination lock, | | | | |
| | biometrics or access card) | | | | |
| Data Retention | Checking whether there is any | Essential | 0.40% | Yes | 0.40% |
| | data generation through | | | | |
| | photograph, image or any other | | | | |
| | media and if there is such data | | | | |
| | generation, whether there are | | | | |
| | procedures and controls to | | | | |
| | maintain their storage | | | | |
| | throughout the lifecycle | | | | |
| Data Retention | Checking whether in case, | Essential | 0.40% | Yes | 0.40% |
| | where the original format cannot | | | | |
| | be retained due to degradation | | | | |
| | issues, whether there are | | | | |
| | alternative mechanisms for | | | | |
| | recording (photography or | | | | |
| | digitisation) and subsequent | | | | |
| | storage considered and selection | | | | |
| | rationale documented | | | | |
| Data Retention | Checking whether the data (or a | Essential | 0.40% | Yes | 0.40% |
| | true copy) generated in paper | | | | |
| | format is retained by using a | | | | |
| | validated scanning process, and | | | | |
| | if so retained, whether there is a | | | | |
| | documented process in place to | | | | |
| | ensure that the outcome is a true | | | | |
| | copy | | | | |
| Data Retention | Checking whether there are | Essential | 0.40% | Yes | 0.40% |
| | adequate controls to prevent data | | | | |
| | tamper or deletion and data loss | | | | |
| | within the software or outside | | | | |
| | the software application | | | | |
| Data Retention | Checking whether the soft data | Essential | 0.40% | Yes | 0.40% |
| | can be located, retrieved, | | | | |
| | presented and interpreted in its | | | | |
| | original or acceptable form | | | | |
| Backup & | Checking whether validated | Essential | 0.40% | Yes | 0.40% |
| Archive | procedures are available for data | | | | |
| | backup and archive, and if | | | | |
| | available, whether it includes, | | | | |
| | i. periodic integrity check, | | | | |
| | ii. accuracy of backup data, | | | | |
| | readability of data or metadata | | | | |
| | and | | | | |
| | the ability to restore the data as | | | | |
| | per its original validated state | | | | |
| | including legacy systems to | | | | |
| | confirm the continued support of | | | | |
| | legacy computerised systems | | | | |
| Backup & | Checking whether a procedure is | Essential | 0.40% | Yes | 0.40% |
| Archive | in place for archival of records | | | | |
| | prior to decommissioning the | | | | |
| | computerised system | | | | |
| Backup & | Checking whether data security | Essential | 0.40% | Yes | 0.40% |
| Archive | controls exist on manufacturing | | | | |
| | software or Programmable | | | | |
| | Logic Controllers (PLCs), and | | | | |
| | whether it is backed up | | | | |
| | periodically to prevent data loss | | | | |
| | in case of failure | | | | |
| Backup & | Checking where hybrid records | Essential | 0.40% | Yes | 0.40% |
| Archive | are stored and whether | | | | |
| | references between physical and | | | | |
| | electronic records are | | | | |
| | maintained such that full | | | | |
| | verification of events is possible | | | | |
| | throughout the retention period | | | | |
| Backup & | Checking whether the archival | Essential | 0.40% | Yes | 0.40% |
| Archive | area is suitably controlled and | | | | |
| | whether it is inspected at regular | | | | |
| | intervals | | | | |
| Backup & | Checking whether the site is | Essential | 0.40% | Yes | 0.40% |
| Archive | using any third party service | | | | |
| | provider for archival of data and | | | | |
| | if used, whether there is a | | | | |
| | contract in place which defines | | | | |
| | the responsibilities for archiving | | | | |
| | and continued readability of the | | | | |
| | data throughout the retention | | | | |
| | period | | | | |
| Backup & | Checking whether the backup | Essential | 0.40% | Yes | 0.40% |
| Archive | strategies for the data owners are | | | | |
| | documented | | | | |
| Backup & | Checking whether adequate | Essential | 0.40% | Yes | 0.40% |
| Archive | back up control, disaster | | | | |
| | management and server | | | | |
| | redundancy for chromatographic | | | | |
| | data exists | | | | |
| Backup & | Checking whether periodic | Critical | 2.86% | Yes | 2.86% |
| Archive | review or retrieval of | | | | |
| | chromatographic records is | | | | |
| | performed, if performed whether | | | | |
| | it is performed with limited | | | | |
| | access control and whether this | | | | |
| | is documented | | | | |
| Backup & | Checking whether disaster | Essential | 0.40% | Yes | 0.40% |
| Archive | management exists for archival | | | | |
| | area where BMR or BPR are | | | | |
| | archived | | | | |
| File structure | Checking whether there exists a | Essential | 0.40% | Yes | 0.40% |
| | process for file structure and | | | | |
| | naming conventions within the | | | | |
| | GxP environment for | | | | |
| | management of electronic data | | | | |
| | to facilitate effective data review | | | | |
| | and ensure that all results are | | | | |
| | reported and accounted for; | | | | |
| | including naming of folders or | | | | |
| | files, sequences and samples as | | | | |
| | applicable | | | | |
| File structure | Checking whether there exists a | Essential | 0.40% | Yes | 0.40% |
| | written procedure to control data | | | | |
| | acquisition in appropriate | | | | |
| | project folders and whether there | | | | |
| | exist appropriate access controls | | | | |
| | on these folders | | | | |
| Validation | Checking whether computerized | Essential | 0.40% | Yes | 0.40% |
| | systems are validated as per | | | | |
| | regulatory requirements for their | | | | |
| | intended purpose | | | | |
| Validation | Checking whether the extent of | Essential | 0.40% | Yes | 0.40% |
| | validation is based on justified | | | | |
| | and documented risk assessment | | | | |
| Validation | Checking whether all PLCs | Essential | 0.40% | Yes | 0.40% |
| | containing recipes or work | | | | |
| | instructions are validated as per | | | | |
| | Good Automated Manufacturing | | | | |
| | Practice (GAMP) 5 or for 21 | | | | |
| | CFR part 11 controls | | | | |
| Validation | Checking whether excel work | Essential | 0.40% | Yes | 0.40% |
| | sheets if used in Quality Control | | | | |
| | (QC) or any other function are | | | | |
| | validated and whether these cells | | | | |
| | are locked, access is controlled | | | | |
| | and saving or copying of these | | | | |
| | are disabled | | | | |
| Validation | Checking whether validation for | Essential | 0.40% | Yes | 0.40% |
| | intended purpose ensures that | | | | |
| | the steps for generating the | | | | |
| | custom report accurately reflect | | | | |
| | those described in the data | | | | |
| | checking SOP? | | | | |
| Validation | Checking whether each | Essential | 0.40% | Yes | 0.40% |
| | workflow on all the computer | | | | |
| | systems or softwares in use are | | | | |
| | validated | | | | |
| Validation | Checking whether the critical | Essential | 0.40% | Yes | 0.40% |
| | steps are defined based on the | | | | |
| | functionality of system at User | | | | |
| | Requirement Specification stage | | | | |
| | of a software | | | | |
| Validation | Checking whether computer | Essential | 0.40% | Yes | 0.40% |
| | systems designed to ensure that | | | | |
| | the execution of critical steps are | | | | |
| | recorded contemporaneously | | | | |
| Validation | Checking whether the functional | Essential | 0.40% | Yes | 0.40% |
| | verification demonstrates that | | | | |
| | the required information is | | | | |
| | consistently and completely | | | | |
| | presented | | | | |
| Validation | Checking whether periodic | Essential | 0.40% | Yes | 0.40% |
| | review of validation status of | | | | |
| | systems is performed and | | | | |
| | documented | | | | |
| Validation | Checking whether there exists a | Essential | 0.40% | Yes | 0.40% |
| | process of retirement of | | | | |
| | computerized systems governed | | | | |
| | by an SOP | | | | |
| IT Suppliers | Checking whether there exists | Essential | 0.40% | Yes | 0.40% |
| and Service | an inventory of all applicable | | | | |
| Providers | computerised systems both live | | | | |
| | and retired and whether they are | | | | |
| | periodically reviewed | | | | |
| IT Suppliers | Checking whether the site uses | Essential | 0.40% | Yes | 0.40% |
| and Service | any ‘cloud’ or ‘virtual’ services | | | | |
| Providers | from third party, and if used, | | | | |
| | whether there is an | | | | |
| | understanding of the services | | | | |
| | provided, ownership, retrieval, | | | | |
| | retention and security of the data | | | | |
| IT Suppliers | Checking whether the contract | Essential | 0.40% | Yes | 0.40% |
| and Service | or technical agreements of third | | | | |
| Providers | party vendor include | | | | |
| | responsibilities of contract giver | | | | |
| | and acceptor, clause on data | | | | |
| | ownership, governance and | | | | |
| | accessibility | | | | |
| IT Suppliers | Checking whether in case of | Essential | 0.40% | Yes | 0.40% |
| and Service | cloud services, the geographical | | | | |
| Providers | location laws are considered | | | | |
| | where the data is physically | | | | |
| | located | | | | |
| IT Suppliers | Checking whether there exists a | Essential | 0.40% | Yes | 0.40% |
| and Service | process of auditing the cloud and | | | | |
| Providers | software service provider | | | | |
| IT Suppliers | Checking whether there is an | Essential | 0.40% | Yes | 0.40% |
| and Service | external vendor used for | | | | |
| Providers | generating summary reports for | | | | |
| | the site or organization, and if | | | | |
| | such vendor exists whether the | | | | |
| | vendor is evaluated for the data | | | | |
| | integrity controls and processes | | | | |
| | prior to using the information | | | | |
| IT Suppliers | Checking whether consultants | Essential | 0.40% | Yes | 0.40% |
| and Service | are appointed at the site, and if | | | | |
| Providers | appointed, whether there exist | | | | |
| | procedural controls to prevent | | | | |
| | access to company data through | | | | |
| | secured network | | | | |
| IT Suppliers | Checking whether there exists | Essential | 0.40% | Yes | 0.40% |
| and Service | business continuity for all the | | | | |
| Providers | critical softwares used at site | | | | |
| IT Suppliers | Checking whether the business | Essential | 0.40% | Yes | 0.40% |
| and Service | continuity arrangement (manual | | | | |
| Providers | or alternative system) is | | | | |
| | documented in contract and | | | | |
| | tested, and whether the audit of | | | | |
| | the service provider is | | | | |
| | performed | | | | |
| QMS | Checking whether changes to | Essential | 0.40% | Yes | 0.40% |
| | qualified electronic system are | | | | |
| | routed through change | | | | |
| | management and risk | | | | |
| | assessment | | | | |
| QMS | Checking whether all deviations | Essential | 0.40% | Yes | 0.40% |
| | or incidences immediately upon | | | | |
| | their discovery, are documented | | | | |
| QMS | Checking whether the deviation | Essential | 0.40% | Yes | 0.40% |
| | investigations (not only failures | | | | |
| | and data errors) include | | | | |
| | determining and correcting the | | | | |
| | root cause of the deviation and a | | | | |
| | thorough impact assessment | | | | |
| QMS | Checking whether the | Essential | 0.40% | Yes | 0.40% |
| | Information Technology related | | | | |
| | incident or deviations within | | | | |
| | QMS are logged and tracked | | | | |
| QMS | Checking whether the OOS | Essential | 0.40% | Yes | 0.40% |
| | handling procedure mentions | | | | |
| | when retest is permitted and | | | | |
| | when approval to do so is | | | | |
| | required | | | | |
| QMS | Checking whether there exists a | Essential | 0.40% | Yes | 0.40% |
| | practice of frequent invalidation | | | | |
| | of OOS, in case of invalidated | | | | |
| | OOS, whether a thorough | | | | |
| | investigation is undertaken for | | | | |
| | all, and whether there are | | | | |
| | repeated errors for invalidating | | | | |
| | the OOS | | | | |
| QMS | Checking whether there exists | Essential | 0.40% | Yes | 0.40% |
| | an SOP for repeat testing which | | | | |
| | includes scientific justification | | | | |
| | and documentation requirements | | | | |
| | for the same | | | | |
| QMS | Checking whether there exists a | Essential | 0.40% | Yes | 0.40% |
| | procedure in place to handle, if | | | | |
| | any abnormality or incident | | | | |
| | related to software is observed | | | | |
| | during operation | | | | |
| Calibration | Checking whether the simple | Essential | 0.40% | Yes | 0.40% |
| | electronic systems (pH meters, | | | | |
| | balances, thermometers and | | | | |
| | similar electronic systems) are | | | | |
| | calibrated | | | | |
| Quality Control | Checking whether the site has a | Essential | 0.40% | Yes | 0.40% |
| | practice of trial injection in | | | | |
| | chromatographic analysis and | | | | |
| | deletion of sequences | | | | |
| Quality Control | Checking whether there exists a | Essential | 0.40% | Yes | 0.40% |
| | practice of using actual test | | | | |
| | samples for system suitability or | | | | |
| | whether any test or equilibration | | | | |
| | runs are carried out | | | | |
| Standalone | Checking whether all QC | Critical | 2.86% | Yes | 2.86% |
| systems | chromatographic systems or | | | | |
| | standalone instruments | | | | |
| | (balances, moisture analyser, | | | | |
| | ultra violet (UV) | | | | |
| | spectrophotometer, pH meter) | | | | |
| | are 21 CFR Part 11/EU Annex | | | | |
| | 11 compliant, and whether there | | | | |
| | exist entry level controls in these | | | | |
| | computers followed by system | | | | |
| | level access controls | | | | |
| Standalone | Checking whether there exist | Critical | 2.86% | Yes | 2.86% |
| systems | any standalone instruments | | | | |
| | (balances, moisture meter, UV | | | | |
| | spectrophotometer, pH meter) | | | | |
| | which are not attached with | | | | |
| | printers, and if they exist, | | | | |
| | whether they are validated and | | | | |
| | with adequate controls for | | | | |
| | capturing, documenting and | | | | |
| | second level review of the data | | | | |
| | and audit trails | | | | |
| Standalone | Checking whether in case of non | Critical | 2.86% | Yes | 2.86% |
| systems | availability of audit trail | | | | |
| | functionality (standalone | | | | |
| | instruments) and individual user | | | | |
| | account expectations, whether | | | | |
| | there exists an alternate robust | | | | |
| | control procedure for data | | | | |
| | review and retention along with | | | | |
| | justification, including legacy | | | | |
| | systems | | | | |
| Standalone | Checking whether there exist | Essential | 0.40% | Yes | 0.40% |
| systems | plans to upgrade standalone | | | | |
| | systems, and whether the | | | | |
| | required budget is proposed | | | | |
| Standalone | Checking whether computers | Essential | 0.40% | Yes | 0.40% |
| systems | are connected to | | | | |
| | chromatographic system and/or | | | | |
| | Supervisory Control and Data | | | | |
| | Acquisition (SCADA) are | | | | |
| | disabled for download of any | | | | |
| | other application (excel, power | | | | |
| | point, word), whether it is used | | | | |
| | exclusively for the data | | | | |
| | acquisition system, and whether | | | | |
| | it is confirmed that it does not | | | | |
| | allow download or saving of any | | | | |
| | data and subsequent changes to | | | | |
| | it | | | | |
| Standalone | Checking whether there exists a | Essential | 0.40% | Yes | 0.40% |
| systems | back-up of standalone | | | | |
| | instruments and whether the | | | | |
| | mechanism is validated | | | | |
| Trainings | Checking whether all personnel | Essential | 0.40% | Yes | 0.40% |
| | accessing the system or involved | | | | |
| | with the system have adequate | | | | |
| | training and whether their | | | | |
| | training records are available, | | | | |
| | whether their responsibilities are | | | | |
| | defined and documented to carry | | | | |
| | out their assigned duties | | | | |
| Trainings | Checking whether Data Integrity | Essential | 0.40% | Yes | 0.40% |
| | training is performed as part of | | | | |
| | induction for new joinees | | | | |
| Trainings | Checking whether periodic | Essential | 0.40% | Yes | 0.40% |
| | trainings to all GxP relevant | | | | |
| | support functions on importance | | | | |
| | of Data Integrity principle is | | | | |
| | provided as part of a routine | | | | |
| | annual training program | | | | |
| Trainings | Checking whether the personnel | Essential | 0.40% | Yes | 0.40% |
| | who review and conduct data | | | | |
| | integrity checks are trained and | | | | |
| | qualified on data integrity | | | | |
| | requirements | | | | |
| Trainings | Checking whether the senior | Essential | 0.40% | Yes | 0.40% |
| | management team is trained on | | | | |
| | data integrity requirements | | | | |
| Trainings | Checking whether there exists | Essential | 0.40% | Yes | 0.40% |
| | an SOP, policies, trainings or | | | | |
| | awareness sessions at site that | | | | |
| | helps prevent sharing of | | | | |
| | passwords or IDs | | | | |
| Manufacturing | Checking whether there exist | Essential | 0.40% | Yes | 0.40% |
| | PLC controls or automation to | | | | |
| | all key manufacturing | | | | |
| | equipment, such that defective | | | | |
| | product can be rejected based on | | | | |
| | defined tolerances | | | | |
| Manufacturing | Checking whether the processes | Essential | 0.40% | Yes | 0.40% |
| | and their validation are robust | | | | |
| | enough and challenged | | | | |
| | adequately to not produce a non- | | | | |
| | compliant product | | | | |
| Manufacturing | Checking whether there exists a | Essential | 0.40% | Yes | 0.40% |
| | qualified SCADA (where | | | | |
| | available) system and whether | | | | |
| | SCADA print is attached in | | | | |
| | BMR to prevent manual | | | | |
| | recording | | | | |
| Manufacturing | Do you have a SOP to assess and | Essential | 0.40% | Yes | 0.40% |
| | document Programmable Logic | | | | |
| | Controller (PLC) alarms based | | | | |
| | on its criticality ( nature, | | | | |
| | recording the duration and time) | | | | |
| | from an operational standpoint? | | | | |
| | If yes, is this also reviewed by | | | | |
| | the quality unit? | | | | |
| Manufacturing | Checking whether the PLC | Essential | 0.40% | Yes | 0.40% |
| | controls, alarms the user not | | | | |
| | only when the product is out of | | | | |
| | specification but when process | | | | |
| | steps out of the validated range | | | | |
| Manufacturing | Checking whether there exist | Essential | 0.40% | Yes | 0.40% |
| | digital clocks to record timing in | | | | |
| | the raw data or batch record, and | | | | |
| | if they exist, whether they are | | | | |
| | centrally controlled or | | | | |
| | synchronized for recording | | | | |
| | timed events specifying the time | | | | |
| | zone | | | | |
| Manufacturing | Checking whether for Active | Essential | 0.40% | No | 0.00% |
| | Pharmaceutical Ingredient (API) | | | | |
| | sites, whether critical parameters | | | | |
| | (temperature, pressure and pH) | | | | |
| | of reactors are automated, | | | | |
| | whether it prevents manual | | | | |
| | recording and whether print out | | | | |
| | from PLCs for these parameters | | | | |
| | are attached in BMR | | | | |
| Manufacturing | Checking whether equipment | Essential | 0.40% | Yes | 0.40% |
| | cleaning, sanitization and | | | | |
| | maintenance records | | | | |
| | are | | | | |
| | available and traceable at site | | | | |
| Manufacturing | Checking whether procedures | Essential | 0.40% | Yes | 0.40% |
| | are in place to monitor the output | | | | |
| | of the manufacturing processes | | | | |
| | that may be responsible for | | | | |
| | causing variability in the | | | | |
| | characteristics of in-process | | | | |
| | material and the drug product | | | | |
| Manufacturing | Checking whether the | Critical | 2.86% | Yes | 2.86% |
| | documents generated or printed | | | | |
| | from electronic systems in | | | | |
| | shopfloor are compliant to 21 | | | | |
| | CFR part 11 | | | | |

| | |
|---|---|
| Total DI Score | 95.93% |

Automation Score

| | | |
|---|---|---|
| QC | HPLC (1) | 1.0% |
| QC | Other QC instrument and | 1.5% |
| | webosphere (1.5) | |
| QC | LIMS (2.5) | 0.5% |
| | 0.5 if stability module in place | |
| QMS | Document Management (DM) - | 1.5% |
| | 1.5 | |
| QMS | Training Management (TM) - | 1.5% |
| | 1.5 | |
| QMS | Trackwise or any other | 2.0% |
| | depending on modules: | |
| | Deviation, CAPA, change, | |
| | complaint - 0.5 each total 2 | |
| Mfg | Manual: (0) | 2.5% |
| | Basic PLC/SCADA: (1) | |
| | Partial: (2.5) | |
| | Complete: (5) | |
| | Automation Score | 10.50% |
| | DI Score (85% ) of Total score | 81.54% |
| | 95.93% | |
| | Automation Score (15%) | 10.50% |
| | Site CALCULUS ™ Score | 92.04% |

Conclusion: The site's CALCULUS™ Score of 92.04% indicates that the site is highly data integrity compliant with ‘A+’ rating and requires regular monitoring for sustenance of the compliance.

### Example 2: Determination of PREDICT™ Rating

PREDICT™ rating improvement for Site #3:

The initial and revised (improved) System Score for Site #3 were determined before and after following actions/interventions were adopted resulting in an improvement in the PREDICT™ rating:
Revised and improved the cleaning procedure for inventory in storage area.
Updated the understanding on the QMS handling procedure to include the clarity in instruction for historical reviews and trending.
Update the monitoring frequency on Differential pressure in the classified areas.
Updated the tracking of the extensions of QMS actions.

The initial and revised (improved) System Score for Site #3 is shown in TABLE 8 as below:

TABLE 8

| | | | | | | Score | |
|---|---|---|---|---|---|---|---|
| | | | | Score (1- | | (1-4 | |
| | | | | 4 with 4 | | with 4 | |
| | | | | being | Weighted | being | Weighted |
| | | | | Max) | Score | Max) | Score |
| Category | Level | Parameter | Weightage | INITIAL SCORE | | REVISED SCORE | |
| Facility And | I | Equipment | 22% | 0 | 0.00 | 4 | 0.88 |
| Equipment | | cleaning & | | | | | |
| System | | sanitisation | | | | | |
| Facility And | I | Prevention of | 23% | 0 | 0.00 | 4 | 0.92 |
| Equipment | | cross- | | | | | |
| System | | contamination, | | | | | |
| | | isolation and | | | | | |
| | | containment | | | | | |
| Facility And | II | Air handling | 5% | 3 | 0.15 | 4 | 0.20 |
| Equipment | | systems | | | | | |
| System | | | | | | | |
| Facility And | II | Appropriate | 5% | 4 | 0.20 | 3 | 0.15 |
| Equipment | | use of equipment | | | | | |
| System | | operations | | | | | |
| | | substances, | | | | | |
| | | (lubricants, | | | | | |
| | | coolants, | | | | | |
| | | refrigerants, etc.) | | | | | |
| | | contacting | | | | | |
| | | products/containers/ | | | | | |
| | | etc | | | | | |
| Facility And | II | Calibration | 5% | 4 | 0.20 | 4 | 0.20 |
| Equipment | | program | | | | | |
| System | | | | | | | |
| Facility And | II | Clean rooms | 5% | 3 | 0.15 | 4 | 0.20 |
| Equipment | | control, | | | | | |
| System | | maintenance, | | | | | |
| | | & cleaning | | | | | |
| Facility And | II | Environmental | 5% | 2 | 0.10 | 4 | 0.20 |
| Equipment | | zoning | | | | | |
| System | | | | | | | |
| Facility And | II | Equipment | 5% | 4 | 0.20 | 2 | 0.10 |
| Equipment | | surfaces should | | | | | |
| System | | not be reactive, | | | | | |
| | | additive, or | | | | | |
| | | absorptive | | | | | |
| Facility And | II | Facilities | 5% | 2 | 0.10 | 4 | 0.20 |
| Equipment | | Maintenance | | | | | |
| System | | | | | | | |
| Facility And | II | IT GMP, | 5% | 4 | 0.20 | 4 | 0.20 |
| Equipment | | validation and | | | | | |
| System | | security | | | | | |
| Facility And | III | Adequacy of | 1% | 4 | 0.04 | 3 | 0.03 |
| Equipment | | equipment design, | | | | | |
| System | | size, and location | | | | | |
| Facility And | III | Control | 1% | 4 | 0.04 | 4 | 0.04 |
| Equipment | | system for | | | | | |
| System | | implementing | | | | | |
| | | changes in the | | | | | |
| | | equipment | | | | | |
| Facility And | III | Design | 1% | 4 | 0.04 | 4 | 0.04 |
| Equipment | | guidelines | | | | | |
| System | | | | | | | |
| Facility And | III | Documented | 1% | 4 | 0.04 | 4 | 0.04 |
| Equipment | | investigation into | | | | | |
| System | | any unexpected | | | | | |
| | | discrepancy | | | | | |
| Facility And | III | Equipment | 1% | 4 | 0.04 | 4 | 0.04 |
| Equipment | | identification | | | | | |
| System | | practices | | | | | |
| Facility And | III | Facilities | 1% | 2 | 0.02 | 4 | 0.04 |
| Equipment | | Cleaning | | | | | |
| System | | | | | | | |
| Facility And | III | IQ/OQ/PQ of | 1% | 2 | 0.02 | 4 | 0.04 |
| Equipment | | facilities, utilities | | | | | |
| System | | and | | | | | |
| | | equipment | | | | | |
| Facility And | III | Layout & | 1% | 4 | 0.04 | 4 | 0.04 |
| Equipment | | drawings | | | | | |
| System | | | | | | | |
| Facility And | III | Lighting, | 1% | 2 | 0.02 | 3 | 0.03 |
| Equipment | | potable water, | | | | | |
| System | | washing and toilet | | | | | |
| | | facilities, sewage | | | | | |
| | | and refuse | | | | | |
| | | disposal | | | | | |
| Facility And | III | Pest control | 1% | 4 | 0.04 | 4 | 0.04 |
| Equipment | | | | | | | |
| System | | | | | | | |
| Facility And | III | Planned | 1% | 2 | 0.02 | 4 | 0.04 |
| Equipment | | preventive | | | | | |
| System | | maintenance | | | | | |
| | | program | | | | | |
| Facility And | III | Sanitation of | 1% | 4 | 0.04 | 4 | 0.04 |
| Equipment | | the building | | | | | |
| System | | | | | | | |
| Facility And | III | Technical / | 1% | 4 | 0.04 | 3 | 0.03 |
| Equipment | | Design files | | | | | |
| System | | | | | | | |
| Facility And | III | Zone | 1% | 2 | 0.02 | 4 | 0.04 |
| Equipment | | Ownership | | | | | |
| System | | | | | | | |
| Laboratory | I | OOS & OOT | 7% | 4 | 0.29 | 4 | 0.29 |
| Control System | | Investigations | | | | | |
| Laboratory | I | Qualification | 7% | 4 | 0.29 | 4 | 0.29 |
| Control System | | & Validation of | | | | | |
| | | all QC methods | | | | | |
| Laboratory | I | Qualification | 7% | 3 | 0.21 | 4 | 0.29 |
| Control System | | of all Laboratory | | | | | |
| | | Equipment | | | | | |
| Laboratory | I | Raw data | 7% | 4 | 0.29 | 4 | 0.29 |
| Control System | | definition, | | | | | |
| | | standards, control | | | | | |
| | | & verification | | | | | |
| Laboratory | I | Reference | 7% | 3 | 0.2 | 4 | 0.29 |
| Control System | | standards | | | | | |
| | | management | | | | | |
| Laboratory | I | Specifications, | 7% | 4 | 0.29 | 4 | 0.29 |
| Control System | | standards, and | | | | | |
| | | sampling plans | | | | | |
| Laboratory | I | Stability | 7% | 4 | 0.29 | 3 | 0.21 |
| Control System | | testing | | | | | |
| Laboratory | II | Acceptance | 5% | 2 | 0.10 | 4 | 0.20 |
| Control System | | Activities and in- | | | | | |
| | | process release | | | | | |
| Laboratory | II | Adequacy of | 5% | 4 | 0.20 | 3 | 0.15 |
| Control System | | equipment and | | | | | |
| | | facility for | | | | | |
| | | intended use | | | | | |
| Laboratory | II | Calibration | 5% | 4 | 0.20 | 4 | 0.20 |
| Control System | | program | | | | | |
| Laboratory | II | Laboratory | 5% | 4 | 0.20 | 2 | 0.10 |
| Control System | | methods, | | | | | |
| | | standards | | | | | |
| | | and controls | | | | | |
| Laboratory | II | Retained | 5% | 4 | 0.20 | 4 | 0.20 |
| Control System | | samples, | | | | | |
| | | requirements and | | | | | |
| | | management | | | | | |
| Laboratory | II | Sample | 5% | 4 | 0.20 | 4 | 0.20 |
| Control System | | handling, storage, | | | | | |
| | | and integrity | | | | | |
| Laboratory | II | Validation & | 5% | 2 | 0.10 | 4 | 0.20 |
| Control System | | security of | | | | | |
| | | computerized or | | | | | |
| | | automated | | | | | |
| | | processes | | | | | |
| Laboratory | III | Adequacy of | 3% | 4 | 0.10 | 4 | 0.10 |
| Control System | | staffing for | | | | | |
| | | laboratory | | | | | |
| | | operations | | | | | |
| Laboratory | III | Equipment | 3% | 4 | 0.10 | 4 | 0.10 |
| Control System | | List | | | | | |
| Laboratory | III | Maintenance | 3% | 4 | 0.10 | 4 | 0.10 |
| Control System | | program | | | | | |
| Laboratory | III | Method | 3% | 4 | 0.10 | 4 | 0.10 |
| Control System | | validation policy | | | | | |
| | | and standards | | | | | |
| Laboratory | III | Test data | 3% | 4 | 0.10 | 4 | 0.10 |
| Control System | | review and | | | | | |
| | | authorization | | | | | |
| Laboratory | III | Trending, | 3% | 4 | 0.10 | 2 | 0.05 |
| Control System | | reporting, and | | | | | |
| | | statistical quality | | | | | |
| | | control | | | | | |
| Material System | I | Environmentally | 8% | 4 | 0.33 | 4 | 0.33 |
| | | controlled | | | | | |
| | | storage conditions | | | | | |
| Material System | I | Finished | 8% | 3 | 0.25 | 2 | 0.17 |
| | | product | | | | | |
| | | distribution | | | | | |
| | | records by lot | | | | | |
| Material System | I | Identification | 8% | 4 | 0.33 | 4 | 0.33 |
| | | & Quality Status | | | | | |
| Material System | I | Lot | 8% | 4 | 0.33 | 4 | 0.33 |
| | | traceability of | | | | | |
| | | components | | | | | |
| | | making up a batch | | | | | |
| Material System | I | Purified | 8% | 3 | 0.25 | 4 | 0.33 |
| | | Water system | | | | | |
| | | control | | | | | |
| Material System | I | Specifications | 8% | 4 | 0.33 | 4 | 0.33 |
| | | and acceptance | | | | | |
| | | testing of raw | | | | | |
| | | materials | | | | | |
| Material System | II | API, | 3% | 4 | 0.12 | 4 | 0.12 |
| | | excipient, reagent | | | | | |
| | | and reference | | | | | |
| | | material control | | | | | |
| Material System | II | Bill of | 3% | 4 | 0.12 | 4 | 0.12 |
| | | Materials | | | | | |
| Material System | II | Control of | 3% | 4 | 0.12 | 4 | 0.12 |
| | | quarantine goods | | | | | |
| Material System | II | Control of | 3% | 3 | 0.09 | 3 | 0.09 |
| | | reject goods | | | | | |
| Material System | II | Expiry | 3% | 4 | 0.12 | 4 | 0.12 |
| | | dating and retest | | | | | |
| | | requirements | | | | | |
| Material System | II | Incoming | 3% | 3 | 0.09 | 4 | 0.12 |
| | | goods acceptance | | | | | |
| | | checks | | | | | |
| Material System | II | Lot | 3% | 4 | 0.12 | 4 | 0.12 |
| | | numbering control | | | | | |
| Material System | II | Pack Range | 3% | 4 | 0.12 | 4 | 0.12 |
| | | Control - | | | | | |
| | | Artwork, | | | | | |
| | | components, pack | | | | | |
| | | codes | | | | | |
| Material System | II | Qualification | 3% | 4 | 0.12 | 3 | 0.09 |
| | | of cold chain or | | | | | |
| | | supply chain | | | | | |
| Material System | II | Reconciliation | 3% | 4 | 0.12 | 4 | 0.12 |
| Material System | II | Reprocessing/ | 3% | 4 | 0.12 | 3 | 0.09 |
| | | rework control | | | | | |
| Material System | II | Sampling | 3% | 4 | 0.12 | 4 | 0.12 |
| | | plan | | | | | |
| Material System | III | Authority to | 2% | 4 | 0.08 | 4 | 0.08 |
| | | Destroy & | | | | | |
| | | destruction of | | | | | |
| | | records | | | | | |
| Material System | III | Control of | 2% | 4 | 0.08 | 3 | 0.06 |
| | | Distributors, | | | | | |
| | | records, Technical | | | | | |
| | | Agreements | | | | | |
| Material System | III | Control of | 2% | 4 | 0.08 | 4 | 0.08 |
| | | returned or | | | | | |
| | | salvaged goods | | | | | |
| Material System | III | Inventory | 2% | 4 | 0.08 | 4 | 0.08 |
| | | Management | | | | | |
| Material System | III | Raw | 2% | 4 | 0.08 | 4 | 0.08 |
| | | materials | | | | | |
| | | segregation and | | | | | |
| | | labelling | | | | | |
| Material System | III | Supplier | 2% | 2 | 0.04 | 4 | 0.08 |
| | | management | | | | | |
| Material System | III | Testing or | 2% | 4 | 0.08 | 4 | 0.08 |
| | | validation of | | | | | |
| | | supplier's test | | | | | |
| | | results for | | | | | |
| | | components, | | | | | |
| | | containers and | | | | | |
| | | closures | | | | | |
| Material System | III | Warehouse | 2% | 4 | 0.08 | 4 | 0.08 |
| | | controls and FIFO | | | | | |
| Packaging and | I | Adequate | 10% | 2 | 0.20 | 4 | 0.40 |
| Labelling System | | inspection | | | | | |
| | | (proofing) of | | | | | |
| | | incoming labeling | | | | | |
| Packaging and | I | Control of | 10% | 4 | 0.40 | 4 | 0.40 |
| Labelling System | | bulk & unlabelled | | | | | |
| | | product | | | | | |
| Packaging and | I | Examination | 10% | 4 | 0.40 | 4 | 0.40 |
| Labelling System | | of the labeled | | | | | |
| | | finished product | | | | | |
| Packaging and | I | In-process | 10% | 3 | 0.30 | 4 | 0.40 |
| Labelling System | | inspection of | | | | | |
| | | product | | | | | |
| Packaging and | I | Master | 10% | 3 | 0.30 | 4 | 0.40 |
| Labelling System | | Packaging | | | | | |
| | | Instructions and | | | | | |
| | | Records | | | | | |
| Packaging and | II | Conformance | 6% | 4 | 0.23 | 4 | 0.23 |
| Labelling System | | to tamper- | | | | | |
| | | evident (TEP) | | | | | |
| | | packaging | | | | | |
| | | requirements | | | | | |
| Packaging and | II | Line | 6% | 4 | 0.23 | 4 | 0.23 |
| Labelling System | | clearance, | | | | | |
| | | inspection, and | | | | | |
| | | documentation | | | | | |
| Packaging and | II | Product | 6% | 4 | 0.23 | 4 | 0.23 |
| Labelling System | | segregation and | | | | | |
| | | labelling | | | | | |
| Packaging and | II | Sampling | 6% | 4 | 0.23 | 3 | 0.18 |
| Labelling System | | plan and | | | | | |
| | | acceptance | | | | | |
| | | operations for | | | | | |
| | | packaging and | | | | | |
| | | labeling materials | | | | | |
| Packaging and | II | Specifications | 6% | 4 | 0.23 | 4 | 0.23 |
| Labelling System | | for packaging & | | | | | |
| | | labeling materials | | | | | |
| Packaging and | II | Validation of | 6% | 4 | 0.23 | 4 | 0.23 |
| Labelling System | | packaging & | | | | | |
| | | labeling | | | | | |
| | | operation | | | | | |
| Packaging and | III | Control of | 2% | 4 | 0.09 | 4 | 0.09 |
| Labelling System | | issuance of | | | | | |
| | | labeling, | | | | | |
| | | examination of | | | | | |
| | | issued labels and | | | | | |
| | | reconciliation of | | | | | |
| | | used labels | | | | | |
| Packaging and | III | Controls and | 2% | 4 | 0.09 | 4 | 0.09 |
| Labelling System | | management of | | | | | |
| | | packaging | | | | | |
| | | operations | | | | | |
| Packaging and | III | Monitoring | 2% | 4 | 0.09 | 4 | 0.09 |
| Labelling System | | of printing | | | | | |
| | | devices | | | | | |
| Packaging and | III | Physical/spatial | 2% | 4 | 0.09 | 4 | 0.09 |
| Labelling System | | separation | | | | | |
| | | between different | | | | | |
| | | labeling and | | | | | |
| | | packaging lines | | | | | |
| Packaging and | III | QA In- | 2% | 4 | 0.09 | 4 | 0.09 |
| Labelling System | | process control | | | | | |
| | | checks for | | | | | |
| | | labeling / | | | | | |
| | | packaging | | | | | |
| | | operations | | | | | |
| Packaging and | III | Storage, | 2% | 4 | 0.09 | 4 | 0.09 |
| Labelling System | | issue, inspection, | | | | | |
| | | & reconciliation | | | | | |
| | | of labels and | | | | | |
| | | printed materials, | | | | | |
| | | returns after issue | | | | | |
| Packaging and | III | Validation | 2% | 4 | 0.09 | 4 | 0.09 |
| Labelling System | | and security of | | | | | |
| | | computerized / | | | | | |
| | | automated | | | | | |
| | | labeling/packaging | | | | | |
| | | processes | | | | | |
| Production | I | Component | 6% | 4 | 0.22 | 4 | 0.22 |
| System | | cleaning | | | | | |
| | | validation | | | | | |
| Production | I | Dispensary | 6% | 4 | 0.22 | 4 | 0.22 |
| System | | operations | | | | | |
| Production | I | Equipment | 6% | 3 | 0.17 | 3 | 0.17 |
| System | | cleaning & use | | | | | |
| | | logs | | | | | |
| Production | I | In-process | 6% | 4 | 0.22 | 3 | 0.17 |
| System | | and final product | | | | | |
| | | specifications | | | | | |
| Production | I | Master | 6% | 4 | 0.22 | 4 | 0.22 |
| System | | Manufacturing | | | | | |
| | | Instructions | | | | | |
| | | and Records | | | | | |
| Production | I | Process | 6% | 3 | 0.17 | 4 | 0.22 |
| System | | validation | | | | | |
| Production | I | Process | 6% | 3 | 0.17 | 4 | 0.22 |
| System | | validation | | | | | |
| | | (including CSV | | | | | |
| | | and security of | | | | | |
| | | computerized/ | | | | | |
| | | automated | | | | | |
| | | processes) | | | | | |
| Production | I | Validation of | 6% | 3 | 0.17 | 4 | 0.22 |
| System | | homogeneity | | | | | |
| Production | I | Validation of | 6% | 4 | 0.22 | 4 | 0.22 |
| System | | shelf life (stability | | | | | |
| | | testing) | | | | | |
| Production | II | Adequate | 4% | 4 | 0.16 | 4 | 0.16 |
| System | | procedure and | | | | | |
| | | practice for | | | | | |
| | | charge-in of | | | | | |
| | | components | | | | | |
| Production | II | Development | 4% | 4 | 0.16 | 4 | 0.16 |
| System | | products | | | | | |
| | | manufacture and | | | | | |
| | | controls | | | | | |
| | | (Technology | | | | | |
| | | Transfer) | | | | | |
| Production | II | Environment | 4% | 4 | 0.16 | 4 | 0.16 |
| System | | al monitoring | | | | | |
| Production | II | Facility | 4% | 4 | 0.16 | 4 | 0.16 |
| System | | cleaning | | | | | |
| | | validation | | | | | |
| Production | II | In process | 4% | 4 | 0.16 | 3 | 0.12 |
| System | | controls | | | | | |
| Production | II | Justification | 4% | 4 | 0.16 | 4 | 0.16 |
| System | | and consistency | | | | | |
| | | of in-process | | | | | |
| | | specifications and | | | | | |
| | | drug product final | | | | | |
| | | specifications | | | | | |
| Production | II | Key | 4% | 4 | 0.16 | 3 | 0.12 |
| System | | manufacturing | | | | | |
| | | processes | | | | | |
| Production | II | Pre-process | 4% | 3 | 0.12 | 4 | 0.16 |
| System | | checks, line | | | | | |
| | | clearance, & | | | | | |
| | | equipment | | | | | |
| | | cleaning | | | | | |
| Production | II | Yield | 4% | 4 | 0.16 | 4 | 0.16 |
| System | | calculations and | | | | | |
| | | acceptance limits | | | | | |
| | | at critical process | | | | | |
| | | stages | | | | | |
| Production | III | Control of | 3% | 4 | 0.10 | 4 | 0.10 |
| System | | microbiological | | | | | |
| | | spoilage | | | | | |
| Production | III | Gowning | 3% | 3 | 0.08 | 3 | 0.08 |
| System | | regimes and | | | | | |
| | | requirements | | | | | |
| Production | III | Identity of | 3% | 4 | 0.10 | 4 | 0.10 |
| System | | equipment | | | | | |
| | | contents, phase of | | | | | |
| | | manufacture/status | | | | | |
| Production | III | Personnel | 3% | 4 | 0.10 | 4 | 0.10 |
| System | | entry qualification | | | | | |
| Production | III | Personnel | 3% | 4 | 0.10 | 3 | 0.08 |
| System | | hygiene & | | | | | |
| | | medical fitness | | | | | |
| Production | III | Process | 3% | 4 | 0.10 | 4 | 0.10 |
| System | | descriptions (by | | | | | |
| | | process) | | | | | |
| Quality | I | ADE | 5% | 4 | 0.20 | 4 | 0.20 |
| Management | | Management | | | | | |
| Systems | | | | | | | |
| Quality | I | BMR/BPR | 5% | 4 | 0.20 | 4 | 0.20 |
| Management | | reviews, approval, | | | | | |
| Systems | | archival and | | | | | |
| | | retreival | | | | | |
| Quality | I | Customer | 5% | 3 | 0.15 | 4 | 0.20 |
| Management | | complaint | | | | | |
| Systems | | management | | | | | |
| Quality | I | Management | 5% | 4 | 0.20 | 3 | 0.15 |
| Management | | of CAPA (FAR, | | | | | |
| Systems | | FAR closure and | | | | | |
| | | concomitant | | | | | |
| | | CAPA) | | | | | |
| Quality | I | Non- | 5% | 4 | 0.20 | 4 | 0.20 |
| Management | | conforming | | | | | |
| Systems | | materials, root | | | | | |
| | | cause | | | | | |
| | | investigation and | | | | | |
| | | impact assessment | | | | | |
| Quality | I | Previous | 5% | 4 | 0.20 | 4 | 0.20 |
| Management | | regulatory | | | | | |
| Systems | | obervation | | | | | |
| | | closure, EIR and | | | | | |
| | | Regulatory | | | | | |
| | | compliance | | | | | |
| Quality | I | Recall | 5% | 4 | 0.20 | 4 | 0.20 |
| Management | | Management | | | | | |
| Systems | | | | | | | |
| Quality | I | Risk | 5% | 3 | 0.15 | 4 | 0.20 |
| Management | | assessment and | | | | | |
| Systems | | Mitigation Plans | | | | | |
| Quality | I | Stability | 5% | 4 | 0.20 | 4 | 0.20 |
| Management | | programme | | | | | |
| Systems | | management | | | | | |
| Quality | I | Technology | 5% | 4 | 0.20 | 4 | 0.20 |
| Management | | transfer | | | | | |
| Systems | | | | | | | |
| Quality | II | Change | 12% | 2 | 0.23 | 4 | 0.47 |
| Management | | control (In plant | | | | | |
| Systems | | modification, | | | | | |
| | | material handling, | | | | | |
| | | packaging & | | | | | |
| | | labelling etc.) | | | | | |
| Quality | II | Deviation | 12% | 2 | 0.23 | 2 | 0.23 |
| Management | | Management | | | | | |
| Systems | | (product, process | | | | | |
| | | & utilities) | | | | | |
| Quality | II | Product | 12% | 2 | 0.23 | 4 | 0.47 |
| Management | | disposition | | | | | |
| Systems | | | | | | | |
| Quality | III | Annual | 2% | 3 | 0.06 | 3 | 0.06 |
| Management | | Product Review, | | | | | |
| Systems | | controls charts | | | | | |
| | | and summary | | | | | |
| Quality | III | Documentation | 2% | 2 | 0.04 | 4 | 0.09 |
| Management | | management, | | | | | |
| Systems | | Record | | | | | |
| | | management | | | | | |
| | | & Archive - | | | | | |
| | | check for data | | | | | |
| | | traceability and | | | | | |
| | | real time | | | | | |
| | | recordings, | | | | | |
| | | alignment of e- | | | | | |
| | | copies and | | | | | |
| | | respective hard | | | | | |
| | | copies generated | | | | | |
| Quality | III | Management | 2% | 4 | 0.09 | 4 | 0.09 |
| Management | | Review and | | | | | |
| Systems | | Escalation | | | | | |
| | | procedure | | | | | |
| Quality | III | Quality | 2% | 4 | 0.09 | 3 | 0.06 |
| Management | | audits and | | | | | |
| Systems | | auditing | | | | | |
| Quality | III | Quality | 2% | 4 | 0.09 | 3 | 0.06 |
| Management | | Planning | | | | | |
| Systems | | | | | | | |
| Quality | III | Training & | 2% | 3 | 0.06 | 4 | 0.09 |
| Management | | Qualification | | | | | |
| Systems | | Management | | | | | |
| Quality | III | Validation | 2% | 4 | 0.09 | 4 | 0.09 |
| Management | | management | | | | | |
| Systems | | including VMP | | | | | |
| | | and Q &V QA | | | | | |
| | | peer review | | | | | |
| | | | | System | 19.62 | System | 22.63 |
| | | | | Score | | Score | |

By improving the System Score, addressing specific categories/parameters as reflected in TABLE 8, the PREDICT™ rating for Site #3 was improved from 3 (Yellow zone) to 5 (Dark Green zone).

Similar to Site #3, actions/interventions were adopted for 12 pharmaceutical manufacturing sites.

TABLE 9 depicts the Initial System Score, CALCULUS™ Score and SME Score calculated to arrive at PREDICT™ rating for 12 pharmaceutical manufacturing sites.

TABLE 9

| Site | Site1 | Site 2 | Site 3 | Site 4 | Site 5 | Site 6 | Site 7 | Site 8 | Site 9 | Site 10 | Site 11 | Site 12 |
|---|---|---|---|---|---|---|---|---|---|---|---|---|
| System Score | 10.00 | 21.53 | 19.62 | 20.58 | 23.06 | 15.00 | 20.24 | 19.05 | 20.30 | 18.50 | 19.57 | 17.00 |
| CALCULUS ™ | 65 | 65 | 75 | 78.34 | 77.26 | 65 | 75 | 62.25 | 65.62 | 66.75 | 75 | 42.45 |
| Score | | | | | | | | | | | | |
| SME Score | 10.00 | 8.00 | 7.00 | 7.00 | 8.00 | 7.00 | 8.50 | 7.00 | 7.00 | 8.00 | 8.00 | 8.00 |
| Y value* | 73.71 | 70.94 | 74.75 | 76.42 | 77.07 | 69.75 | 76.53 | 68.38 | 70.06 | 71.81 | 75.94 | 59.66 |

*Y value is determined based on the mathematical formula provided in the earlier aspect of PREDICT ™

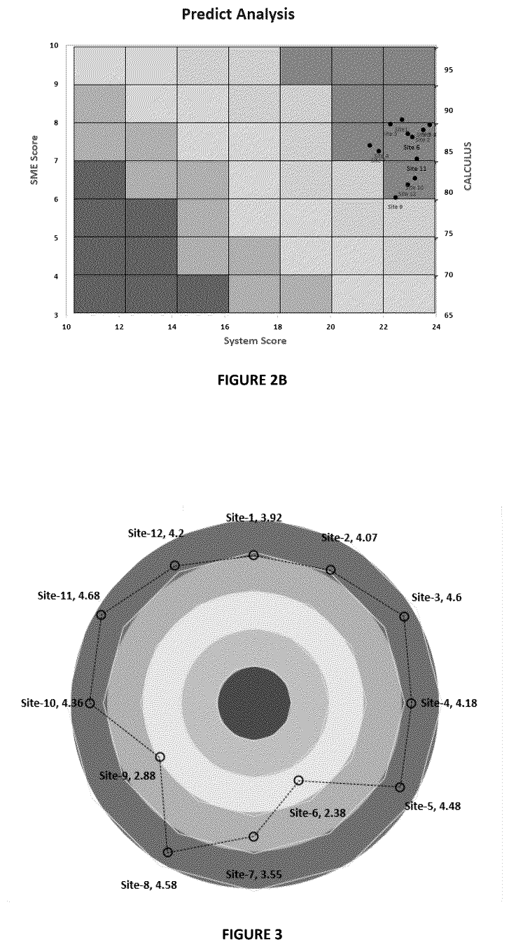

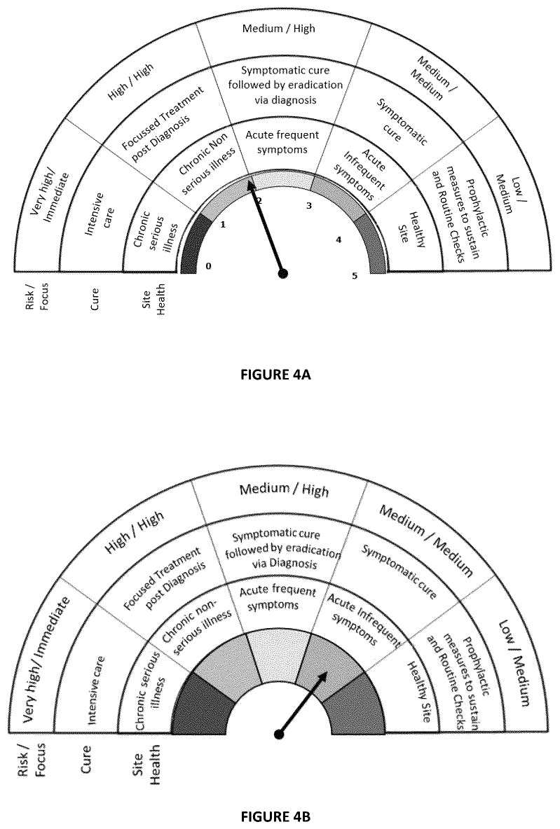

FIG. 2A represents the graphical plotting of the values indicated in the TABLE 9 above.

TABLE 10 depicts the Improved System Score, CALCULUS™ Score and SME Score calculated to arrive at PREDICT™ rating for 12 pharmaceutical manufacturing sites.

TABLE 10

| Site | Site1 | Site 2 | Site 3 | Site 4 | Site 5 | Site 6 | Site 7 | Site 8 | Site 9 | Site 10 | Site 11 | Site 12 |
|---|---|---|---|---|---|---|---|---|---|---|---|---|
| System Score | 22.68 | 23.49 | 22.63 | 21.47 | 22.91 | 23.07 | 21.81 | 23.73 | 22.45 | 23.18 | 23.26 | 22.90 |
| CALCULUS ™ | 84.50 | 86.71 | 92.85 | 87.64 | 85.87 | 80.25 | 86.33 | 87.96 | 75.00 | 75.00 | 75.00 | 78.16 |
| Score | | | | | | | | | | | | |
| SME Score | 9.00 | 8.00 | 7.00 | 7.00 | 8.00 | 9.00 | 7.00 | 8.00 | 7.00 | 8.00 | 9.00 | 7.00 |
| Y value* | 88.89 | 87.64 | 88.35 | 85.75 | 87.22 | 86.77 | 85.09 | 88.27 | 79.43 | 81.79 | 84.14 | 81.01 |

*Y value is determined based on the mathematical formula provided in the earlier aspect of PREDICT ™

FIG. 2B represents the graphical plotting of the values indicated in the TABLE 10 above.

By improvement of System Score, CALCULUS™ Score and/or SME Score, the PREDICT™ rating has been improved.

### Example 3: Determination of SENSOR™ Score

SENSOR™ Score for evaluating quality health of 12 different pharmaceutical manufacturing sites is as indicated below. Each parameter is scored as described in previous aspect of SENSOR™ and listed accordingly in TABLE 11 below:

TABLE 11

| Critical | Parameter | | | | | | | | | | | |
|---|---|---|---|---|---|---|---|---|---|---|---|---|
| Indicator | Site 1 | Site 2 | Site 3 | Site 4 | Site 5 | Site 6 | Site 7 | Site 8 | Site 9 | Site 10 | Site 11 | Site 12 |
| Data | 0.48 | 0.48 | 0.6 | 0.48 | 0.48 | 0.48 | 0.36 | 0.48 | 0.48 | 0.48 | 0.48 | 0.48 |
| Integrity | | | | | | | | | | | | |
| Compliance | | | | | | | | | | | | |
| Product | 0.5 | 0.5 | 0.4 | 0.3 | 0.4 | 0.5 | 0.1 | 0.3 | 0.3 | 0.4 | 0.5 | 0.5 |
| Quality | | | | | | | | | | | | |
| Complaints | | | | | | | | | | | | |
| Invalidated | 0.4 | 0.4 | 0.4 | 0.4 | 0.4 | 0.4 | 0.32 | 0.4 | 0.4 | 0.4 | 0.4 | 0.4 |
| OOS | | | | | | | | | | | | |
| CAPA | 0.36 | 0.48 | 0.6 | 0.6 | 0.6 | 0.24 | 0.36 | 0.6 | 0.12 | 0.6 | 0.6 | 0.48 |
| Closure rate | | | | | | | | | | | | |
| Process OOS | 0.32 | 0.08 | 0.4 | 0.32 | 0.4 | 0.08 | 0.08 | 0.4 | 0.32 | 0.4 | 0.4 | 0.24 |
| or RFT | | | | | | | | | | | | |
| Investigations | 0.21 | 0.28 | 0.35 | 0.35 | 0.35 | 0.07 | 0.35 | 0.35 | 0.35 | 0.35 | 0.35 | 0.35 |
| Closure rate | | | | | | | | | | | | |
| Stability | 0.5 | 0.5 | 0.5 | 0.5 | 0.5 | 0.1 | 0.5 | 0.5 | 0.2 | 0.5 | 0.5 | 0.4 |
| OTIF | | | | | | | | | | | | |
| Change | 0.32 | 0.4 | 0.4 | 0.4 | 0.4 | 0.16 | 0.32 | 0.4 | 0.24 | 0.4 | 0.4 | 0.4 |
| Control | | | | | | | | | | | | |
| Closure rate | | | | | | | | | | | | |
| Deviation | 0.48 | 0.6 | 0.6 | 0.6 | 0.6 | 0.12 | 0.48 | 0.6 | 0.24 | 0.6 | 0.6 | 0.6 |
| Closure rate | | | | | | | | | | | | |
| SOP validity | 0.15 | 0.15 | 0.15 | 0.15 | 0.15 | 0.03 | 0.06 | 0.15 | 0.03 | 0.15 | 0.15 | 0.15 |
| Audit Score | 0.2 | 0.2 | 0.2 | 0.2 | 0.2 | 0.2 | 0.5 | 0.4 | 0.2 | 0.2 | 0.3 | 0.2 |
| Total Score | 3.92 | 4.07 | 4.6 | 4.3 | 4.48 | 2.38 | 3.43 | 4.58 | 2.88 | 4.48 | 4.68 | 4.2 |

FIG. 3 represents comparative evaluation of SENSOR™ score for multiple (12) sites.

Actions and SENSOR™ score improvement for Site #9:

Previous SENSOR™ Score of Site #9 was evaluated as below:

TABLE 12
“SITE #9” - Baseline score

| Overall Score | | | | |
|---|---|---|---|---|
| Overall Site Health | | | | 1.9 |
| Sr. No. | Indicator | Weightage | Rating | Score |
| 1 | Data Integrity Compliance | 12.0% | 2 | 0.24 |
| 2 | Product Quality Complaints | 10.0% | | 0.10 |
| 3 | Invalidated OOS | 8.0% | | 0.40 |
| 4 | CAPA Closure rate | 12.0% | | 0.12 |
| 5 | Process OOS or RFT | 8.0% | | 0.08 |
| 6 | Investigations Closure rate | 7.0% | | 0.35 |
| 7 | Stability OTIF | 10.0% | | 0.10 |
| 8 | Change Control Closure rate | 8.0% | | 0.08 |
| 9 | Deviation Closure rate | 12.0% | 2 | 0.24 |
| 10 | SOP validity | 3.0% | | 0.03 |
| 11 | Audit Score | 10.0% | 2 | 0.20 |

Following actions/interventions were adopted to improve the scores indicated in TABLE 12:
A detailed gap assessment with respect to DI controls was done by third party consultant done followed by focused execution of the identified CAPA thereby improving the DI score.
A specific team was assigned on-site to support the site in performing the analysis of overdue Stability samples.
Support deployed of a remote team to help in tracking and monitoring the open QMS records (complaint, deviation, change control, CAPA) and supporting the site team in review and closure to bring the situation back in control. Currently this was handed over to site and site is handling this independently.
A focused training to the SMEs on audit management helped improving the audit facing and reduced the number of observations during the audits.

### SENSOR™ Score of Site #9 Post Improvement:

TABLE 13
“SITE #9” - Baseline score

| Overall Score | | | | |
|---|---|---|---|---|
| Overall Site Health | | | | 3.55 |
| Sr. No. | Indicator | Weightage | Rating | Score |
| 1 | Data Integrity Compliance | 12.0% | 4 | 0.24 |
| 2 | Product Quality Complaints | 10.0% | 1 | 0.10 |
| 3 | Invalidated OOS | 8.0% | 4 | 0.40 |
| 4 | CAPA Closure rate | 12.0% | 3 | 0.12 |
| 5 | Process OOS or RFT | 8.0% | 1 | 0.08 |
| 6 | Investigations Closure rate | 7.0% | 5 | 0.35 |
| 7 | Stability OTIF | 10.0% | 5 | 0.10 |
| 8 | Change Control Closure rate | 8.0% | 4 | 0.08 |
| 9 | Deviation Closure rate | 12.0% | 4 | 0.24 |
| 10 | SOP validity | 3.0% | 2 | 0.03 |
| 11 | Audit Score | 10.0% | 5 | 0.20 |

The methodology enabled to identify the appropriate parameters requiring improvement and the level meter helped identify the improvement achieved.

What is claimed is:

1. A method for evaluating the data integrity compliance, audit readiness and/or quality health of a pharmaceutical manufacturing site by determining the CALCULUS™ score, PREDICT™ rating and SENSOR™ score respectively.

2. The method for evaluating the data integrity compliance of a pharmaceutical manufacturing site by determining the CALCULUS™ score according to claim 1, wherein the method comprises the steps of:
(i) determining data integrity compliance score (CALCULUS™ score) of a pharmaceutical manufacturing site, which is computed as a sum of a data integrity (DI) score as a first component and a level of automation score as a second component;
wherein; the first component is determined using data integrity compliance checklist based on 163 parameters categorized under:
a) Recording and collection of data
b) Original record/True copy
c) Excluding data,
d) Data Processing
e) Data transfer/migration
f) Data Governance
g) Data Integrity Risk Assessment (DTRA)
h) Computerized System transactions
i) Computerized system User access/System Administrator role
j) Audit Trail
k) Electronic Signatures
l) Data review and approval
m) Data Retention
n) Back up and Archive
o) File structure
p) Validation
q) IT supplier and Service provider
r) Quality Management Systems (QMS)
s) Calibration
t) Quality Control
u) Standalone Systems
v) Trainings
w) Manufacturing process;
wherein the first component or DI score is computed as 85% of the total checklist score;
wherein the second component for measuring the data integrity compliance is related to internal need for increasing and elevating automation to reduce possibility of errors, based on the level of automation at site comprising scoring out of 5% for Quality Control, 5% for Quality Management System and 5% for Manufacturing;
wherein the second component is the sum of the scores for Quality Control, Quality Management System and Manufacturing, out of a total of 15%;
wherein the pharmaceutical manufacturing site is provided a rating of A+, A, B or C based on the DI compliance score (CALCULUS™ score);
wherein,
(i) the rating of A+ is assigned to a site with DI score of >99%;
(ii) the rating of A is assigned to a site with DI score of >85-98.99%;
(iii) the rating of B is assigned to a site with DI score of 70-84.99%;
(iv) the rating of C is assigned to a site with DI score of <70%, and
the qualifying rating for the site as data integrity compliant is A+ or A; with need of improvement for ratings of B or C.

3. The method according to claim 2, wherein the first component or DI score is determined using data integrity compliance checklist of 163 parameters:
(a) Recording and collection of data:
1. Checking whether an Standard Operating Procedure (SOP) or guideline on Good Documentation Practices exists, whether it covers aspects of ‘ALCOA’ (Attributable, Legible, contemporaneous, Original, Accurate) & ‘+’ (Complete, Consistent, Enduring, Available) principle and whether they are followed;
2. Checking whether an SOP for Issuance, document control and reconciliation & destruction of Blank forms/Batch records, paginated logbooks and Lab Notebooks exists;
3. Checking whether a process of ensuring that always the current copy of SOP is in circulation and the obsolete SOPs are withdrawn in timely manner with appropriate reconciliation is in place;
4. Checking whether uncontrolled copies are issued and if issued, whether the SOP defines the procedure on issuance and handling of uncontrolled copies;
5. Checking whether issuance of Batch Manufacturing Record (BMR), Batch Packaging Record (BPR) and lab notebooks is controlled by QA, Document controller or by electronic module and whether each page is either stamped and signed or bears a unique identification with page number (auto generated);
6. Checking whether there is a proper control over replacement of any page(s) and in times of genuine need, whether this is done through a validated system control and whether replacement is recorded adequately;
7. Checking whether there is a logbook maintained for issuance of forms, templates or similar documents;
8. Checking whether all Quality related activities are recorded at the same time when they are performed, and whether BMR, BPR and Lab notebooks are recorded contemporaneously;
9. Checking whether the site has a practice of recording data on pieces of paper that will be discarded after the data are transcribed to a permanent laboratory notebook;
10. Checking whether there is a practice of use of scribes to record activity on behalf of another operator, if such practice exists, whether it is verified and documented contemporaneously or retrospectively with adequate justification, and whether this is defined as part of procedure;
11. Checking whether there exist practices to ensure in routine and confirm that there is no backdating, postdating, omitting negative data (Out of Specification (OOS) or eliminating outliers) or releasing failing product, manipulation, deliberated hidden information or missing signatures on any Good Manufacturing Practices (GMP) or Quality Management System (QMS) record;
12. Checking whether the team has appropriate level of process understanding and technical knowledge of systems used for data collection and recording, including their capabilities, limitations and vulnerabilities;
13. Checking whether the system has feature to not obscure previously recorded information during record change by authorized personnel;
14. Checking whether adequate controls exist in case of run abortion in High Performance Liquid Chromatography (HPLC) systems;
15. Checking whether electronic system (HPLC, QMS application, server) downtime are documented and investigated for the reason;
16. Checking whether key process equipment have controls that measure its operating range of critical process parameters and whether this range is reviewed during batch release decision;
17. Checking whether hybrid systems are used and if used, whether they are clearly documented for which data set is to be reviewed and retained;
18. Checking whether the system has the ability to generate accurate and complete data records in both paper and electronic format (human readable) which could be readily available and accessible (if requested by national competent authorities);
19. Checking whether the system generated report specifies the time zone where this data is used across multiple sites;
20. Checking whether there exists a practice of retention of incomplete or erroneous forms as permanent records along with justification of their replacement and whether this is governed by SOP;
21. Checking whether records or procedures are accessible to locations where activities take place;
(b) Original record or True copy:
1. Checking whether there exists a system or procedure of Handling, Review and Retention of original records-dynamic (electronic) records and static (printed/manual) records generated at Site;
2. Checking whether the data (or a true copy) generated in paper format is retained in the form of scan copies;
3. Checking whether there exists any data at the site where it is not feasible to retain in original form, and if it such data exists, whether the risk assessment is documented and whether appropriate form of data is retained as true copy;
4. Checking whether a true copy of the original data is maintained securely throughout the records retention period, whether there is a risk assessment performed in case of destruction of original record and whether this is governed by SOP;
5. Checking whether there exists a process of risk assessment of data where the data obtained requires manual observation to record the results of manual titration, visual interpretation of environmental monitoring plates and similar results;
6. Checking whether manual transcription for above point are verified by second person or validated system;
7. Checking whether the true copy is stored in different electronic format to the original record and if stored in a different format, whether retention of meta data and audit trail are ensured;
(c) Excluding data:
1. Checking whether there is a process for exclusion of data and if such process exists, whether there is a process to demonstrate through valid scientific justification that the data are not representative of the quantity measured, sampled or acquired;
(d) Data Processing:
1. Checking whether reprocessing of chromatographic data is performed and if reprocessed, whether there exist procedures in place to handle and document such reprocessing;
2. Checking whether a copy is saved only of the final results from reprocessed laboratory chromatography and not the entire sequence;
3. Checking whether manual integration of chromatograms is practiced and whether the software allows saving of manually integrated chromatograms or test results on desktops or temporary locations;
4. Checking whether there exist controls to track or check if trial samples are analysed before the original sample;
(e) Data transfer or migration:
1. Checking whether the site team has a robust and validated data transfer or migration or retention procedure to ensure that data integrity is maintained during the data lifecycle;
2. Checking whether there is a data migration audit log, whether there were challenge tests performed on the migrated data to ensure there is no alteration at each stage of data generation;
3. Checking whether the migration failures (if any) are logged through QMS;
(f) Data Governance:
1. Checking whether there exists an SOP on Data Integrity which defines data governance at the site and whether the data ownership and accountability is defined in the SOP;
2. Checking whether the procedure describes the method to handle data integrity incident observed (if any) at the site;
3. Checking whether periodic audits are conducted for data integrity failures within the organisation's systems;
4. Checking whether the DI behaviour aspects are a part of performance indicators;
5. Checking whether the site performs a data governance review as part of their vendor assurance programme;
(g) Data Integrity Risk Assessment (DIRA):
1. Checking whether DIRA is performed at the site;
2. Checking whether data integrity risk assessment (or equivalent) considers factors required to follow a process or perform a function including not only a computerised system but also the supporting people, guidance, training and quality systems;
3. Checking whether there are procedures in place to minimize the potential risk to data integrity including identifying the residual risk using risk management techniques;
4. Checking whether the site has a core team or SME who performs the DIRA;
5. Checking whether the DIRA report highlights areas for remediation including,
1) documentation of prioritisation of actions (including acceptance of an appropriate level of residual risk),
2) communication to management, subject to review, and
3) implementing risk-reducing short-term measures in situations where long-term remediation actions are identified to provide acceptable data governance in the interim;
(h) Computerized System transactions:
1. Checking whether electronic modules (document management or Trackwise) used for QMS system are 21 CFR part 11/EU annex 11 compliant;
2. Checking whether the systems used at site are capable of saving data to permanent memory before prompting users to make changes;
3. Checking whether in case of transactional systems, any combination of multiple unit operations into a combined single transaction is avoided and whether the time interval before saving the data in computerized system is minimized;
4. Checking whether there exists a process of changing time due to day light savings and if it exists, whether there is a procedure and controls in place, whether this is done by authorized individual and whether this is documented;
5. Checking whether the date and time change setting is disabled at all computer workstations on site;
6. Checking whether time management system is validated to ensure correct logging of date and time of attendance;
7. Checking whether there exists a procedure detailing the password complexity, number of wrong password attempts and password change frequency (system generated);
8. Checking whether password change prompt is activated asking for the change on fixed periodicity;
9. Checking whether the computer system gets timed out within limited time if not worked on as per Information Technology (IT) policy requiring re-entering of password, and if the user leaves the workstation, whether procedures and/or automatic controls define and ensure that it is treated as a non-continuous session;
(i) Computerized system User access/System Administrator role:
1. Checking whether there exists a procedure to create and maintain user account in electronic system and whether the system has control to avoid duplicate user creation;
2. Checking whether there exists an approved user access matrix for all the users and whether this access matrix exactly corresponds with the actual user privileges while using the systems;
3. Checking whether the access controls are applied to both the operating system and application levels and whether the access privileges list are periodically updated;
4. Checking whether sufficient controls are available to prevent unauthorized access to system or to change or delete the data;
5. Checking whether there exist instruments or computer systems that have shared logins, whether generic user or system admin access is used for generating, amending or storing data for Good Practices (GxP) systems and whether there are appropriate procedures available to ensure user controls and data integrity;
6. Checking whether all users have individual login ID and password to the respective instrument or system where electronic data or record is generated or maintained and in absence thereof, whether appropriate controls are in place to ensure data integrity;
7. Checking whether the system administrator for all computerised systems is independent from the user and whether same person acting as user as well as administrator;
8. Checking whether there are multiple system administrators for a single system and if there are such multiple system administrators, whether they are restricted to minimum number of people and whether they use individual ID and password;
9. Checking whether only system administrator has rights to alter files and settings and if they have such rights, whether there exists record of all the actions generated;
10. Checking whether appropriate assessments and controls are available for systems partially used for GxP purposes, approved suppliers, stock status, location and transaction histories and similar purposes;
11. Checking whether if no suitable alternative computerised system is available, whether equivalent controls are provided by third-party software or a paper-based method of providing traceability (with version control);
(j) Audit Trail:
1. Checking whether audit trail is activated for all your computerised systems;
2. Checking whether Audit trail includes the following:
User Name
Date and Time
Reason for change
Integration parameter used in case of chromatograph
Reprocessing details
Change History
Changes to sample run sequence (as applicable)
Change in critical process parameters to achieve a more desirable result (as applicable)
esignature;
3. Checking whether user is able to amend (switch on/off) the audit trail settings and edit the raw data (including chromatographic data) for any computerized systems;
4. Checking whether there exist optimal controls and measures to evaluate data generated by the message centre in the form of an audit trail related to alarms, warning, error or run time data and whether there is an SOP governing the same;
5. Checking whether the electronic record has system generated date and time stamp, and if the time on the system and report is synchronized;
6. Checking whether there exists a procedure to periodically review the appropriate audit trails and data integrity review to verify adherence to written procedures (to verify privileges assigned to users, creation of project folders, data generated and maintained correctly);
7. Checking whether there exists a procedure in place that describes audit trail review requirements:
frequency and documentation of its review,
date of review, reviewed by and outcome of review, identification of actions required in case of non compliance,
indicators of unauthorized changes;
8. Checking whether there are enough controls to ensure that all original chromatographic data is restricted from editing once finalized or approved or locked;
9. Checking whether the editing of point (j)8 is detectable in audit trails and whether such aspects are considered as part of review of audit trail;
(k) Electronic Signatures:
1. Checking whether there exists an SOP or policy on use or handling of e-signatures;
2. Checking whether there exists an appropriate validation of signature process associated with the system;
3. Checking whether a record is maintained of all the associates who apply their e-signature instead of their handwritten signatures;
4. Checking whether all the computerised systems where electronic signatures are applied, document the specific person or title who signed the records electronically along with date and time stamp and the meaning of the signature (verified or approved);
5. Checking whether there exists a process of inserting image of the signature or a footnote indicating that the document has been electronically signed (where this has been entered by a means other than the validated electronic signature process);
(l) Data review and approval:
1. Checking whether there exists a procedure that describes the process for review and approval of data and whether the raw data is accessible with appropriate control to person performing data checking activities on system;
2. Checking whether the data review includes risk based review of relevant meta data including audit trails and whether the same is documented;
3. Checking whether there exists a practice of thorough data review for all applicable electronic source data to verify scientific integrity of the reported results, to verify adherence to procedures or test methods and to verify that all results are reported and accounted for; whether there is an SOP governing the same;
4. Checking whether the equipment raw data is periodically reviewed, reconciled against paper records and extracted as electronic data where the equipment does not store electronic data permanently and only holds a certain volume before overwriting;
5. Checking whether there is any procedure for periodic review or audit of electronic data generated, which verifies the effectiveness of existing control measures and the possibility of unauthorised activity;
6. Checking whether there are controls to verify the reported data and actual data;
7. Checking whether customized reports in computer systems are validated and locked to prevent changes;
(m) Data Retention:
1. Checking whether there is a retention policy for of all records (manual & electronic), soft data and meta data, and whether it covers periodicity for back up and retention of both electronic and paper records along with meta data;
2. Checking whether there are procedures for destruction of data (blank forms, reports, training records, work sheets, raw data files, logbooks) which consider data criticality;
3. Checking whether there are data and document retention arrangements available to ensure the protection of records from deliberate or inadvertent alteration or loss;
4. Checking whether the data retention process of the sites include:
verification and availability of copies of all raw data (lab note book, logs, balance printouts), metadata,
check for availability of relevant audit trail and result files,
any variable software or system configuration settings specific to each record, and all data processing runs (including methods and audit trails) necessary for reconstruction of a given raw data set;
5. Checking whether metadata includes following components while retention:
a date or time stamp for when the data were acquired,
a user ID of the person who conducted the test or analysis that generated the data,
instrument ID used to acquire the data,
audit trails;
6. Checking whether BMRs and BPRs are centrally archived under Quality Assurance (QA) custody. whether access to this is controlled and restricted (manually controlled or electronically controlled through combination lock, biometrics or access card);
7. Checking whether there is any data generation through photograph, image or any other media and if there is such data generation, whether there are procedures and controls to maintain their storage throughout the lifecycle;
8. Checking whether in case, where the original format cannot be retained due to degradation issues, whether there are alternative mechanisms for recording (photography or digitisation) and subsequent storage considered and selection rationale documented;
9. Checking whether the data (or a true copy) generated in paper format is retained by using a validated scanning process, and if so retained, whether there is a documented process in place to ensure that the outcome is a true copy;
10. Checking whether there are adequate controls to prevent data tamper or deletion and data loss within the software or outside the software application;
11. Checking whether the soft data can be located, retrieved, presented and interpreted in its original or acceptable form;
(n) Back up and Archive:
1. Checking whether validated procedures are available for data backup and archive, and if available, whether it includes,
i. periodic integrity check,
ii. accuracy of backup data,
iii. readability of data or metadata and
iv. the ability to restore the data as per its original validated state including legacy systems to confirm the continued support of legacy computerised systems;
2. Checking whether a procedure is in place for archival of records prior to decommissioning the computerised system;
3. Checking whether data security controls exist on manufacturing software or Programmable Logic Controllers (PLCs), and whether it is backed up periodically to prevent data loss in case of failure;
4. Checking where hybrid records are stored and whether references between physical and electronic records are maintained such that full verification of events is possible throughout the retention period;
5. Checking whether the archival area is suitably controlled and whether it is inspected at regular intervals;
6. Checking whether the site is using any third party service provider for archival of data and if used, whether there is a contract in place which defines the responsibilities for archiving and continued readability of the data throughout the retention period;
7. Checking whether the backup strategies for the data owners are documented;
8. Checking whether adequate back up control, disaster management and server redundancy for chromatographic data exists;
9. Checking whether periodic review or retrieval of chromatographic records is performed, if performed whether it is performed with limited access control and whether this is documented;
10. Checking whether disaster management exists for archival area where BMR or BPR are archived;
(o) File structure:
1. Checking whether there exists a process for file structure and naming conventions within the GxP environment for management of electronic data to facilitate effective data review and ensure that all results are reported and accounted for; including naming of folders or files, sequences and samples as applicable;
2. Checking whether there exists a written procedure to control data acquisition in appropriate project folders and whether there exist appropriate access controls on these folders;
(p) Validation:
1. Checking whether computerized systems are validated as per regulatory requirements for their intended purpose;
2. Checking whether the extent of validation is based on justified and documented risk assessment;
3. Checking whether all PLCs containing recipes or work instructions are validated as per Good Automated Manufacturing Practice (GAMP) 5 or for 21 CFR part 11 controls;
4. Checking whether excel work sheets if used in Quality Control (QC) or any other function are validated and whether these cells are locked, access is controlled and saving or copying of these are disabled;
5. Checking whether validation for intended purpose ensures that the steps for generating the custom report accurately reflect those described in the data checking SOP;
6. Checking whether each workflow on all the computer systems or softwares in use are validated;
7. Checking whether the critical steps are defined based on the functionality of system at User Requirement Specification stage of a software;
8. Checking whether computer systems designed to ensure that the execution of critical steps are recorded contemporaneously;
9. Checking whether the functional verification demonstrates that the required information is consistently and completely presented;
10. Checking whether periodic review of validation status of systems is performed and documented;
11. Checking whether there exists a process of retirement of computerized systems governed by an SOP;
(q) IT supplier and Service provider:
1. Checking whether there exists an inventory of all applicable computerised systems both live and retired and whether they are periodically reviewed;
2. Checking whether the site uses any ‘cloud’ or ‘virtual’ services from third party, and if used, whether there is an understanding of the services provided, ownership, retrieval, retention and security of the data;
3. Checking whether the contract or technical agreements of third party vendor include responsibilities of contract giver and acceptor, clause on data ownership, governance and accessibility;
4. Checking whether in case of cloud services, the geographical location laws are considered where the data is physically located;
5. Checking whether there exists a process of auditing the cloud and software service provider;
6. Checking whether there is an external vendor used for generating summary reports for the site or organization, and if such vendor exists whether the vendor is evaluated for the data integrity controls and processes prior to using the information;
7. Checking whether consultants are appointed at the site, and if appointed, whether there exist procedural controls to prevent access to company data through secured network;
8. Checking whether there exists business continuity for all the critical softwares used at site;
9. Checking whether the business continuity arrangement (manual or alternative system) is documented in contract and tested, and whether the audit of the service provider is performed;
(r) Quality Management Systems (QMS):
1. Checking whether changes to qualified electronic system are routed through change management and risk assessment;
2. Checking whether all deviations or incidences immediately upon their discovery, are documented;
3. Checking whether the deviation investigations (not only failures and data errors) include determining and correcting the root cause of the deviation and a thorough impact assessment;
4. Checking whether the Information Technology related incident or deviations within QMS are logged and tracked;
5. Checking whether the OOS handling procedure mentions when retest is permitted and when approval to do so is required;
6. Checking whether there exists a practice of frequent invalidation of OOS, in case of invalidated OOS, whether a thorough investigation is undertaken for all, and whether there are repeated errors for invalidating the OOS;
7. Checking whether there exists an SOP for repeat testing which includes scientific justification and documentation requirements for the same;
8. Checking whether there exists a procedure in place to handle, if any abnormality or incident related to software is observed during operation;
(s) Calibration:
1. Checking whether the simple electronic systems (pH meters, balances, thermometers and similar electronic systems) are calibrated;
(t) Quality Control:
1. Checking whether the site has a practice of trial injection in chromatographic analysis and deletion of sequences;
2. Checking whether there exists a practice of using actual test samples for system suitability or whether any test or equilibration runs are carried out;
(u) Standalone Systems:
1. Checking whether all QC chromatographic systems or standalone instruments (balances, moisture analyser, ultra violet (UV) spectrophotometer, pH meter) are 21 CFR Part 11/EU Annex 11 compliant, and whether there exist entry level controls in these computers followed by system level access controls;
2. Checking whether there exist any standalone instruments (balances, moisture meter, UV spectrophotometer, pH meter) which are not attached with printers, and if they exist, whether they are validated and with adequate controls for capturing, documenting and second level review of the data and audit trails;
3. Checking whether in case of non availability of audit trail functionality (standalone instruments) and individual user account expectations, whether there exists an alternate robust control procedure for data review and retention along with justification, including legacy systems;
4. Checking whether there exist plans to upgrade standalone systems, and whether the required budget is proposed;
5. Checking whether computers are connected to chromatographic system and/or Supervisory Control and Data Acquisition (SCADA) are disabled for download of any other application (excel, power point, word), whether it is used exclusively for the data acquisition system, and whether it is confirmed that it does not allow download or saving of any data and subsequent changes to it;
6. Checking whether there exists a back-up of standalone instruments and whether the mechanism is validated;
(v) Trainings:
1. Checking whether all personnel accessing the system or involved with the system have adequate training and whether their training records are available, whether their responsibilities are defined and documented to carry out their assigned duties;
2. Checking whether Data Integrity training is performed as part of induction for new joinees;
3. Checking whether periodic trainings to all GxP relevant support functions on importance of Data Integrity principle is provided as part of a routine annual training program;
4. Checking whether the personnel who review and conduct data integrity checks are trained and qualified on data integrity requirements;
5. Checking whether the senior management team is trained on data integrity requirements;
6. Checking whether there exists an SOP, policies, trainings or awareness sessions at site that helps prevent sharing of passwords or IDs;
(w) Manufacturing:
1. Checking whether there exist PLC controls or automation to all key manufacturing equipment, such that defective product can be rejected based on defined tolerances;
2. Checking whether the processes and their validation are robust enough and challenged adequately to not produce a non-compliant product;
3. Checking whether there exists a qualified SCADA (where available) system and whether SCADA print is attached in BMR to prevent manual recording;
4. Checking whether there exists an SOP to assess and document PLC alarms based on its criticality (nature, recording the duration and time) from an operational standpoint, and whether this is also reviewed by the quality unit;
5. Checking whether the PLC controls, alarms the user not only when the product is out of specification but when process steps out of the validated range;
6. Checking whether there exist digital clocks to record timing in the raw data or batch record, and if they exist, whether they are centrally controlled or synchronized for recording timed events specifying the time zone;
7. Checking whether for Active Pharmaceutical Ingredient (API) sites, whether critical parameters (temperature, pressure and pH) of reactors are automated, whether it prevents manual recording and whether print out from PLCs for these parameters are attached in BMR;
8. Checking whether equipment cleaning, sanitization and maintenance records are available and traceable at site;
9. Checking whether procedures are in place to monitor the output of the manufacturing processes that may be responsible for causing variability in the characteristics of in-process material and the drug product;
10. Checking whether the documents generated or printed from electronic systems in shopfloor are compliant to 21 CFR part 11; and
wherein, the first component or data integrity compliance checklist score is computed as 85% of the total score of the 163 parameters.

4. The method of evaluating audit readiness of a pharmaceutical manufacturing site by determining the PREDICT™ rating according to claim 1, wherein the method comprises the steps of:
(i) determining first element or system score being assessment of Good Manufacturing Practice (GMP) system compliance via check points based on six systems comprising 132 parameters categorized into three levels namely Patient Risk (Level-I), Product Quality Risk (Level-II) and Compliance Risk (Level-III), wherein the six systems are:
1. Facility And Equipment System,
2. Laboratory Control System,
3. Material System,
4. Packaging and Labelling System,
5. Production System, and
6. Quality Management Systems;
(ii) determining the second element being assessment of subject matter expert (SME) by corporate auditors, which integrates aspects of subject matter expert readiness at pharmaceutical manufacturing sites, and
(iii) determining the third element being data integrity compliance score, CALCULUS™ score of a pharmaceutical manufacturing site;
wherein the predicted outcome is determined by compilation of system score, SME score and CALCULUS™ score of respective site graphically and the interpretation of predicted outcome and score is interpreted to provide the PREDICT™ rating;
wherein,
(a) if the predicted outcome is red (poor), predict rating is 1, then the severity of observation is high and double-digit warning letters could be expected from regulatory body during audit,
(b) if the predicted outcome is amber (moderately poor), predict rating is 2, then the severity of observation is moderately high and 7-10 warning letters could be expected from regulatory body during audit,
(c) if the predicted outcome is yellow (moderate), predict rating is 3, then the severity of observation is moderate and <7 warning letters could be expected from regulatory body during audit,
(d) if the predicted outcome is light green (good), predict rating is 4, then the severity of observation is low and <3 warning letters could be expected from regulatory body during audit, and
(e) if the predicted outcome is dark green (excellent), predict rating is 5, then the severity of observation is indicative of continuous improvement or non-systemic gaps and 0-2 warning letters could be expected from regulatory body during audit.

5. The method according to claim 4, wherein the Facility and Equipment System comprises of the parameters selected from the group consisting of:
1. Equipment cleaning and sanitization,
2. Prevention of cross-contamination, isolation and containment,
3. Air handling systems,
4. Appropriate use of equipment operations substances,
5. Calibration program,
6. Clean rooms control, maintenance and cleaning,
7. Control system for implementing changes in the equipment,
8. Documented investigation into any unexpected discrepancy,
9. Environmental zoning,
10. Equipment surfaces should not be reactive, additive, or absorptive,
11. Facilities Maintenance,
12. Installation Qualification/Operational Qualification/Performance Qualification (IQ/OQ/PQ) of facilities, utilities and equipment,
13. Informational Technology Good Manufacturing Practices (IT GMP), validation and security,
14. Planned preventive maintenance program,
15. Adequacy of equipment design, size, and location,
16. Design guidelines,
17. Equipment identification practices,
18. Facilities Cleaning,
19. Layout & drawings,
20. Lighting, potable water, washing and toilet facilities, sewage and refuse disposal,
21. Pest control,
22. Sanitation of the building,
23. Technical/Design files, and
24. Zone Ownership.

6. The method according to claim 4, wherein the Laboratory Control System comprises of the parameters selected from the group consisting of:
1. Laboratory methods, standards and controls,
2. Qualification and Validation of all Quality Control (QC) methods,
3. Qualification of all Laboratory Equipment,
4. Raw data definition, standards, control and verification,
5. Reference standards management,
6. Retained samples, requirements and management,
7. Specifications, standards, and sampling plans,
8. Stability testing,
9. Acceptance Activities and in-process release,
10. Adequacy of equipment and facility for intended use,
11. Out of specification (OOS) & Out of trend (OOT) Investigations,
12. Sample handling, storage, and integrity,
13. Test data review and authorization,
14. Validation and security of computerized or automated processes,
15. Adequacy of staffing for laboratory operations,
16. Calibration program,
17. Equipment List,
18. Maintenance program,
19. Method validation policy and standards, and
20. Trending, reporting, and statistical quality control.

7. The method according to claim 4, wherein the Material System comprises of the parameters selected from the group consisting of:
1. Bill of Materials,
2. Environmentally controlled storage conditions,
3. Finished product distribution records by lot,
4. Identification & Quality Status,
5. Lot traceability of components making up a batch,
6. Purified Water system control,
7. Specifications and acceptance testing of raw materials,
8. API, excipient, reagent and reference material control,
9. Control of quarantine goods,
10. Control of reject goods,
11. Expiry dating and retest requirements,
12. Incoming goods acceptance checks,
13. Lot numbering control,
14. Pack Range Control—Artwork, components, pack codes,
15. Qualification of cold chain or supply chain,
16. Reconciliation,
17. Reprocessing/rework control,
18. Sampling plan,
19. Supplier management,
20. Authority to Destroy and destruction of records,
21. Control of Distributors, records, Technical Agreements,
22. Control of returned or salvaged goods,
23. Inventory Management,
24. Raw materials segregation and labeling,
25. Testing or validation of supplier's test results for components, containers and closures, and
26. Warehouse controls and First-in-first-out (FIFO).

8. The method according to claim 4, wherein the Packaging and Labelling System comprises of the parameters selected from the group consisting of.
1. Adequate inspection (proofing) of incoming labeling,
2. Control of bulk and unlabelled product,
3. Examination of the labeled finished product,
4. In-process inspection of product,
5. Master Packaging Instructions and Records,
6. Quality Assurance (QA) In-process control checks for labeling or packaging operations,
7. Specifications for packaging & labeling materials,
8. Conformance to tamper-evident packaging (TEP) requirements,
9. Controls and management of packaging operations,
10. Line clearance, inspection, and documentation,
11. Product segregation and labeling,
12. Sampling plan and acceptance operations for packaging and labeling materials,
13. Storage, issue, inspection and reconciliation of labels and printed materials, returns after issue,
14. Validation and security of computerized/automated labelling/packaging processes,
15. Validation of packaging and labeling operation,
16. Control of issuance of labeling, examination of issued labels and reconciliation of used labels,
17. Monitoring of printing devices, and
18. Physical/spatial separation between different labeling and packaging lines.

9. The method according to claim 4, wherein the Production System comprises of the parameters selected from the group consisting of:
1. Component cleaning validation,
2. Dispensary operations,
3. Equipment cleaning & use logs,
4. In process controls,
5. In-process and final product specifications,
6. Justification and consistency of in-process specifications and drug product final specifications,
7. Key manufacturing processes,
8. Master Manufacturing Instructions and Records,
9. Process validation,
10. Process validation (including computer system validation (CSV) and security of computerized/automated processes),
11. Validation of homogeneity,
12. Validation of shelf life (stability testing),
13. Adequate procedure and practice for charge-in of components,
14. Development products manufacture and controls (Technology Transfer),
15. Environmental monitoring,
16. Gowning regimes and requirements,
17. Personnel entry qualification,
18. Personnel hygiene & medical fitness,
19. Pre-process checks, line clearance, and equipment cleaning,
20. Yield calculations and acceptance limits at critical process stages,
21. Control of microbiological spoilage,
22. Facility cleaning validation,
23. Identity of equipment contents, phase of manufacture/status, and
24. Process descriptions (by process).

10. The method according to claim 4, wherein the Quality Management Systems comprises of the parameters selected from the group consisting of:
1. Adverse Drug Event (ADE) Management,
2. Annual Product Review, controls charts and summary,
3. Batch manufacturing record/Batch packing record (BMR/BPR) reviews, approval, archival and retrieval,
4. Customer complaint management,
5. Management of Corrective and preventive action (CAPA), Field Alert Report (FAR), FAR closure and concomitant CAPA,
6. Non-conforming materials, root cause investigation and impact assessment,
7. Previous regulatory observation closure, Establishment Inspection Report (EIR) and Regulatory compliance,
8. Recall Management,
9. Risk assessment and Mitigation Plans,
10. Stability programme management,
11. Technology transfer,
12. Validation management including Validation master plan (VMP) and Quality peer review,
13. Change control (In plant modification, material handling, packaging & labelling etc.),
14. Deviation Management (product, process & utilities),
15. Product disposition,
16. Quality audits and auditing,
17. Documentation management, Record management and Archive—check for data traceability and real time recordings, alignment of e-copies and respective hard copies generated,
18. Management Review and Escalation procedure,
19. Quality Planning, and
20. Training & Qualification Management.

11. The method according to claim 4, wherein the first element or system score is assessed according to the steps comprising of:
(i) associating each of the 132 parameters with a weightage (%) based on its relevance to patient (highest weightage), product and compliance;
(ii) providing a rating to each of the 132 parameters on a scale of 1 to 4 based on level of compliance, wherein:
(a) 1 signifies “Non-compliant process”,
(b) 2 signifies “Partially compliant process”,
(c) 3 signifies “Compliant with improvements”, and
(d) 4 signifies “Highly compliant process”.
(iii) obtaining the system score for each of the parameters as a product of weightage and the rating; and
(iv) obtaining the final system score on a scale of 1 to 24 as an average of the individual scores wherein:
a) Scores above 20 indicates that the site is highly compliant.
b) Scores ranging from 17-20 indicates that the site is compliant with scope of improvement;
c) Scores ranging from 14-16 indicates that the site is partially compliant with gaps; and
d) Scores less than 14 indicate that the site is non complaint.

12. The method according to claim 4, wherein the second element or the SME readiness is provided a score selected from:
(a) “0” Need major change in SME at all levels including quality
(b) “1” Few critical SMEs (audit facing) in quality requires replacement.
(c) “2” Few critical SME (audit facing) within other 5 systems require replacement
(d) “3” Challenge only with 1 or 2 SME (audit facing) which can be bridged by other competent staff under or at Peer level.
(e) “4” Gap in Quality SME (audit facing) but can be trained.
(f) “5” Gap in few other SMEs (audit facing) but can be trained.
(g) “6” SME (mainly Quality) require extensive level of audit facing training while possessing requisite knowledge.
(h) “7” SME (mainly but not only quality) require major level of strategic audit facing training.
(i) “8” SME (mainly but not only quality) require low level of audit facing training.
(j) “9” SME (mainly quality) requires only incidence related direction or guidance during audit to defend our case as required by regulation during inspection when required.
(k) “10” SME (mainly quality) is self-sufficient in changing the course of inspection in our favour due to strong regulatory knowledge and negotiation capability.

13. The method of measuring the quality health of a pharmaceutical manufacturing site according to claim 1, wherein the quality is visually measured on a level meter, SENSOR™, a dynamic site score on a scale from 0 to 5, comprising the steps of:
(a) determining eleven critical quality indicators:
i. Audit Score (sum of System score as determined in step (i) of claim 4 and SME Score as determined in step (ii) of claim 4),
ii. Data integrity compliance score (CALCULUS™ Score) as determined in claim 2,
iii. Product Quality Complaints,
iv. Invalidated Out of Specification (OOS),
v. Corrective Action and Preventive Action (CAPA) closure rate,
vi. Process OOS or Right First Time (RFT),
vii. Investigation closure rate,
viii. Stability On Time in Full (OTIF),
ix. Change Control closure rate,
x. Deviation Closure rate, and
xi. Standard Operating Procedure Validity
(b) assigning weightages to each of the eleven indicators based on their criticality to compliance, product quality, and patient safety.
(c) rating each indicator on a predefined scale and computing the weighted sum to obtain the SENSOR™ score.
(d) interpreting the SENSOR™ score within a five-zone decision matrix, each zone corresponding to predefined levels of site health, audit risk, and required focus of intervention;
wherein, the level meter is demarcated into 5 colored regions corresponding to the score, selected from:
1) Score of 0-1 corresponding to red region indicates that the Site Health has Chronic Serious Illness, Cure required is Intensive Care, Risk is Very High and Focus required is Immediate,
2) Score of 1-2 corresponding to orange region indicating that the Site Health has Chronic Non-serious Illness, Cure required is Focused Treatment post Diagnosis, Risk is High and Focus required is High,
3) Score of 2-3 corresponding to yellow region indicating that the Site Health has Acute Frequent Symptoms, Cure required is Symptomatic cure followed by eradication via Diagnosis, Risk is Medium and Focus required is High,
4) Score of 3-4 corresponding to light green region indicating that the Site Health has Acute In-frequent Symptoms, Cure required is Symptomatic cure, Risk is Medium and Focus required is Medium, and
5) Score of 4-5 corresponding to green region indicating that the Site is Healthy, Cure required is Prophylactic measures to Sustain and Routine Checks, Risk is Low and Focus required is Medium.
